SEQUENCE LISTING (230 sequences)

SEQ ID NO: 1 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 2 - protein, 651 aa, Synthetic construct; identical to SEQ ID NO: 1

SEQ ID NO: 3 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTNNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 4 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKNGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 5 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPTYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 6 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 7 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFSDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 8 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFINKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 9 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 10 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNLRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 11 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 12 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFLQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 13 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFLVKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 14 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNLRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 15 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNFRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 16 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 17 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFINKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 18 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNELINQSLAFINQSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 19 - protein, 651 aa, Synthetic construct
MELLILKANAITTILTAVTFCFASGQNITEEFYQSTCSAVSKGYLSALRTGWYTSVITIELSNIKENKVNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSVSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFINKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 20 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNELINQSLAFINQSDELLSGSGSESQVRQQFSNDIENLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 21 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGNSLYVNGEPIINFYDPLVFPSDEFDASISQVNELINQSLAFINQSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 22 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGNSLYVNGEPIINFYDPLVFPSDEFDASISQVNELINQSLAFINQSDELLSGSGSESQVRQQFSNDIENLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 23 - protein, 651 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNELINQSLAFINQSDELLSGSGSESQVQQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 24 - protein, 530 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGLEVLFQGPHHHHHHHHSAWSHPQFEK

SEQ ID NO: 25 - protein, 689 aa, Synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGGSSGSSGGSDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 26 - protein, 574 aa, Orthopneumovirus hominis
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 27 - protein, 297 aa, Orthopneumovirus hominis
MSKNKDQRTAKTLERTWDTLNHLLFISSCLYKLNLKSVAQITLSILAMIISTSLIIVAIIFIASANHKITSTTTIIQDATNQIKNTTPTYLTQNPQLGISPSNPSDITSLITTILDSTTPGVKSTLQSTTVGTKNTTTTQAQPNKPTTKQRQNKPPSKPNNDFHFEVFNFVPCSICSNNPTCWAICKRIPNKKPGKRTTTKPTKKPTPKTTKKGPKPQTTKSKEAPTTKPTEEPTINTTKTNIITTLLTSNTTRNPELTSQMETFHSTSSEGNPSPSQVSITSEYPSQPSSPPNTPR

SEQ ID NO: 28 - protein, 233 aa, Orthopneumovirus hominis
NHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDHKPQTTKPKEVPTTKPTEEPTINTTKTNIITTLLTNNTTGNPKLTSQMETFHSTSSEGNLSPSQVSTTSEHPSQPSSPPNTTRQ

SEQ ID NO: 29 - protein, 56 aa, Synthetic construct
SGGSSGSSEEEGGSRQNKPNNDFHFEVFNFVPCSICSNNPTCWAICKRIPNKKEEE

SEQ ID NO: 30 - DNA, 22 bp, Synthetic construct
  source 1..22: mol_type = other DNA
tgactgtgaacgttcgagatga

SEQ ID NO: 31 - protein, 7 aa, Synthetic construct
GSGNVGL

SEQ ID NO: 32 - protein, 43 aa, Synthetic construct
RQNKPPNKPNNDFHFEVFNFVPCSICSNNPTCWAICKRIPNKK

SEQ ID NO: 33 - protein, 42 aa, Synthetic construct
RKNPPKKPKDDYHFEVFNFVPCSICGNNQLCKSICKTIPNKK

SEQ ID NO: 34-200 - skipped (167 entries)

SEQ ID NO: 201 - protein, 173 aa, Synthetic construct
ESQVRQQFSKDIEKLLNEQVNKEMQSSNLYMCMSSWSYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 202 - protein, 173 aa, Synthetic construct
ESQVRQQFSKDIEKLLNEQVNKEMQSSNLYMSMSSWSYTHSLDGAGLFLFDHAAEEYEHAKKLIIFLNENNVPVQLTCISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 203 - protein, 173 aa, Synthetic construct
ESQVRQQFSKDIEKLLNEQVNKEMQSSNLYMSMSSWSYTHSLDGAGLFLFDHAAEEYEHAKKLIIFLNENNVPVQLTSISCPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 204 - protein, 173 aa, Synthetic construct
ESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 205 - protein, 173 aa, Synthetic construct
ESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISECINNIVDHAIKSKDHATFNFLQWYVAEQHEEEVLFKDILDKIELIGNENHGLYLADQYVKGIAKSRKS

SEQ ID NO: 206 - protein, 173 aa, Synthetic construct
ESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 207 - protein, 173 aa, Synthetic construct
ESQVRQQFSKDIEKLLNCQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 208 - protein, 173 aa, Synthetic construct
ESQVRQQFSKDIEKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 209 - protein, 166 aa, Helicobacter pylori
LSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 210 - DNA, 22 bp, Synthetic construct
  source 1..22: mol_type = other DNA
tgactgtgaacgttcgagatga

SEQ ID NO: 211 - protein, 191 aa, Trichoplusia ni
TQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 212 - protein, 212 aa, Trichoplusia ni
ADTCYNDVALDCGITSNSLALPRCNAVYGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 213 - protein, 174 aa, Pyrococcus furiosus
MLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 214 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVQQSLLELHKLATDKNDPHLCDFIETHYLNEQVKAIKELGDHVTNLRKMGAPESGLAEYLFDKHTLGDSDQES

SEQ ID NO: 215 - protein, 204 aa, Homo sapiens
MDSKGSSQKGSRLLLLLVVSNLLLPQGVLASSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 216 - protein, 154 aa, Aquifex aeolicus
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 217 - protein, 8 aa, unidentified
  source 1..8: note = Bullfrog linker sequence
ESQVRQQF

SEQ ID NO: 218 - protein, 15 aa, Synthetic construct
CLVPRGSLEHHHHHH

SEQ ID NO: 219 - protein, 156 aa, Escherichia coli
MNIIEANVATPDARVAITIARFNNFINDSLLEGAIDALKRIGQVKDENITVVWVPGAYELPLAAGALAKTGKYDAVIALGTVIRGGTAHFEYVAGGASNGLAHVAQDSEIPVAFGVLTTESIEQAIERAGTKAGNKGAEAALTALEMINVLKAIKA

SEQ ID NO: 220 - protein, 16 aa, Synthetic construct
GGGGSGGGGSGGGGSG

SEQ ID NO: 221 - protein, 28 aa, Synthetic construct
GGSGSGSNSSASSGASSGGASGGSGGSG

SEQ ID NO: 222 - protein, 46 aa, Synthetic construct
GGSGSASSGASASGSSNGSGSGSGSNSSASSGASSGGASGGSGGSG

SEQ ID NO: 223 - protein, 26 aa, Synthetic construct
GGSGSASAEAAAKEAAAKAGGSGGSG

SEQ ID NO: 224 - protein, 32 aa, Synthetic construct
GGSGSASAEAAAKEAAAKEAAAKASGGSGGSG

SEQ ID NO: 225 - protein, 47 aa, Synthetic construct
SGGGSGSASSGASASGSSCSGSGSGSSSASSGASSGGASGGGSGGSG

SEQ ID NO: 226 - protein, 4 aa, Synthetic construct
GGGS

SEQ ID NO: 227 - protein, 8 aa, Synthetic construct
GGGSGGGS

SEQ ID NO: 228 - protein, 20 aa, Synthetic construct
GGGSGGGSGGGSGGGSGGGS

SEQ ID NO: 229 - protein, 5 aa, Synthetic construct
SGSGS

SEQ ID NO: 230 - protein, 6 aa, Synthetic construct
HHHHHH

(19) United States
(12) Patent Application Publication
(10) Pub. No.: US 2025/0059240 A1
(43) Pub. Date: Feb. 20, 2025

(54) Antigenic Respiratory Syncytial Virus Polypeptides

(71) Applicant: Sanofi, Paris (FR)

(72) Inventors: Gary J. Nabel, Cambridge, MA (US); Chih-Jen Wei, Cambridge, MA (US); Kurt Swanson, Cambridge, MA (US); Pradeep Dhal, Cambridge, MA (US); Ram Dharanipragada, Cambridge, MA (US); Magnus Besev, Cambridge, MA (US)

(73) Assignee: Sanofi, Paris (FR)

(21) Appl. No.: 18/775,573

(22) Filed: Jul. 17, 2024

Related U.S. Application Data
(62) Division of application No. 17/061,065, filed on Oct. 1, 2020, now Pat. No. 12,071,454.
Application No. 17/061,065 is a continuation of application No. PCT/US2019/025387, filed on Apr. 2, 2019.
(60) Provisional application No. 62/652,199, filed on Apr. 3, 2018.

(51) Int. Cl. C07K 14/195

(52) U.S. Cl. CPC C07K 14/195; C07K 2319/30

(57) ABSTRACT

This disclosure relates to antigenic respiratory syncytial virus (RSV) polypeptides for use in eliciting antibodies against RSV. Also disclosed are antigenic polypeptides comprising an RSV polypeptide and a ferritin protein.

This application is a divisional of U.S. application Ser. No. 17/061,065, filed Oct. 1, 2020, which is a continuation of International Application No. PCT/US2019/025387, filed Apr. 2, 2019, which claims the benefit of priority of U.S. Provisional Patent Application No. 62/652,199, filed Apr. 3, 2018, the entire contents of each of which are incorporated herein by reference.

The present application contains a Sequence Listing that has been submitted electronically in XML format and is hereby incorporated by reference in its entirety. Said XML copy, created on Jul. 8, 2024, is named 01121-0031-01US-ST26.xml and is 108,948 bytes in size.

Even with many successes in the field of vaccinology, new breakthroughs are needed to protect humans against many life-threatening infectious diseases. Many currently licensed vaccines rely on decade-old technologies to produce live-attenuated or inactivated killed pathogens, which carry inherent safety concerns and in many cases, stimulate only short-lived, weak immune responses that require the administration of multiple doses. While advances in genetic and biochemical engineering have made it possible to develop therapeutic agents to challenging disease targets, these applications to the field of vaccinology have not been fully realized. Recombinant protein technologies now allow the design of optimal antigens. Additionally, nanoparticles have increasingly demonstrated the potential for optimal antigen presentation and targeted drug delivery. Nanoparticles with multiple attached antigens have been shown to have increased binding avidity afforded by the multivalent display of their molecular cargos, and an ability to cross biological barriers more efficiently due to their nanoscopic size. Helicobacter pylori (H. pylori) ferritin nanoparticles fused to influenza virus haemagglutinin (HA) protein has allowed improved antigen stability and increased immunogenicity in mouse influenza models (see Kanekiyo et al., Nature 499:102-106 (2013)). This fusion protein self-assembled into an octahedrally-symmetric nanoparticle and presented 8 trimeric HA spikes to give a robust immune response in various pre-clinical models when used with an adjuvant.

Respiratory syncytial virus (RSV) is a leading cause of severe respiratory disease in infants and a major cause of respiratory illness in the elderly. It remains an unmet vaccine need despite decades of research. While the need for a vaccine is clear, development of an RSV vaccine was stymied in the 1960s when a clinical trial using a formalin inactivated RSV virus made disease, following RSV infection, more severe in infants. See, Hurwitz (2011) Expert Rev Vaccines 10(10): 1415-1433. More recently, clinical programs using an RSV F antigen in its post-fusion conformation failed to elicit sufficient efficacy in adults. See, Faloon et al. (2017) JID 216:1362-1370. However, RSV F antigens stabilized in the pre-fusion conformation may elicit a neutralizing response superior to that of the post-fusion antigens that have failed in the clinic.

Here, a set of new polypeptides, nanoparticles, compositions, methods, and uses involving RSV polypeptides is presented. Novel RSV F polypeptides were generated, including polypeptides in which an epitope of the RSV polypeptide that is shared between pre-fusion RSV F and post-fusion RSV F is blocked, e.g., by an N-glycan at a glycosylation site added by a mutation. Also generated were antigenic polypeptides and nanoparticles comprising these novel RSV polypeptides and ferritin. Antigenic ferritin polypeptides comprising RSV G polypeptides were also generated. Furthermore, self-adjuvanting antigenic polypeptides comprising RSV polypeptides and ferritin were developed wherein immune-stimulatory moieties, such as adjuvants, were directly, chemically attached to the antigenic polypeptide. The direct conjugation of an immune-stimulatory moiety to the antigenic polypeptide allows for targeted co-delivery of the immune-stimulatory moiety and RSV polypeptide in a single macromolecular entity, which can greatly decrease the potential for systemic toxicity that is feared with traditional vaccines that comprise antigens and immune-stimulatory molecules such as adjuvants as separate molecules. The co-delivery of immune-stimulatory moieties together with RSV polypeptides in a macromolecular entity and their multivalent presentation may also reduce the overall dose needed to elicit protection, reducing manufacturing burdens and costs.

### SUMMARY

It is an object of this disclosure to provide compositions, kits, methods, and uses that can provide one or more of the advantages discussed above, or at least provide the public with a useful choice. Accordingly, the following embodiments are disclosed herein.

Embodiment 1 is an antigenic RSV polypeptide comprising an RSV F polypeptide, wherein an epitope of the RSV polypeptide that is shared between pre-fusion RSV F and post-fusion RSV F is blocked.

Embodiment 2 is an antigenic RSV polypeptide comprising an RSV F polypeptide, wherein the RSV F polypeptide comprises amino acid residues 62-69 and 196-209 of SEQ ID NO: 26 and an asparagine corresponding to position 328, 348, or 507 of SEQ ID NO: 26.

Embodiment 3 is the antigenic RSV polypeptide of embodiment 2, wherein an epitope of the RSV polypeptide that is shared between pre-fusion RSV F and post-fusion RSV F is blocked.

Embodiment 3b is the antigenic RSV polypeptide of embodiment 1 or 3, wherein the blocked epitope is an epitope of antigenic site 1 of RSV F.

Embodiment 3c is the antigenic RSV polypeptide of embodiment 1 or 3-3b, wherein two or more epitopes shared between pre-fusion RSV F and post-fusion RSV F are blocked.

Embodiment 3d is the antigenic RSV polypeptide of embodiment 1 or 3-3c, wherein two or more epitopes of antigenic site 1 of RSV F are blocked.

Embodiment 3e is the antigenic RSV polypeptide of embodiment 1 or 3-3d, wherein one or more, or all, epitopes that topologically overlap with the blocked epitope are also blocked.

Embodiment 3f is the antigenic RSV polypeptide of embodiment 3e, wherein the blocked epitope is an epitope of antigenic site 1 of RSV F.

Embodiment 4 is the antigenic RSV polypeptide of any one of the preceding embodiments, comprising a pre-fusion RSV F.

Embodiment 5 is the antigenic RSV polypeptide of any one of the preceding embodiments, which is recognized by a pre-fusion RSV F-specific antibody selected from D25 or AM14.

Embodiment 6 is the antigenic RSV polypeptide of embodiment 4 or 5, wherein the pre-fusion RSV F comprises an epitope not found on post-fusion RSV F.

Embodiment 7 is the antigenic RSV polypeptide of any one of embodiments 1-3, comprising a post-fusion RSV F.

Embodiment 8 is the antigenic RSV polypeptide of any one of embodiments 1 or 3-6, wherein the epitope is blocked with an N-glycan attached to asparagine.

Embodiment 9 is the antigenic RSV polypeptide of embodiment 7, wherein the asparagine corresponds to a non-asparagine residue in a wild-type RSV F sequence (SEQ ID NO: 26), optionally wherein the non-asparagine residue corresponds to position 328, 348, or 507 of SEQ ID NO: 26.

Embodiment 10 is the antigenic RSV polypeptide of any one of the preceding embodiments, further comprising a ferritin protein.

Embodiment 11 is the antigenic RSV polypeptide of embodiment 11, wherein the ferritin comprises a mutation replacing a surface-exposed amino acid with a cysteine.

Embodiment 12 is an antigenic RSV polypeptide comprising an RSV F polypeptide and a ferritin protein, wherein the ferritin protein comprises a mutation replacing a surface exposed amino acid with a cysteine.

Embodiment 13 is the antigenic RSV polypeptide of any one of embodiments 11-12, wherein the ferritin comprises one or more of E12C, S26C, S72C, A75C, K79C, S100C, and S111C mutations of H. pylori ferritin or one or more corresponding mutations in a non-H. pylori ferritin as determined by pairwise or structural alignment.

Embodiment 14 is the antigenic RSV polypeptide of any one of embodiments 10-13, comprising one or more immune-stimulatory moieties linked to the ferritin via a surface-exposed amino acid, optionally wherein the surface-exposed amino acid is a cysteine resulting from a mutation.

Embodiment 15 is the antigenic RSV polypeptide of any one of embodiments 10-14, wherein the ferritin comprises a mutation replacing a surface-exposed asparagine with a non-asparagine amino acid, optionally wherein the asparagine is at position 19 of H. pylori ferritin, or an analogous position in a non-H. pylori ferritin as determined by pairwise or structural alignment.

Embodiment 16 is the antigenic RSV polypeptide of any one of embodiments 10-15, wherein the ferritin comprises a mutation replacing an internal cysteine with a non-cysteine amino acid, optionally wherein the internal cysteine is at position 31 of H. pylori ferritin, or a position that corresponds to position 31 of H. pylori ferritin as determined by pair-wise or structural alignment.

Embodiment 17 is the antigenic RSV polypeptide of any one of embodiments 12-16, wherein the RSV F polypeptide comprises an epitope not found on post-fusion RSV F which is a site 0 epitope, optionally wherein the site 0 epitope comprises amino acid residues 62-69 and 196-209 of SEQ ID NO: 26.

Embodiment 18 is the antigenic RSV polypeptide of any one of the preceding embodiments, wherein the RSV F polypeptide comprises an asparagine at a position corresponding to position 328 of SEQ ID NO: 26.

Embodiment 19 is the antigenic RSV polypeptide of any one of the preceding embodiments, wherein the RSV F polypeptide comprises an asparagine at a position corresponding to position 348 of SEQ ID NO: 26.

Embodiment 20 is the antigenic RSV polypeptide of any one of the preceding embodiments, wherein the RSV F polypeptide comprises an asparagine at a position corresponding to position 507 of SEQ ID NO: 26.

Embodiment 21 is the antigenic RSV polypeptide of any one of the preceding embodiments, wherein RSV F polypeptide comprises a leucine at a position corresponding to position lysine 498 of SEQ ID NO: 26.

Embodiment 22 is the antigenic RSV polypeptide of any one of the preceding embodiments, wherein the RSV F polypeptide comprises a proline at a position corresponding to position isoleucine 217 of SEQ ID NO: 26.

Embodiment 23 is the antigenic RSV polypeptide of any one of the preceding embodiments, wherein the RSV F polypeptide comprises an amino acid other than cysteine at a position corresponding to position 155 of SEQ ID NO: 26 and/or an amino acid other than cysteine at position corresponding to position 290 of SEQ ID NO: 26.

Embodiment 24 is the antigenic RSV polypeptide of any one of the preceding embodiments, comprising a serine at a position corresponding to position 155 of SEQ ID NO: 26 and/or a serine at a position corresponding to position 290 of SEQ ID NO: 26.

Embodiment 25 is the antigenic RSV polypeptide of any one of the preceding embodiments, wherein the RSV F polypeptide lacks a furin cleavage site, optionally wherein a linker is present in place of the furin cleavage site.

Embodiment 26 is the antigenic RSV polypeptide of any one of the preceding embodiments, wherein the RSV F polypeptide comprises a sequence having at least 85%, 90%, 95%, 97%, 98%, 99%, or 99.5% identity to amino acids 1-478 of SEQ ID NO: 17.

Embodiment 27 is the antigenic RSV polypeptide of any one of the preceding embodiments, wherein the RSV F polypeptide comprises a sequence having at least 85%, 90%, 95%, 97%, 98%, 99%, or 99.5% identity to the sequence of SEQ ID NO: 17.

Embodiment 28 is the antigenic RSV polypeptide of embodiment 20 or 21, comprising amino acids 1-478 of SEQ ID NO: 17.

Embodiment 29 is the antigenic RSV polypeptide of any one of embodiments 1-19, wherein the RSV F polypeptide comprises a sequence having at least 85%, 90%, 95%, 97%, 98%, 99%, or 99.5% identity to amino acids 1-478 of SEQ ID NO: 23.

Embodiment 30 is the antigenic RSV polypeptide of any one of embodiments 1-19 or 23, wherein the RSV F polypeptide comprises a sequence having at least 85%, 90%, 95%, 97%, 98%, 99%, or 99.5% identity to the sequence of SEQ ID NO: 23.

Embodiment 31 is the antigenic RSV polypeptide of embodiment 23 or 24, comprising amino acids 1-478 of SEQ ID NO: 23.

Embodiment 32 is the antigenic RSV polypeptide of any one of the preceding embodiments, comprising the sequence of any one of SEQ ID NOs: 3-23.

Embodiment 32a is the antigenic RSV polypeptide of claim 32, comprising the sequence of SEQ ID NO: 17.

Embodiment 32b is the antigenic RSV polypeptide of claim 32, comprising the sequence of SEQ ID NO: 23.

Embodiment 33 is a ferritin particle comprising the antigenic RSV polypeptide of any one of embodiments 10-32b.

Embodiment 34 is a composition comprising the antigenic RSV polypeptide or ferritin particle of any one of the preceding embodiments and an RSV G polypeptide.

Embodiment 34b is the composition of embodiment 34, wherein the composition comprises the ferritin particle, and the ferritin particle comprises the RSV G polypeptide, optionally wherein the RSV G polypeptide is chemically conjugated to the ferritin particle.

Embodiment 34c is the composition of embodiment 34 or 34b, wherein the RSV G polypeptide is not glycosylated.

Embodiment 35 is a composition comprising the antigenic RSV polypeptide or ferritin particle of any one of embodiments 1-33, or the composition of any one of embodiments 34-34c, further comprising a pharmaceutically acceptable carrier.

Embodiment 36 is the antigenic RSV polypeptide, ferritin particle, or composition of any one of embodiments 1-35 for use in a method of eliciting an immune response to RSV or in protecting a subject against RSV infection.

Embodiment 37 is a method of eliciting an immune response to RSV or protecting a subject against RSV infection comprising administering any one or more of the antigenic RSV polypeptide, ferritin particle, or composition of any one of embodiments 1-36 to a subject.

Embodiment 38 is the antigenic RSV polypeptide, ferritin particle, composition, or method of any one of embodiments 36-37, wherein the subject is human.

Embodiment 39 is a nucleic acid encoding the antigenic RSV polypeptide of any one of embodiments 1-32b, optionally wherein the nucleic acid is an mRNA.

Embodiment 39b is a composition or kit comprising the nucleic acid of embodiment 39 and a nucleic acid encoding an RSV G polypeptide, optionally wherein one or both nucleic acids are mRNAs.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate several embodiments and together with the description, serve to explain the principles described herein.

### BRIEF DESCRIPTION OF THE DRAWINGS

FIGS. 1A-1D show an exemplary RSV Pre-F-NP polypeptide structure. (FIG. 1A) Linear diagram listing residue numbers corresponding to the N terminus of each segment. Numbering is according to SEQ ID NO: 26. Domains 1-3 are indicated with DI, DII and DIII, respectively, and heptad repeat region A (HRA) and heptad repeat region B (HRB) are also labeled. The C-terminal ferritin is labeled (Ferritin Nanoparticle). The F1 and F2 fragments of the RSV F moiety are labeled below the cartoon. The region between the F1 and F2 fragment, where the peptide 27 fragment (p27) fusion peptide (FP) and furin cleavage sites (furin sites) were deleted and replaced with a flexible linker to form single chain F constructs, is depicted as a line and labeled above the cartoon. Stars above the diagram indicate approximate locations of engineered glycosylation sites E328N, S348N and R507N. (FIG. 1B) Structural model of pre-fusion RSV F moiety indicating key neutralizing (Nab) epitopes for D25, AM14, 101F, and Palivizumab antibodies. The approximate region of shared pre-fusion and post-fusion structural epitopes is indicated with a white triangle. The locations of exemplary engineered glycosylation sites E328N, S348N and R507N are labeled. The engineered glycosylation sites map to regions structurally shared between pre-fusion and post-fusion conformations and away from key neutralizing epitopes recognized by antibodies such as D25, AM14, 10IF and Palivizumab. As such, constructs containing these engineered glycan sites still bind to the above neutralizing antibodies (data not shown). (FIG. 1C) Structural model of RSV pre-fusion F protein nanoparticle (Pre-F-NP) with HRA and HRB regions shaded darker. The resulting folded Pre-F-NP constructs can form 24-mers that display the key epitopes listed in FIG. 1B. (FIG. 1D) 2D class averages of electron micrographs of RSV Pre-F-NP construct RF8085 (SEQ ID NO: 1) showing symmetry of RSV F trimer moieties on the 24-mer ferritin nanoparticle.

FIG. 2 shows small-scale expression of several Pre-F-NP constructs expressed in 293 cell conditioned media as measured by D25 antibody Western blot. RF8090 is SEQ ID NO: 2, which is a cloning variant used in CHO expression having the same sequence as RF8085, i.e., SEQ ID NO:1. RF8085 and RF8090 are Pre-F-NP constructs harboring the disulfide and cavity filling mutations of DS-CAV with the deletions and single chain linker described in FIG. 1A fused N-terminally to ferritin. RF8100-RF8105 and RF8108-RF8112 have the sequences of SEQ ID NOs: 3-8 and 11-14, respectively. scF-pFerr=fusion protein of RSV F polypeptide and ferritin. Mutations that appear to improve expression of the construct relative to the RF8090 benchmark are indicated below the Western blot. Notable mutations include the addition of glycan sites via the E328N, S348N and R507N mutations and the central helix capping mutation I327P, which increased expression and secretion of the RSV F nanoparticle into the conditioned media as measured by Western blot.

FIG. 3 shows expression of RF8085 (SEQ ID NO: 1; control construct) and RF8106 (SEQ ID NO: 9; comprising a I217P mutation as in RF8108 and lacking the disulfide (DS) mutation of DS-CAV1) as measured by the Western blot analysis of conditioned media from 293 expression. Replacing the DS with the central helix capping mutation I217P increased expression significantly. Replacing the DS with the central helix capping mutation does not affect binding of the construct to pre-fusion-specific antibodies D25 and AM14.

FIG. 4 shows results of size exclusion chromatography purification of the RF8106 construct (SEQ ID NO: 9). The retention volume of the RF8106 nanoparticle of approximately 65 ml on the Superose 6 preparatory SEC column is consistent with a folded 24-mer nanoparticle, suggesting the mutations in RF8106 did not hinder nanoparticle formation.

FIGS. 5A-5B show dynamic light scattering (DLS) analysis of nonreduced (5A) and reduced (5B) RF8106. Like the SEC analysis, the DLS demonstrates that the RSV Pre-F-NP formed the expected, folded nanoparticle. The reduced data further show that the particle was not disrupted by reduction, which was performed before adjuvant conjugation to the surface-exposed cysteine introduced on ferritin by a mutation (see FIG. 6).

FIG. 6 shows a coomassie-stained SDS-PAGE gel analysis of RF8106 with and without conjugation to the TLR9 agonist CpG. The increased gel shift of the CpG-treated nanoparticle demonstrated that the CpG adjuvant can be added to the RSV F nanoparticle to approximately 40-50% completion. Conjugation of CpG or other immune-stimulatory moieties such as TLR7/8 agonist SM7/8 did not inhibit the particle's ability to bind pre-fusion specific antibodies D25 and AM14.

FIG. 7 shows Western blot of nanoparticles comprising RSV F with (RF8117, SEQ ID NO: 17) and without additional glycans (RF8085, SEQ ID NO: 1; and RF8113, SEQ ID NO: 16). RF8113 is like RF8106, but the S111C surface-exposed cysteine (using ferritin residue numbering, i.e., corresponding to positions in the ferritin sequence of SEQ ID NO: 208) from RF8106 has been replaced with a K79C surface-exposed cysteine (also using ferritin residue numbering) to place the conjugation site further from the Pre-F moiety. Like RF8106, RF8113 retains improved expression over the benchmark molecule RF8085. RF8117 is like RF8113 but further comprises the three glycosylation mutations identified in FIG. 2, i.e. E328N, S348N and R507N, to further improve expression and block the non-neutralizing epitopes shared between the Pre-fusion F and Post-fusion F conformations as described in FIG. 1B.

FIG. 8 shows expression of RSV F constructs with different substitutions at potential trypsin-like protease cleavage sites. It was observed in CHO cell line expression of RF8090 (same protein sequence as RF8085 with a different DNA sequence adapted to the CHO expression vector) that the polypeptide was clipped between the F and ferritin moiety, resulting in reduced expression. By the resulting masses of the F moiety, it was estimated that proteolysis could be taking place near the HRB, bull-frog linker region of the Pre-F-NP construct. Mutations of lysine and arginine residues within this region (residues ˜450-550) were explored to eliminate potential trypsin-like proteolysis of the construct. The mutations in RF8122 (SEQ ID NO: 18) relative to RF8117 (K498L and K508Q) provided improved expression in 293 cells and may reduce or eliminate proteolysis in CHO cells. Alternative mutations limited expression.

FIGS. 9A-B. Expression of RF8090, RF8117 and RF8140 in stably transfected CHO cells. Expression yield of RF8090 (SEQ ID NO: 2) was observed at low levels. Mutations to replace the disulfide of DS-CAV1 and mutations to the linker between the F moiety and ferritin moiety to eliminate potential typsin cleavage sites were introduced as described above to constructs RF8117 (SEQ ID NO: 17) and RF8140 (SEQ ID NO: 23), which were cloned into stably expressing CHO cells. (FIG. 9A) Expression of RF8117 and RF8140 from three and four pools of CHO cells, respectively, into CHO conditioned media was compared to yields of RF8090 in CHO conditioned media by D25-Western blot analysis. All three CHO pools for RF8117 and all four CHO pools for RF8140 express to higher yields than RF8090. (FIG. 9B) Expression of RF8117 into CHO conditioned media as measured by D25 pre-fusion F-specific antibody by Octet. The left panel shows response of RF8140 purified from 293 media of known concentrations plotted against response of binding to D25 on a Protein A tip providing a standard curve. Individual dots represent responses to D25 binding from RF8117 CHO conditioned media. The right panel shows calculated yield of RF8117 or RF8140 in CHO pool conditioned media based on D25 binding response. Both RF8117 and RF8140 were expressed in the media as measured by D25 and AM14 binding, demonstrating that like 293 cells, CHO cells are able to express the Pre-F-NPs in a folded manner which retains the pre-fusion F trimer structure.

FIGS. 10A-B. Neutralizing antibody response to Pre-F-NP RF8117. (FIG. 10A) Comparison of RSV neutralizing titers elicited by High Dose (1 μg) and Low Dose (0.1 μg) immunization of DS-CAV1 (Pre-F Trimer, SEQ ID NO: 25), Post-fusion F Trimer (Post-F Trimer; SEQ ID NO: 24) or Pre-F-NP with engineered glycosylation (Pre-F-NP; RF8117, SEQ ID NO: 17) was measured by VERO cell assay. All RSV polypeptides were administered with adjuvant AF03 as described herein. Throughout, unless states otherwise, AF03 was administered with the RSV polypeptide or nanoparticle, but not conjugated to it. RSV polypeptides and doses are labeled below the x-axis. Statistical analysis of high dose responses relative to Pre-F-NP immunization is indicated. (FIG. 10B) Comparison of RSV neutralizing titers elicited by High Dose (1 μg) and Low Dose (0.1 μg) immunization with DS-CAV1 (Pre-F Trimer), Pre-F-NP without engineered glycosylation (RF8113, SEQ ID NO: 16) or Pre-F-NP with engineered glycosylation (RF8117, SEQ ID NO: 17) as measured by VERO cell assay. All RSV polypeptides were administered with adjuvant AF03 (not conjugated to any polypeptide or nanoparticle) as described herein. RSV polypeptides and doses are labeled below the x-axis.

FIG. 11A-D. Comparison of RSV pre-fusion F trimer (DS-CAV1) binding antibody and RSV neutralizing antibodies elicited by immunization with post-fusion F trimer (SEQ ID NO: 24) or Pre-F-NP (RF8140 SEQ ID NO: 23) in mouse or non-human primate models. (FIG. 11A) Pre-fusion F trimer binding antibody responses elicited in mice from immunization between post-fusion F and Pre-F-NP (RF8140, SEQ ID NO: 23) are compared. (FIG. 11B) Neutralizing antibody responses elicited in mice from immunization with post-fusion F and Pre-F-NP (RF8140, SEQ ID NO: 23) are shown. (FIG. 11C) Pre-fusion F trimer binding antibody responses elicited in non-human primates by Pre-F-NP with or without adjuvant (AF03, indicated in parentheses below) are compared. (FIG. 11D) RSV neutralizing titers elicited by immunization with Pre-F-NP (RF8140, SEQ ID NO: 23) with and without AF03 adjuvant are compared. In mice, Pre-F-NP elicits a higher pre-fusion F binding response and RSV neutralizing response compared to post-fusion trimer. In non-human primates, Pre-F-NP elicits a potent neutralizing response.

FIGS. 12A-12B show that engineered glycosylation sites block post-fusion epitopes. (FIG. 12A) Antibody response to pre-fusion F (DS-CAV1) elicited by immunization with Pre-F-NP without engineered glycosylation (RF8113) or Pre-F-NP with engineered glycosylation (Engineered Gly Particle) at high (1 μg) and low (0.1 μg) dose as measured by Octet is shown. (FIG. 12B) Antibody response to post-fusion trimer elicited by immunization with Pre-F-NP without engineered glycosylation (RF8113) or Pre-F-NP with engineered glycosylation (RF8117) at high (1 μg) and low (0.1 μg) dose as measured by Octet is shown. As above, all RSV polypeptides were mixed with AF03 during immunization. While both RF8113 and RF8117 elicit robust antibody responses to pre-fusion F, the post-fusion F antibody response elicited by RF8117 is greatly reduced. This is due to the engineered glycans mapping to the shared pre-fusion and post-fusion epitopes (FIG. 2B).

FIGS. 13A-C show blocking of non-neutralizing epitopes by engineered glycosylation sites. (FIG. 13A) Comparison of RSV neutralizing titers elicited by immunization with Pre-F NP with wild-type glycosylation sites (“Wt Glycan Particle”; RF8113, SEQ ID NO: 16) versus Pre-F NP with additional engineered glycosylation sites (“+Glycan Particle”; RF8117, SEQ ID NO: 17) at 0.1 μg dose in mouse studies as measured by VERO cell assay. (FIG. 13B) Comparison of RSV Post-fusion F trimer-binding antibody responses elicited by immunization with Wt Glycan Particle (RF8113, SEQ ID NO: 16) versus +Glycan Particle (RF8117, SEQ ID NO: 17) at 0.1 μg dose in mouse studies. (FIG. 13C) Ratio of measured neutralization titers to binding titers from panels A and B demonstrating that the engineered glycans did not reduce the functional, neutralizing antibody response but did decrease the non-neutralizing antibodies elicited to the shared pre-fusion/post-fusion epitopes (FIG. 1i), thus improving the Neutralizing/Binding antibody ratio.

FIG. 14A-D. Characterization of RSV G central domain peptide (Gcc) conjugated to ferritin nanoparticle. (FIG. 14A) Coomassie-stained SDS-PAGE gel showing the click-conjugation of RSV G central domain (SEQ ID NO. 29) to ferritin nanoparticle, forming the Gcc-NP antigen. (FIG. 14B) Structural model of Gcc-NP. (FIG. 14C) Comparison of Gcc-binding antibody responses elicited by immunization with Gcc peptide alone (Gcc peptide, SEQ ID NO. 29) versus Gcc peptide conjugated to nanoparticle (Gcc-NP) in mouse studies. A representative response from naïve sera is shown in white box, while responses from post-second immunization are shown in light grey boxes and responses from post-third immunizations are shown in dark grey boxes. (FIG. 14D) Comparison of RSV neutralizing titers elicited by immunization with Gcc peptide (SEQ ID NO. 29) versus Gcc-NP in mouse studies post-third injection as measured by HAE cell assay. Sera from naïve animals and sera from animals immunized with Gcc peptide were pooled and titers are shown as bars.

FIGS. 15A-C. Co-administration of RSV Pre-F-NP (RF8140) and Gcc-NP elicit a neutralizing response. Mice were immunized with Pre-F-NP (RF8140) alone, Gcc-NP alone, or Pre-F-NP and Gcc-NP combined at 1 μg dose per antigen. All immunizations were adjuvanted with AF03 as above. (FIG. 15A) Immunization of mice with RF8140 alone (Pre-F-NP) or RF8140 and Gcc-NP (Pre-F-NP+Gcc-NP) elicited antibodies that bind pre-fusion F trimer. (FIG. 15B) Immunization of mice with Gcc-NP alone (Gcc-NP) or RF8140 and Gcc-NP (Pre-F-NO+Gcc-NP) elicited antibodies that bind Gcc peptide. (FIG. 15C) Animals immunized with either Pre-F-NP alone, Gcc-NP alone, or the co-administration of Pre-F-NP and Gcc-NP elicit a neutralizing response post-second and post-third immunization as measured by HAE neutralizing assay. Co-administration of Pre-F-NP+Gcc-NP elicited a neutralizing response superior to that elicited by immunization with only Pre-F-NP.

FIG. 16A-B. Co-administration of Pre-F-NP and Gcc-NP does not interfere with elicitation of antibodies that bind Pre-fusion F trimer or Gcc-nanoparticle. Neutralizing titers measured by the F-sensitive VERO cell assay are on the left in FIG. 16A, while neutralizing titers measured by the F- and G-sensitive HAE assay are shown on the right in FIG. 16B. Animal immunizations were as in FIG. 15. RSV polypeptides used in the immunization are below the horizontal axis. The black bars represent sera pooled from the immunization groups described in FIG. 15 and are similarly labeled. Sera from naïve animals are also shown as black bars and labeled for comparison. Sera depleted with pre-fusion F trimer are in white, just to the right of the corresponding black bar. Sera depleted with G ectodomain are in diagonally striped bars, just to the right of the corresponding black bar. Sera depleted with pre-fusion F trimer followed by depletion with G ectodomain is in a vertically striped bar. (FIG. 16A) Neutralizing titers were observed in VERO cell assays for sera from RF8140 immunization and RF8140+Gcc-NP co-administration, but not naïve sera or sera from Gcc-NP immunization alone. Depletion of sera from RF8140 or RF8140+Gcc-NP groups with pre-fusion F trimer reduced the measurable neutralizing titers. (FIG. 16B) Neutralizing titers were observed in HAE cell assays for sera from animals immunized with RF8140, Gcc-NP, or RF8140 co-administered with Gcc-NP. Sera from naïve animals did not have a neutralizing response. Sera from animals immunized with RF8140 that is depleted with pre-fusion F trimer has a reduction in measurable neutralizing titer. Sera from animals immunized with Gcc-NP that is depleted with G ectodomain has a reduction in measurable neutralizing titer. Sera from animals immunized with a co-administration of RF8140 and Gcc-NP does not have a reduced measurable neutralizing titer when depleted with pre-fusion F trimer alone, but does have a reduced measurable neutralizing titer when depleted with both pre-fusion F trimer and G ectodomain. Together, these data suggest co-administration with the Pre-F-NP and Gcc-NP does not interfere with the antigens' respective abilities to elicit neutralizing antibodies to pre-fusion F or G.

FIGS. 17A-B. Adjuvanting RF8117 or RF8140 with AF03, SPA09 or Alum elicits a superior neutralizing response in mice relative to unadjuvanted RF8117. (FIG. 17A) Neutralizing titers for sera from mice immunized with RF8117 either unadjuvanted (No Adj), adjuvanted with Alum, or adjuvanted with AF03 are shown as measured by VERO cell assay. (FIG. 17B) Neutralizing titers for sera from mice immunized with RF8117 either unadjuvanted (No Adj), RF8117 adjuvanted with SPA09, or RF8140 adjuvanted with AF03 are shown as measured by VERO cell assay. In all cases for either RF8117 or RF8140, in naïve mice adjuvanted groups elicited a higher neutralizing titer than non-adjuvanted groups.

FIGS. 18A-B. Adjuvanting RF8140 with AF03 or SPA09 elicits a superior neutralizing response in non-human primates (NHPs) relative to unadjuvanted RF8140 immunizations. (FIG. 18A) Pre-fusion F trimer responses measured in NHP sera after immunization with RF8140 either unadjuvanted (No Adj), adjuvanted with AF03 or adjuvanted with SPA09 (two doses of SPA09 were used, as indicated below) as measured by ELISA. At all timepoints, adjuvanting with AF03 or SPA09 elicits a superior neutralizing response. (FIG. 18B) Neutralizing titers for sera from NHPs immunized with RF8140 either unadjuvanted (No Adj), adjuvanted with AF03 or adjuvanted with SPA09 (two doses of SPA09 were used, as indicated below) as measured by VERO cell assay. In all cases immunization with RF8140 with adjuvant elicits a higher neutralizing titer than non-adjuvanted groups at all timepoints.

FIGS. 19A-B. Conjugation of RF8140 to TLR7/8 agonist SM7/8 or TLR9 agonist CpG elicits a superior pre-fusion F-binding titer relative to unadjuvanted RF8140 alone. (FIG. 19A) Pre-fusion F trimer-binding response measured in sera from either naïve mice, mice immunized with unadjuvanted RF8140, mice immunized with RF8140 conjugated with SM7/8 adjuvant, RF8140 adjuvanted with 130 ng of SM7/8 or RF8140 adjuvanted with 20 μg SM7/8 are shown. RF8140 conjugated to SM7/8 elicits a higher pre-fusion F trimer-binding titer than unadjuvanted or SM7/8 adjuvanted groups. (FIG. 19B) Pre-fusion F trimer-binding response measured in sera from either naïve mice, mice immunized with unadjuvanted RF8140, mice immunized with RF8140 conjugated with CpG adjuvant, RF8140 adjuvanted with 680 ng of CpG or RF8140 adjuvanted with 20 μg SM7/8 are shown. RF8140 conjugated to SM7/8 elicits a higher pre-fusion F trimer-binding titer than unadjuvanted or SM7/8 adjuvanted groups.

FIGS. 20A-G. F-subunit vaccine candidates elicit pre-F directed neutralizing antibodies and a Th1 CD4⁺ T cell response in the MIMIC system. (FIG. 20A) Anti-pre-F titers in MIMIC system were measured by AF after priming with each Ag at molar equivalent concentration of F with 10 ng/ml of pre-F NP (n=48-49 donors per group). (FIG. 20B) Microneutralization titers were measured and are represented in International units/ml (IU/ml). (FIG. 20C) A ratio between anti-pre-F and post-F>1 represents a higher level of pre-F-binding antibody versus post-F-binding antibody while a ratio value<1 represents a greater Ab response to post-F. (FIG. 20D) The production of TNFα in activated CD154⁺/CD4⁺ T cells re-stimulated with F protein loaded target cells was measured using flow cytometry, n=48. Statistical significance was determined via Tukey-Kramer-HSD multiple comparison (FIG. 20E) Pre-existing antibody titer in humans subjects (serostatus) is strongly correlated with the magnitude of the RSV immune response in MIMIC system. Linear regression plot showing anti-pre-F IgG in sera from each donor versus total anti-pre-F IgG response was generated by software or algorithm and thep value for the common slope was analyzed by statistics method (n=50). Y-axis represents anti-pre-F IgG levels obtained following priming with RSV. (FIG. 20 F) As in FIG. 20E, linear regression plot showing anti-pre-F IgG in sera from each donor versus total anti-pre-F IgG after priming with F subunit vaccine candidates (post-F in squares, pre-F-NP in circles and DC-Cavl in diamonds). The anti-pre-F IgG pre-existing circulating titers ranged from 199,800 to 3,037,600,000. Each dot represents the IgG value of each individual donor. (FIG. 20G) Comparison of Gcc-binding antibody responses elicited by treatment with Gcc peptide alone (Gcc peptide) versus Gcc peptide conjugated to nanoparticle (Gcc-NP) in human B-cells. A no treatment group is shown for comparison as above.

FIGS. 21A-C. Neutralizing antibody titers elicited by a low dose (0.5 μg) of RSV Gcc-ferritin nanoparticles (“Gcc-NP”). Shown are RSV A strain HAE neutralizing titers elicited from immunization with RSV Gcc-NP containing the RSV A2 Gcc sequence (formulated with AF03), from sera taken two weeks post the second immunization (2wp2) (FIG. 21A) or two weeks post the third immunization (2wp3) (FIG. 21B), with naïve and hyperimmune sera as negative and positive controls. Also shown is an RSV B strain HAE neutralizing titer elicited from immunization with RSV Gcc-NP containing the RSV A2 Gcc sequence (formulated with AF03), from sera taken two weeks post the third immunization (2wp3) (FIG. 21C).

FIGS. 22A-B. RSV A2 strain antigen-binding antibody responses elicited by RSV Gcc-NP. (FIG. 22A) Gcc-binding antibody responses elicited to the Gcc A2 strain measured at two weeks post the second injection (light grey boxes) and two weeks post the third injection (dark grey boxes) elicited by the high dose (5 μg) of RSV Gcc-NP. Naïve mouse sera response is shown as a negative control. (FIG. 22B) Gcc-binding antibody responses elicited to the Gcc A2 strain measured at two weeks post the second injection (light grey boxes) and two weeks post the third injection (dark grey boxes) elicited by the low dose (0.5 μg) of RSV Gcc-NP.

FIGS. 23A-B. RSV B1 strain antigen-binding antibody responses elicited by RSV Gcc-NP. (FIG. 23A) Gcc-binding antibody responses elicited to the Gcc B1 strain measured at two weeks post the second injection (light grey boxes) and two weeks post the third injection (dark grey boxes) elicited by a high dose (5 μg) of RSV Gcc-NP. Naïve mouse sera response is shown as a negative control. (FIG. 23B) Gcc-binding antibody responses elicited to the Gcc B1 strain measured at two weeks post the second injection (light grey boxes) and two weeks post the third injection (dark grey boxes) elicited by a low dose (0.5 μg) of RSV Gcc-NP.

### DETAILED DESCRIPTION

RSV polypeptides are provided, which can be antigenic when administered alone, with adjuvant as a separate molecule, and/or as part of a nanoparticle (e.g., ferritin particle or lumazine synthase particle), which can be self-adjuvanting. In some embodiments, the antigenic RSV polypeptides comprise an RSV F polypeptide and a ferritin, and/or an RSV F polypeptide in which an epitope of the RSV polypeptide that is shared between pre-fusion RSV F and post-fusion RSV F is blocked. RSV F polypeptides that direct production of antibodies against the pre-fusion conformation of RSV F induced higher in vivo antibody response to pre-fusion RSV F in comparison to post-fusion RSV F. Also described herein are RSV G polypeptides comprising all or part of RSV G, and can further comprise a ferritin. The RSV G and RSV F proteins are essential for attachment and fusion of RSV to host cells.

RSV F exists in two conformational states, the pre-fusion and post-fusion conformations. In its native pre-fusion state, RSV F is a trimer comprised of 3 protomers. Thus, immunization with RSV F polypeptides in the pre-fusion conformation may have improved properties. In some embodiments, the RSV F polypeptide is designed to induce immunity against RSV F in the pre-fusion conformation. RSV G is an attachment protein responsible for associating RSV with human airway epithelial cells.

### A. Definitions

“Antigenic site 0” or “site 0 epitope,” as used herein, refer to a site located at the apex of the pre-fusion RSV F trimer, comprising amino acid residues 62-69 and 196-209 of wild-type RSV F (SEQ ID NO: 26). The site 0 epitope is a binding site for antibodies that have specificity for pre-fusion RSV F, such as D25 and AM14, and binding of antibodies to the site 0 epitope blocks cell-surface attachment of RSV (see McLellan et al., Science 340(6136):1113-1117 (2013)).

“Antigen stability,” as used herein, refers to stability of the antigen over time or in solution.

“Cavity filling substitutions,” as used herein, refers to engineered hydrophobic substitutions to fill cavities present in the pre-fusion RSV F trimer.

“F protein,” or “RSV F protein” refers to the protein of RSV responsible for driving fusion of the viral envelope with host cell membrane during viral entry.

“RSV F polypeptide” or “F polypeptide” refers to a polypeptide comprising at least one epitope of F protein.

“Glycan addition,” as used herein, refers to the addition of mutations which introduce glycosylation sites not present in wild-type RSV F, which can be engineered to increase construct expression, increase construct stability, or block epitopes shared between the pre-fusion and post-fusion confirmation. A modified protein comprising glycan additions would have more glycosylation and therefore a higher molecular weight. Glycan addition of can reduce the extent to which an RSV F polypeptide elicits antibodies to the post-fusion conformation of RSV F.

“G protein” or “RSV G protein” as used herein, refers to the attachment protein responsible for associating RSV with human airway epithelial cells. An exemplary wild-type RSV G amino acid sequence is provided as SEQ ID NO: 27. RSV G protein comprises an ectodomain (approximately amino acids 66-297 of RSV G (SEQ ID NO: 27)) that resides extracellularly. Within the ectodomain of RSV G is a central conserved region (Gcc or CCR, approximately amino acids 151-193 of SEQ ID NO: 27). The CCR of RSV G comprises a CX3C motif The CX3C motif mediates binding of G protein to the CX3CR1 receptor.

“Helix PRO capping” or “helix proline capping,” as used herein, refer to when a helix cap comprises a proline, which can stabilize helix formation.

“Intra-protomer stabilizing substitutions,” as used herein, describe amino acid substitutions in RSV F that stabilize the pre-fusion conformation by stabilizing the interaction within a protomer of the RSV F trimer.

“Inter-protomer stabilizing substitutions,” as used herein, describe amino acid substitutions in RSV F that stabilize the pre-fusion conformation by stabilizing the interaction of the protomers of the RSV F trimer with each other.

“Protease cleavage” as used herein, refers to proteolysis (sometimes also referred to as “clipping” in the art) of susceptible residues (e.g., lysine or arginine) in a polypeptide sequence.

“Post-fusion,” as used herein with respect to RSV F, refers to a stable conformation of RSV F that occurs after merging of the virus and cell membranes.

“Pre-fusion,” as used herein with respect to RSV F, refers to a conformation of RSV F that is adopted before virus-cell interaction.

“Protomer,” as used herein, refers to a structural unit of an oligomeric protein. In the case of RSV F, an individual unit of the RSV F trimer is a protomer.

“Ferritin” or “ferritin protein,” as used herein, refers to a protein with detectable sequence identity to H. pylori ferritin (SEQ ID NO: 208 or 209) or another ferritin discussed herein, such as P. furiosus ferritin, Trichoplusia ni ferritin, or human ferritin, that serves to store iron, e.g., intracellularly or in tissues or to carry iron in the bloodstream. Such exemplary ferritins, including those that occur as two polypeptide chains, known as the heavy and light chains (e.g., T. ni and human ferritin), are discussed in detail below. In some embodiments, a ferritin comprises a sequence with at least 15%, 20%, 30%, 40%, 50%, 60%, 70%, 75%, 80%, 85%, 90%, 95%, 97%, 98%, 99%, or 99.5% identity to a ferritin sequence disclosed herein, e.g., in Table 1 (Sequence Table). A ferritin may be a fragment of a full-length naturally-occurring sequence.

“Wild-type ferritin,” as used herein, refers to a ferritin whose sequence consists of a naturally-occurring sequence. Ferritins also include full-length ferritin or a fragment of ferritin with one or more differences in its amino acid sequence from a wild-type ferritin.

As used herein, a “ferritin monomer” refers to a single ferritin molecule (or, where applicable, a single ferritin heavy or light chain) that has not assembled with other ferritin molecules. A “ferritin multimer” comprises multiple associated ferritin monomers. A “ferritin protein” includes monomeric ferritin and multimeric ferritin.

As used herein, “ferritin particle,” refers to ferritin that has self-assembled into a globular form. Ferritin particles are sometimes referred to as “ferritin nanoparticles” or simply “nanoparticles”. In some embodiments, a ferritin particle comprises 24 ferritin monomers (or, where applicable, 24 total heavy and light chains).

“Hybrid ferritin,” as used herein, refers to ferritin comprising H. pylori ferritin with an amino terminal extension of bullfrog ferritin. An exemplary sequence used as an amino terminal extension of bullfrog ferritin appears as SEQ ID NO: 217. In hybrid ferritin, the amino terminal extension of bullfrog ferritin can be fused to H. pylori ferritin such that immune-stimulatory moiety attachment sites are distributed evenly on the ferritin particle surface. “Bullfrog linker” as used herein is a linker comprising the sequence of SEQ ID NO: 217. Hybrid ferritin is also sometimes referred to as “bfpFerr” or “bfp ferritin.” Any of the constructs comprising a bullfrog sequence can be provided without the bullfrog sequence, such as, for example, without a linker or with an alternative linker. Exemplary bullfrog linker sequences are provided in Table 1. Where Table 1 shows a bullfrog linker, the same construct may be made without a linker or with an alternative linker.

“N-glycan,” as used herein, refers to a saccharide chain attached to a protein at the amide nitrogen of an N (asparagine) residue of the protein. As such, an N-glycan is formed by the process of N-glycosylation. This glycan may be a polysaccharide.

“Glycosylation,” as used herein, refers to the addition of a saccharide unit to a protein.

“Immune response,” as used herein, refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. As used herein, a “protective immune response” refers to an immune response that protects a subject from infection (e.g., prevents infection or prevents the development of disease associated with infection). Methods of measuring immune responses are well known in the art and include, for example, by measuring proliferation and/or activity of lymphocytes (such as B or T cells), secretion of cytokines or chemokines, inflammation, antibody production and the like. An “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens include antigenic ferritin proteins comprising ferritin (e.g., comprising one or more mutations) and a non-ferritin polypeptide (e.g., RSV polypeptide) as described herein.

An “immune-stimulatory moiety,” as used herein, refers to a moiety that is covalently attached to a ferritin or antigenic ferritin polypeptide and that can activate a component of the immune system (either alone or when attached to ferritin or antigenic ferritin polypeptide). Exemplary immune-stimulatory moieties include agonists of toll-like receptors (TLRs), e.g., TLR 4, 7, 8, or 9. In some embodiments, an immune-stimulatory moiety is an adjuvant.

“Adjuvant,” as used herein, refers to a substance or vehicle that non-specifically enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules. An adjuvant may be administered as a separate molecule in a composition or covalently bound (conjugated) to ferritin or an antigenic ferritin polypeptide.

An “antigenic RSV polypeptide” is used herein to refer to a polypeptide comprising all or part of an RSV amino acid sequence of sufficient length that the molecule is antigenic with respect to RSV. Antigenicity may be a feature of the RSV sequence as part of a construct further comprising a heterologous sequence, such as a ferritin and/or immune-stimulatory moiety. That is, if an RSV sequence is part of a construct further comprising a heterologous sequence, then it is sufficient that the construct can serve as an antigen that generates anti-RSV antibodies, regardless of whether the RSV sequence without the heterologous sequence could do so.

“Antigenic ferritin polypeptide” and “antigenic ferritin protein” are used interchangeably herein to refer to a polypeptide comprising a ferritin and a non-ferritin polypeptide (e.g., an RSV polypeptide) of sufficient length that the molecule is antigenic with respect to the non-ferritin polypeptide. The antigenic ferritin polypeptide may further comprise an immune-stimulatory moiety. Antigenicity may be a feature of the non-ferritin sequence as part of the larger construct. That is, it is sufficient that the construct can serve as an antigen against the non-ferritin polypeptide, regardless of whether the non-ferritin polypeptide without the ferritin (and immune-stimulatory moiety if applicable) could do so. In some embodiments, the non-ferritin polypeptide is an RSV polypeptide, in which case the antigenic ferritin polypeptide is also an “antigenic RSV polypeptide.” To be clear, however, an antigenic RSV polypeptide does not need to comprise ferritin. “Antigenic polypeptide” is used herein to refer to a polypeptide which is either or both of an antigenic ferritin polypeptide and an antigenic RSV polypeptide.

“Self-adjuvanting,” as used herein, refers to a composition or polypeptide comprising a ferritin and an immune-stimulatory moiety directly conjugated to the ferritin so that the ferritin and immune-stimulatory moiety are in the same molecular entity. An antigenic ferritin polypeptide comprising a non-ferritin polypeptide may be conjugated to an immune-stimulatory moiety to generate a self-adjuvanting polypeptide.

A “surface-exposed” amino acid, as used herein, refers to an amino acid residue in a protein (e.g., a ferritin) with a side chain that can be contacted by solvent molecules when the protein is in its native three-dimensional conformation after multimerization, if applicable. Thus, for example, in the case of ferritin that forms a 24-mer, a surface-exposed amino acid residue is one whose side chain can be contacted by solvent when the ferritin is assembled as a 24-mer, e.g., as a ferritin multimer or ferritin particle.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, terms “individual” or “patient” are used and are intended to be interchangeable with “subject”.

As used herein, the term “vaccination” or “vaccinate” refers to the administration of a composition intended to generate an immune response, for example to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

The disclosure describes nucleic acid sequences and amino acid sequences having a certain degree of identity to a given nucleic acid sequence or amino acid sequence, respectively (a references sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes the ability to act as a cytokine, in particular when administered to a subject. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### B. Antigenic RSV Polypeptides Comprising an RSV F Polypeptide Comprising One or More Asparagines at Certain Positions

Provided herein are antigenic RSV polypeptides comprising an RSV F polypeptide. The RSV F polypeptide may comprise the whole sequence of RSV F or a portion of RSV F. In some embodiments, an epitope of the RSV polypeptide that is shared between pre-fusion RSV F and post-fusion RSV F is blocked. Blocking an epitope reduces or eliminates the generation of antibodies against the epitope when the antigenic RSV polypeptide is administered to a subject. This can increase the proportion of antibodies that target an epitope specific to a particular conformation of F, such as the pre-fusion conformation. Because F has the pre-fusion conformation in viruses that have not yet entered cells, an increased proportion of antibodies that target pre-fusion F can provide a greater degree of neutralization (e.g., expressed as a neutralizing to binding ratio, as described herein). Blocking can be achieved by engineering a bulky moiety such as an N-glycan in the vicinity of the shared epitope. For example, an N-glycosylation site not present in wild-type F can be added, e.g., by mutating an appropriate residue to asparagine. In some embodiments, the blocked epitope is an epitope of antigenic site 1 of RSV F. In some embodiments, two or more epitopes shared between pre-fusion RSV F and post-fusion RSV F are blocked. In some embodiments, two or more epitopes of antigenic site 1 of RSV F are blocked. In some embodiments, one or more, or all, epitopes that topologically overlap with the blocked epitope are also blocked, optionally wherein the blocked epitope is an epitope of antigenic site 1 of RSV F.

In some embodiments, the RSV F polypeptide comprises an asparagine corresponding to position 328, 348, or 507 of SEQ ID NO: 26. In some embodiments, the polypeptide comprises asparagines that correspond to at least two of positions 328, 348, or 507 of SEQ ID NO: 26. In some embodiments, the polypeptide comprises asparagines that correspond to positions 328, 348, or 507 of SEQ ID NO: 26. As described in the examples, it has been found that such asparagines can function as glycosylation sites. Furthermore, without wishing to be bound by any particular theory, glycans at these sites may inhibit development of antibodies to nearby epitopes, which include epitopes common to pre- and post-fusion RSV F protein, when the polypeptide is administered to a subject. In some embodiments, glycosylation of the asparagine corresponding to position 328, 348, or 507 of SEQ ID NO: 26 blocks at least one epitope shared between pre-fusion RSV F and post-fusion RSV F, such as an epitope of antigenic site 1. Inhibiting the development of antibodies to epitopes common to pre- and post-fusion RSV F protein can be beneficial because it can direct antibody development against epitopes specific to pre-fusion RSV F protein, such as the site 0 epitope, which may have more effective neutralizing activity than antibodies to other RSV F epitopes. The site 0 epitope involves amino acid residues 62-69 and 196-209 of SEQ ID NO: 26. Accordingly, in some embodiments, the RSV F polypeptide comprises amino acid residues 62-69 and 196-209 of SEQ ID NO: 26.

It should be noted that constructs described herein may have deletions or substitutions of different length relative to wild type RSV F. For example, in the construct of SEQ ID NO: 23 and others, positions 98-144 of the wild-type sequence (SEQ ID NO: 26) are replaced with GSGNVGL (positions 98-104 of SEQ ID NO: 23; also SEQ ID NO: 31), resulting in a net removal of 40 amino acids, such that positions 328, 348, or 507 of SEQ ID NO: 26 correspond to positions 288, 308, and 467 of SEQ ID NO: 23. In general, positions in constructs described herein can be mapped onto the wild-type sequence of SEQ ID NO: 26 by pairwise alignment, e.g., using the Needleman-Wunsch algorithm with standard parameters (EBLOSUM62 matrix, Gap penalty 10, gap extension penalty 0.5). See also the discussion of structural alignment provided herein as an alternative approach for identifying corresponding positions.

In some embodiments, the RSV F polypeptide comprises mutations that add glycans to block epitopes on the pre-fusion antigen that are structurally similar to those on the surface of the post-fusion RSV F. In some embodiments, glycans are added to specifically block epitopes that may be present in the post-fusion conformation of RSV F. In some embodiments, glycans are added that block epitopes that may be present in the post-fusion confirmation of RSV F but do not affect one or more epitopes present on the pre-fusion confirmation of RSV F, such as the site 0 epitope.

In some embodiments, the glycans added at the one or more glycosylation sites discussed above increase secretion in expression systems, such as mammalian cells, compared to other constructs.

In some embodiments, the RSV F polypeptide comprises a sequence having at least 85%, 90%, 95%, 97%, 98%, 99%, or 99.5% identity to amino acids 1-478 of SEQ ID NO: 17. In some embodiments, the RSV F polypeptide comprises a sequence having at least 85%, 90%, 95%, 97%, 98%, 99%, or 99.5% identity to the sequence of SEQ ID NO: 17. In some embodiments, the RSV F polypeptide comprises amino acids 1-478 of SEQ ID NO: 17. In some embodiments, the RSV F polypeptide comprises the sequence of SEQ ID NO: 17.

In some embodiments, the RSV F polypeptide comprises a sequence having at least 85%, 90%, 95%, 97%, 98%, 99%, or 99.5% identity to amino acids 1-478 of SEQ ID NO: 23. In some embodiments, the RSV F polypeptide comprises a sequence having at least 85%, 90%, 95%, 97%, 98%, 99%, or 99.5% identity to the sequence of SEQ ID NO: 23. In some embodiments, the RSV F polypeptide comprises amino acids 1-478 of SEQ ID NO: 23. In some embodiments, the RSV F polypeptide comprises the sequence of SEQ ID NO: 23.

In some embodiments, the RSV F polypeptide comprises the DS-CAV1 sequence (as described, for example, in McLellan, J. S., et al., Science 342(6158):592-598 (2013)) (SEQ ID NO: 25) in which further modifications are made including at least one, two, or three of the asparagines described above.

In some embodiments, the polypeptide further comprises a ferritin protein. The ferritin protein can further comprise any of the features described below in the section concerning ferritin, or a combination thereof.

The RSV F polypeptide can further comprise any of the additional features set forth in the following discussion, or any feasible combination of such features.

### Single Chain Constructs

In some embodiments, the RSV polypeptide is a single chain construct, e.g., an RSV polypeptide that lacks furin cleavage sites. In some embodiments, an RSV F lacks one or more furin cleavage sites. Constructs that lack furin cleavage sites are expressed as single polypeptides that are not cleaved into the biological F1/F2 fragments of the native F protein.

### Amino Acid Substitutions

In some embodiments, an RSV F comprises a single amino acid substitution relative to a wild-type sequence. In some embodiments, an RSV F comprises more than one single amino acid substitution, e.g., 2, 3, 4, 5, 6, 7, 8, 9, 10, 11, 12, 13, 14, or 15 substitutions relative to a wild-type sequence. An exemplary wild-type sequence is SEQ ID NO: 26.

In some embodiments, an amino acid substitution or pair of amino acid substitutions are inter-protomer stabilizing substitution(s). Exemplary substitutions that can be inter-protomer stabilizing are V207L; N228F; I217V and E218F; 1221L and E222M; or Q224A and Q225L, using the position numbering of SEQ ID NO: 26.

In some embodiments, an amino acid substitution or pair of amino acid substitutions are intra-protomer stabilizing. Exemplary substitutions that can be intra-protomer stabilizing are V220I; and A74L and Q81L, using the position numbering of SEQ ID NO: 26.

In some embodiments, an amino acid substitution is helix stabilizing, i.e., predicted to stabilize the helical domain of RSV F. Stabilization of the helical domain can contribute to the stability of the site 0 epitope and of the pre-fusion conformation of RSV F generally. Exemplary substitutions that can be helix stabilizing are N216P or I217P, using the position numbering of SEQ ID NO: 26.

In some embodiments, an amino acid substitution is helix capping. In some embodiments, an amino acid substitution is helix PRO capping. Helix capping is based on the biophysical observation that, while a proline residue mutation place in an alpha helix may disrupt the helix formation, a proline at the N-terminus of a helical region may help induce helical formation by stabilizing the PHI/PSI bond angles. Exemplary substitutions that can be helix capping are N216P or I217P, using the position numbering of SEQ ID NO: 26

In some embodiments, an amino acid substitution replaces a disulfide mutation of DS-CAV1. In some embodiments, the engineered disulfide of DS-CAV1 is reverted to wild-type (C69S and/or C212S mutations of DS-CAV1, using the position numbering of SEQ ID NO: 26. In some embodiments, one or more C residue of DS-CAV1 is replaced with a S residue to eliminate a disulfide bond. In some embodiments, C69S or C212S substitution using the position numbering of SEQ ID NO: 26 eliminates a disulfide bond. In some embodiments, an RSV F polypeptide comprises both C69S and C212S using the position numbering of SEQ ID NO: 26. In some embodiments, replacing such cysteines and thereby eliminating a disulfide bond blocks reduction (i.e. acceptance of electrons from a reducing agent) of the RSV F polypeptide. In some embodiments, an I217P substitution using the position numbering of SEQ ID NO: 26 is comprised in an antigen instead of substitution at C69 and/or C212. Position 217 in SEQ ID NO: 26 corresponds to position 177 in SEQ ID NO: 23.

In some embodiments, an amino acid substitution prevents proteolysis by trypsin or trypsin-like proteases. In some embodiments, the amino acid substitution that prevents such proteolysis is in the heptad repeat region B (HRB) region of RSV F. Appearance of fragments consistent with proteolysis of an RSV F-ferritin construct that comprised a wild-type HRB region suggested a lysine or arginine in this region was being targeted for proteolysis. An amino acid substitution to remove a K or R residue may be termed a knockout (KO). In some embodiments, a K or R is substituted for L or Q. In some embodiments, a K is substituted for L or Q. In some embodiments, the RSV F polypeptide comprises K498L and/or K508Q, using the position numbering of SEQ ID NO: 26. The corresponding positions in SEQ ID NO: 23 are 458 and 468, respectively. In some embodiments, the RSV F polypeptide comprises both K498L and K508Q.

In some embodiments, an amino acid substitution adds glycans. In some embodiments, an amino acid substitution increases glycosylation by adding glycans to RSV F polypeptides. Substitutions to add glycans may also be referred to as engineered glycosylation, as compared to native glycosylation (without additional glycans).

In some embodiments, the amino acid substitution to add glycans was substitution with an N. In some embodiments, amino acid substitution with an N allows N-linked glycosylation. In some embodiments, substitution with an N is accompanied by substitution with a T or S at the second amino acid position C-terminal to the N, which forms an NxT/S glycosylation motif. In some embodiments, the N is surface-exposed. As shown in the examples below, mutations that increased glycosylation could provide increased expression of a polypeptide comprising an RSV F polypeptide.

### Changes to the Properties of the RSV F Polypeptide Based on Modifications

Modifications to the amino sequence of RSV F can change the properties of an RSV F polypeptide. A property of an RSV F polypeptide can include any structural or functional characteristic of the RSV F polypeptide.

In some embodiments, a single modification to the amino acid sequence changes multiple properties of the RSV F polypeptide. In some embodiments, an RSV F polypeptide can comprise multiple modifications that change different properties of an RSV F polypeptide. In some embodiments, multiple modifications produce a greater change in the properties of an RSV F polypeptide.

In some embodiments, multiple modifications can have an additive effect on a particular property. For example, two amino acid substitutions to add glycans can produce a greater increase in glycosylation of the RSV F polypeptide compared to either single amino acid substitution.

In some embodiments, multiple modifications affect different properties of an RSV F polypeptide. For example, one or more amino acid substitutions to increase glycosylation can be made together with one or more amino acid substitutions to block reduction.

In some embodiments, modifications to an RSV F polypeptide stabilize the pre-fusion confirmation.

In some embodiments, modifications stabilize the site 0 epitope (also known as antigenic site 0) of pre-fusion RSV F, as described, for example, in McLellan et al., Science 340(6136):1113-1117 (2013). In some embodiments, a modification that stabilizes the site 0 epitope is inter-protomer stabilizing. In some embodiments, a modification that stabilizes the site 0 epitope stabilizes pre-fusion F, as measured by Site 0 and Site V binding as measured by binding to antibodies D25 or AM14, respectively.

In some embodiments, modifications increase expression of RSV F in expression systems. In some embodiments, modifications increase secretion of RSV F in expression systems. In some embodiments, modifications increase stability of the recombinant RSV F after expression. This change can be in any type of expression system, such as bacterial, fungal, insect, or mammalian.

In some embodiments, amino acid substitutions that introduce a proline increase expression compared to other constructs. In some embodiments, amino acid substitutions that add glycans increase expression compared to other constructs. In some embodiments, amino acid substitutions that substitute K or R for other amino acids increase expression compared to other constructs. An observable increase in expression can result from any mechanism that increases the yield of a fermentation run or other production process, including relative inhibition of protease cleavage or degradation and/or increase in stability in the host cell or in the extracellular milieu. In some embodiments, amino acid substitutions that substitute one or more K residues in the HRB region of RSV F for other amino acids increase expression compared to other constructs.

In some embodiments, amino acid substitutions that substitute K for other amino acids increase stability of RSV F polypeptides. In some embodiments, amino acid substitutions that substitute one or more K residues in the HRB region of RSV F for other amino acids increase stability of RSV F polypeptides. In some embodiments, this increased stability is due to a reduction in protease cleavage.

In some embodiments, an RSV F comprises mutation(s) that remove a disulfide, e.g., to prevent conjugation after reduction. In some embodiments, the I217P substitution blocks reduction of the RSV F polypeptide. In some embodiments, amino acid substitutions that substitute K for other amino acids block reduction of the RSV F polypeptide in the presence of a reducing agent.

In some embodiments, single chain constructs increase expression compared to other constructs.

In some embodiments, the RSV F polypeptide comprises the DS-CAV1 sequence (SEQ ID NO: 25) (as described in McLellan, J. S., et al., Science 342(6158):592-598 (2013)). In some embodiments, the RSV F polypeptide comprises the sequence of DS-CAV1 in which further modifications are made, e.g., including at least one, two, or three of the asparagines described above.

### C. RSV G Polypeptides

As used herein, an RSV G polypeptide may comprise the whole sequence of RSV G or a portion of RSV G. An RSV G polypeptide may comprise modifications compared to a wildtype sequence. In some embodiments, the RSV G polypeptide is an RSV G modified as compared to wild-type RSV G (SEQ ID NO: 27).

In some embodiments, these modifications are changes to the amino acid of the RSV G polypeptide as compared to wild-type RSV G.

In some embodiments, the RSV G polypeptide comprises all or part of the ectodomain of RSV G (SEQ ID NO: 28 or positions corresponding thereto). In some embodiments, the RSV G polypeptide comprises all or part of the Gcc region (amino acids 151-193 of RSV G (SEQ ID NO: 27)). In some embodiments, the RSV G polypeptide comprises a CX3C motif. In some embodiments, the RSV G polypeptide binds to the CX3CR1 receptor. The Gcc region is both conserved and immunogenic, and thus can be used to elicit antibodies with broad activity against RSV strains. In some embodiments, an RSV Gcc strain A is provided as shown in SEQ ID NO: 32. In some embodiments, an RSV Gcc strain B is provided as shown in SEQ ID NO: 33.

In some embodiments, the RSV G polypeptide is not glycosylated. For example, an RSV G polypeptide can lack NXS/TX glycosylation sites, either due to truncation or mutation of N or S/T residues (e.g., to Q or A, respectively), or a combination thereof.

In some embodiments, the RSV G polypeptide is part of an antigenic ferritin polypeptide. For example, the RSV G polypeptide can be conjugated to a ferritin as described herein, such as via a surface-exposed cysteine on the ferritin. In some embodiments, this ferritin nanoparticle is a fusion protein also comprising an RSV F polypeptide, such as any of the polypeptides comprising an RSV F polypeptide and a ferritin protein described above.

### D. Antigenic RSV Polypeptides Comprising Ferritin

Also provided herein is an antigenic RSV polypeptide comprising a ferritin and an RSV polypeptide. The RSV polypeptide can be an RSV F polypeptide, such as any of the RSV F polypeptides described herein. The RSV F polypeptide may comprise the whole sequence of RSV F or a portion of RSV F. The RSV F polypeptide may comprise one or more modification (e.g., amino acid substitution) compared to a wildtype sequence. The RSV polypeptide can be an RSV G polypeptide, such as any of the RSV G polypeptides described herein.

In some embodiments, the ferritin in the polypeptide is a wild-type ferritin. In some embodiments, the ferritin is bacterial, insect, fungal, bird, or mammalian. In some embodiments, the ferritin is human. In some embodiments, the ferritin is bacterial.

In some embodiments, the ferritin is a light chain and/or heavy chain ferritin. In some embodiments, the ferritin is an insect ferritin, such as Trichoplusia ni heavy chain ferritin (SEQ ID NO: 211) or Trichoplusia ni light chain ferritin (SEQ ID NO: 212). In some embodiments, the ferritin is a human ferritin, such as human heavy chain ferritin (SEQ ID NO: 214 or FTH1, GENE ID No: 2495) or human light chain ferritin (SEQ ID NO: 215 or FTL, GENE ID No: 2512). In some embodiments, a ferritin nanoparticle comprises 24 total subunits of heavy chain ferritin and light chain ferritin, such as in human or Trichoplusia ni ferritin nanoparticles. T. ni ferritin nanoparticles can comprise 12 subunits of heavy chain ferritin and 12 subunits of light chain ferritin.

In some embodiments, an antigenic RSV polypeptide comprises a light chain ferritin and an RSV polypeptide. In some embodiments, an antigenic RSV polypeptide comprises a heavy chain ferritin and an RSV polypeptide. In some embodiments, an antigenic RSV polypeptide comprising a light chain ferritin and an RSV polypeptide can assemble with a heavy chain ferritin that is not linked to an RSV polypeptide. In some embodiments, an antigenic RSV polypeptide comprising a heavy chain ferritin and an RSV polypeptide can assemble with a light chain ferritin that is not linked to an RSV polypeptide. A ferritin not linked to an RSV polypeptide (or, more generally, a non-ferritin polypeptide) may be referred as a “naked ferritin.”

In some embodiments, an antigenic polypeptide comprising a heavy chain ferritin and a polypeptide can assemble with an antigenic polypeptide comprising a light chain ferritin and an RSV polypeptide to allow presentation of two of the same or different non-ferritin polypeptides on a single ferritin nanoparticle. In some embodiments, the two different non-ferritin polypeptides are RSV polypeptides. In some embodiments, the two different non-ferritin polypeptides are encoded by RSV and a different infectious agent. In some embodiments, the different non-ferritin polypeptide from a different infectious agent is from a virus or bacterium.

In some embodiments, an antigenic polypeptide comprising a heavy chain ferritin and a non-ferritin polypeptide can assemble with a polypeptide comprising a light chain ferritin and a non-ferritin polypeptide to produce a bivalent composition, wherein one or both of the non-ferritin polypeptides are RSV polypeptides, such as RSV F or G polypeptides, e.g., an RSV F or G polypeptide described herein.

In some embodiments, the ferritin is H. pylori ferritin (see SEQ ID NO: 208 or 209 for an exemplary H. pylori ferritin sequence), optionally with one or more mutations such as those described herein. In some embodiments, the lower sequence homology between H. pylori ferritin (or other bacterial ferritins) and human ferritin may decrease the potential for autoimmunity when used as a vaccine platform (see Kanekiyo et al., Cell 162, 1090-1100 (2015)).

In some embodiments, the ferritin is Pyrococcus furiosus ferritin (NCBI seq WP_011011871.1) with one or more mutations described herein.

In some embodiments, the ferritin comprises a sequence having greater than 70%, greater than 75%, greater than 80%, greater than 85%, greater than 90%, greater than 95%, greater than 97%, greater than 98%, or greater than 99% identity to a wild-type ferritin.

In some embodiments, a nanoparticle is provided comprising an antigenic RSV polypeptide as disclosed herein comprising an RSV polypeptide and a ferritin.

In some embodiments, a different protein capable of forming a nanoparticle is substituted for ferritin. In some embodiments, this protein is lumazine synthase (see Ra et al., Clin Exp Vaccine Res 3:227-234 (2014)). In some embodiments, this protein is lumazine synthase serotype 1, 2, 3, 4, 5, 6, or 7. Exemplary lumazine synthase sequences are provided as SEQ ID NO: 216 and 219. In some embodiments, the lumazine synthase comprises a sequence with 80%, 85%, 90%, 95%, 98%, or 99% identity to SEQ ID NO: 216 or 219.

### 1. Cysteine for Conjugation

In some embodiments, ferritin is mutated to provide a chemical handle for conjugation of an immune-stimulatory moiety and/or RSV polypeptide. This can be achieved with a mutation replacing a surface-exposed non-cysteine amino acid with a cysteine. For the avoidance of doubt, language such as “replacing a surface-exposed amino acid with a cysteine” necessarily implies that the surface-exposed amino acid in the wild-type or pre-mutation sequence is not cysteine. Another approach for providing a chemical handle for conjugation of an immune-stimulatory moiety or RSV polypeptide is to include a segment of amino acids, such as a linker, N- or C-terminal to the ferritin, wherein the segment of amino acids comprises a cysteine. In some embodiments, this cysteine (whether replacing a surface-exposed amino acid or in an N- or C-terminal linker) is unpaired, which means that it does not have an appropriate partner cysteine to form a disulfide bond. In some embodiments, this cysteine does not change the secondary structure of ferritin. In some embodiments, this cysteine does not change the tertiary structure of ferritin.

In some embodiments, this cysteine can be used to conjugate agents, such as immune-stimulatory moieties, to ferritin. In some embodiments, this cysteine provides a free thiol group that is reactive. In some embodiments, agents conjugated to this cysteine on ferritin are exposed on the surface of an assembled ferritin particle. In some embodiments, this cysteine can interact with molecules and cells of the subject after administration while the ferritin particle is assembled.

In some embodiments, the presence of this cysteine allows conjugation of one or more immune-stimulatory moieties, e.g., adjuvants. In some embodiments, conjugation of the immune-stimulatory moiety would not occur in the absence of this cysteine.

In some embodiments, the non-cysteine amino acid that is replaced with a cysteine is selected from E12, S72, A75, K79, S100, and S111 of H. pylori ferritin. Thus, in some embodiments, the surface-exposed amino acid that is replaced in favor of cysteine is an amino acid residue that corresponds to E12, S26, S72, A75, K79, S100, or S111 of H. pylori ferritin. Analogous amino acids can be found in non-H. pylori ferritin by pair-wise or structural alignment. In some embodiments, the non-cysteine amino acid that is replaced with a cysteine can be selected from an amino acid that corresponds to S3, S19, S33, 182, A86, A102, and A120 of human light chain ferritin. In some embodiments, the surface-exposed amino acid to be replaced with a cysteine is selected based on the understanding that if the native amino acid were replaced with cysteine, it would be reactive in an assembled ferritin multimer or particle and/or that this cysteine does not disrupt the stability of the ferritin multimer or particle and/or that this cysteine does not lead to reduction in expression levels of ferritin.

In some embodiments, the ferritin comprises an E12C mutation. In some embodiments, the E12C residue can be used to conjugate agents (e.g., immune-stimulatory moieties and/or RSV polypeptides) to ferritin. In some embodiments, the E12C residue provides a free thiol group that is reactive. In some embodiments, agents conjugated to the E12C residue on ferritin monomers are expressed on the surface on an assembled ferritin multimer or particle. In some embodiments, twenty-four E12C residues (one from each monomer) are present on the surface of a ferritin multimer or particle.

In some embodiments, the ferritin comprises an S26C mutation. In some embodiments, the S26C residue can be used to conjugate agents (e.g., immune-stimulatory moieties and/or RSV polypeptides) to ferritin. In some embodiments, the S26C residue provides a free thiol group that is reactive. In some embodiments, agents conjugated to the S26C residue on ferritin monomers are expressed on the surface on an assembled ferritin multimer or particle. In some embodiments, twenty-four S26C residues (one from each monomer) are present on the surface of a ferritin multimer or particle.

In some embodiments, the ferritin comprises an S72C mutation. In some embodiments, the S72C residue can be used to conjugate agents (e.g., immune-stimulatory moieties and/or RSV polypeptides) to ferritin. In some embodiments, the S72C residue provides a free thiol group that is reactive. In some embodiments, agents conjugated to the S72C residue on ferritin monomers are expressed on the surface on an assembled ferritin multimer or particle. In some embodiments, twenty-four S72C residues (one from each monomer) are present on the surface of a ferritin multimer or particle.

In some embodiments, the ferritin comprises an A75C mutation. In some embodiments, the A75C residue can be used to conjugate agents (e.g., immune-stimulatory moieties and/or RSV polypeptides) to ferritin. In some embodiments, the A75C residue provides a free thiol group that is reactive. In some embodiments, agents conjugated to the A75C residue on ferritin monomers are expressed on the surface on an assembled ferritin multimer or particle. In some embodiments, twenty-four A75C residues (one from each monomer) are present on the surface of a ferritin multimer or particle.

In some embodiments, the ferritin comprises an K79C mutation. In some embodiments, the K79C residue can be used to conjugate agents (e.g., immune-stimulatory moieties and/or RSV polypeptides) to ferritin. In some embodiments, the K79C residue provides a free thiol group that is reactive. In some embodiments, agents conjugated to the K79C residue on ferritin monomers are expressed on the surface on an assembled ferritin multimer or particle. In some embodiments, twenty-four K79C residues (one from each monomer) are present on the surface of a ferritin multimer or particle.

In some embodiments, the ferritin comprises an S100C mutation. In some embodiments, the S100C residue can be used to conjugate agents (e.g., immune-stimulatory moieties and/or RSV polypeptides) to ferritin. In some embodiments, the S100C residue provides a free thiol group that is reactive. In some embodiments, agents conjugated to the S100C residue on ferritin monomers are expressed on the surface on an assembled ferritin multimer or particle. In some embodiments, twenty-four S100C residues (one from each monomer) are present on the surface of a ferritin multimer or particle.

In some embodiments, the ferritin comprises an S111C mutation. In some embodiments, the S111C residue can be used to conjugate agents (e.g., immune-stimulatory moieties and/or RSV polypeptides) to ferritin. In some embodiments, the S111C residue provides a free thiol group that is reactive. In some embodiments, agents conjugated to the S111C residue on ferritin monomers are expressed on the surface on an assembled ferritin multimer or particle. In some embodiments, twenty-four S111C residues (one from each monomer) are present on the surface of a ferritin multimer or particle.

### 2. Removal of Internal Cysteine

In some embodiments, the ferritin comprises a mutation replacing an internal cysteine with a non-cysteine amino acid. Removal of a native internal cysteine residue can ensure that there is only one unpaired cysteine per ferritin monomer and avoid undesired reactions such as disulfide formation and may result in a more stable and efficient result (e.g., adjuvant presentation). In some embodiments, C31 of H. pylori ferritin is replaced with a non-cysteine amino acid. In some embodiments, C31 of H. pylori ferritin is replaced with a serine (C31S), although any non-cysteine residue may be used, e.g., alanine, glycine, threonine, or asparagine. Analogous amino acids can be found in non-H. pylori ferritin by pair-wise or structural alignment. Thus, in some embodiments, the internal cysteine that is replaced in favor of non-cysteine is an amino acid residue that aligns with C31 of H. pylori ferritin. Exemplary ferritin sequences showing a C31S mutation are shown in SEQ ID NOS: 201-207. In some embodiments, when more than one internal cysteine is present in ferritin, two or more (e.g., each) internal cysteine is replaced with a non-cysteine amino acid, such as serine or an amino acid selected from serine, alanine, glycine, threonine, or asparagine.

### 3. Glycosylation

Human-compatible glycosylation can contribute to safety and efficacy in recombinant drug products. Regulatory approval may be contingent on demonstrating appropriate glycosylation as a critical quality attribute (see Zhang et al., Drug Discovery Today 21(5):740-765 (2016)). N-glycans can result from glycosylation of asparagine side chains and can differ in structure between humans and other organisms such as bacteria and yeast. Thus, it may be desirable to reduce or eliminate non-human glycosylation and/or N-glycan formation in ferritin according to the disclosure. In some embodiments, controlling glycosylation of ferritin improves the efficacy and/or safety of the composition, especially when used for human vaccination.

In some embodiments, ferritin is mutated to inhibit formation of an N-glycan. In some embodiments, a mutated ferritin has reduced glycosylation as compared to its corresponding wild type ferritin.

In some embodiments, the ferritin comprises a mutation replacing a surface-exposed asparagine with a non-asparagine amino acid. In some embodiments, the surface-exposed asparagine is N19 of H. pylori ferritin or a position that corresponds to position 31 of H. pylori ferritin as determined by pair-wise or structural alignment In some embodiments, mutating such an asparagine, e.g., N19 of H. pylori ferritin, decreases glycosylation of ferritin. In some embodiments, the mutation replaces the asparagine with a glutamine. In some embodiments, the ferritin is an H. pylori ferritin comprising an N19Q mutation. SEQ ID NOS: 201-207 are exemplary ferritin sequences comprising N19Q mutations.

A mammal exposed to a glycosylated protein produced in bacteria or yeast may generate an immune response to the glycosylated protein, because the pattern of glycosylation of a given protein in bacterial or yeast could be different from the pattern of glycosylation of the same protein in a mammal. Thus, some glycosylated therapeutic proteins may not be appropriate for production in bacteria or yeast.

In some embodiments, decreased glycosylation of ferritin by amino acid mutation facilitates protein production in bacteria or yeast. In some embodiments, decreased glycosylation of ferritin reduces the potential for adverse effects in mammals upon administration of mutated ferritin that is expressed in bacteria or yeast. In some embodiments, the reactogenicity in a human subject of a mutated ferritin produced in bacteria or yeast is lower because glycosylation is decreased. In some embodiments, the incidence of hypersensitivity responses in human subjects is lower following treatment with a mutated ferritin with reduced glycosylation compared to wildtype ferritin.

In some embodiments, degradation in a subject of a composition comprising a mutated ferritin with reduced glycosylation is slower compared with a composition comprising a wild-type ferritin, or a composition comprising a corresponding ferritin with wild-type glycosylation. In some embodiments, a composition comprising a mutated ferritin with reduced glycosylation has reduced clearance in a subject compared with a composition comprising a wild-type ferritin, or a composition comprising a corresponding ferritin with wild-type glycosylation. In some embodiments, a composition comprising a mutated ferritin with reduced glycosylation has a longer-serum half-life compared to wild-type ferritin, or a composition comprising a corresponding ferritin with wild-type glycosylation.

### 4. Combinations of Mutations

In some embodiments, a ferritin comprises more than one type of mutation described herein. In some embodiments, the ferritin comprises one or more mutations independently selected from: a mutation to decrease glycosylation, a mutation to remove an internal cysteine, and a mutation to generate a surface-exposed cysteine. In some embodiments, the ferritin comprises a mutation to decrease glycosylation, a mutation to remove an internal cysteine, and a mutation to generate a surface-exposed cysteine.

In some embodiments, the ferritin comprises an N19Q mutation, a C31S mutation, and a mutation to generate a surface-exposed cysteine. In some embodiments, the ferritin comprises an N19Q mutation, a C31S mutation, and an E12C mutation. In some embodiments, the ferritin comprises an N19Q mutation, a C31S mutation, and an S72C mutation. In some embodiments, the ferritin comprises an N19Q mutation, a C31S mutation, and an A75C mutation. In some embodiments, the ferritin comprises an N19Q mutation, a C31S mutation, and an K79C mutation. In some embodiments, the ferritin comprises an N19Q mutation, a C31S mutation, and an S100C mutation. In some embodiments, the ferritin comprises an N19Q mutation, a C31S mutation, and an S111C mutation. In some embodiments, the ferritin comprises mutations corresponding to any of the foregoing sets of mutations, wherein the corresponding mutations change an N to a Q, a C to an S, and a non-cysteine surface-exposed amino acid to a cysteine at positions determined by pair-wise alignment of the ferritin amino acid sequence to an H. pylori ferritin amino acid sequence (SEQ ID NO: 208 or 209).

Exemplary ferritins comprising more than one type of mutation are provided in SEQ ID NOS: 201-207.

### 5. Structural Alignment

As discussed herein, positions of mutations corresponding to those described with respect to a given polypeptide (e.g, H. pylori ferritin) can be identified by pairwise or structural alignment. Structural alignment is relevant to large protein families such as ferritin where the proteins share similar structures despite considerable sequence variation and many members of the family have been structurally characterized, and can also be used to identify corresponding positions in different versions of other polypeptides described herein, such as RSV polypeptides (e.g., RSV F or G). The protein databank (PDB) comprises 3D structures for many ferritins, including those listed below with their accession numbers.

2jd6, 2jd7—PfFR—Pyrococcus furiosus. 2jd8—PfFR+Zn. 3a68—soFR from gene SferH4—soybean. 3a9q—soFR from gene SferH4 (mutant). 3egm, 3bvf, 3bvi, 3bvk, 3bvl—HpFR—Heliobacter pylori. 5c6f—HpFR (mutant)+Fe. 1z4a, 1v1g—FR—Thermotoga maritime. 1s3q, 1sq3, 3kx9—FR—Archaeoglubus fulgidus. 1krq—FR—Campylobacter jejuni. 1eum—EcFR—Escherichia coli. 4reu—EcFR+Fe. 4xgs—EcFR (mutant)+Fe2O2. 4ztt—EcFR (mutant)+Fe2O+Fe2+Fe+O2. 1qgh—LiFR—Listeria innocua. 3qz3—VcFR—Vibrio cholerae. 3vnx—FR—Ulva pertusa. 4ism, 4isp, 4itt, 4itw, 4iwj, 4iwk, 4ixk, 3e6s—PnmFR—Pseudo-nitschia multiseries. 4zkh, 4zkw, 4zkx, 4z15, 4z16, 4zlw, 4zmc—PnmFR (mutant)+Fe. 1z6o—FR—Trichoplusia ni. 4cmy—FR+Fe—Chlorobaculum tepidum. Ferritin light chain (FTL). 11b3, 1h96—mFTL—mouse. 1rcc, 1rcd, 1rci—bFTL+tartrate+Mg. 1rce, 1reg—bFTL+tartrate+Mn. 3noz, 3np0, 3np2, 3o7r—hoFTL (mutant)—horse. 3o7s, 3u90—hoFTL. 4v1w—hoFTL—cryo EM. 3rav, 3rd0—hoFTL+barbiturate. Ferritin light+heavy chains: 5gn8—hFTH+Ca.

Structural alignment involves identifying corresponding residues across two (or more) polypeptide sequences by (i) modeling the structure of a first sequence using the known structure of the second sequence or (ii) comparing the structures of the first and second sequences where both are known, and identifying the residue in the first sequence most similarly positioned to a residue of interest in the second sequence. Corresponding residues are identified in some algorithms based on alpha-carbon distance minimization in the overlaid structures (e.g., what set of paired alpha carbons provides a minimized root-mean-square deviation for the alignment). When identifying positions in a non-H. pylori ferritin corresponding to positions described with respect to H. pylori ferritin, H. pylori ferritin can be the “second” sequence. Where a non-H. pylori ferritin of interest does not have an available known structure, but is more closely related to another non-H. pylori ferritin that does have a known structure than to H. pylori ferritin, it may be most effective to model the non-H. pylori ferritin of interest using the known structure of the closely related non-H. pylori ferritin, and then compare that model to the H. pylori ferritin structure to identify the desired corresponding residue in the ferritin of interest. There is an extensive literature on structural modeling and alignment; representative disclosures include U.S. Pat. Nos. 6,859,736; 8,738,343; and those cited in Aslam et al., Electronic Journal of Biotechnology 20 (2016) 9-13. For discussion of modeling a structure based on a known related structure or structures, see, e.g., Bordoli et al., Nature Protocols 4 (2009) 1-13, and references cited therein.

### 6. Immune-Stimulatory Moieties; Adjuvants; Conjugated RSV Polypeptides

In some embodiments, an RSV polypeptide and/or an immune-stimulatory moiety, such as an adjuvant, is attached to a surface-exposed amino acid. In some embodiments, the surface-exposed amino acid is a cysteine, e.g., resulting from a mutation discussed above. In some embodiments, the surface-exposed amino acid is a lysine, aspartate, or glutamate. Conjugation procedures using glutaraldehyde (for conjugation of a lysine with an amino-bearing linker or moiety) or a carbodiimide (e.g., 1-Cyclohexyl-3-(2-morpholin-4-yl-ethyl) carbodiimide or 1-Ethyl-3-(3-dimethyl-aminopropyl) carbodiimide (EDC; EDAC) for conjugating an aspartate or glutamate to an amino-bearing linker or moiety, or a lysine to a carboxyl-bearing linker or moiety) are described in, e.g., Chapter 4 of Holtzhauer, M., Basic Methods for the Biochemical Lab, Springer 2006, ISBN 978-3-540-32785-1, available from www.springer.com.

In some embodiments, an immune-stimulatory moiety, such as an adjuvant, is attached to a surface-exposed amino acid of ferritin. In some embodiments, more than one immune-stimulatory moiety, such as an adjuvant, is attached to a surface-exposed amino acid of ferritin. In some embodiments, twenty-four immune-stimulatory moieties are attached to a ferritin multimer or particle (e.g., one moiety for each monomer in the H. pylori ferritin particle). In some embodiments with multiple immune-stimulatory moieties attached to a ferritin nanoparticle, all of the immune-stimulatory moieties are identical. In some embodiments with multiple immune-stimulatory moieties attached to a ferritin nanoparticle, all of the immune-stimulatory moieties are not identical.

### a) Types of Immune-Stimulatory Moieties; Adjuvants

Any immune-stimulatory moiety that can be attached to a surface-exposed amino acid (e.g., cysteine) can be used in ferritins according to this disclosure. In some embodiments, the immune-stimulatory moiety is a B cell agonist.

In some embodiments, the immune-stimulatory moiety is not hydrophobic. In some embodiments, the immune-stimulatory moiety is hydrophilic. In some embodiments, the immune-stimulatory moiety is polar. In some embodiments, the immune-stimulatory moiety is capable of hydrogen bonding or ionic bonding, e.g., comprises a hydrogen bond donor, hydrogen bond acceptor, cationic moiety, or anionic moiety. A moiety is considered cationic or anionic if it would be ionized in aqueous solution at a physiologically relevant pH, such as pH 6, 7, 7.4, or 8.

In some embodiments, the immune-stimulatory moiety is an adjuvant. In some embodiments, the adjuvant comprises a pathogen associated molecular pattern (PAMP). In some embodiments, the adjuvant is a toll-like receptor (TLR) agonist or stimulator of interferon genes (STING) agonist. In some embodiments, the adjuvant activates TLR signaling in B and/or T cells. In some embodiments, the adjuvant regulates the adaptive immune response.

### (1) TLR2 Agonists

In some embodiments, the immune-stimulatory moiety is a TLR2 agonist. In some embodiments, the immune-stimulatory moiety stimulates TLR2 signaling. In some embodiments, the immune-stimulatory moiety is a synthetic small molecule ligand of TLR2. In some embodiments, the immune-stimulatory moiety is a synthetic small molecule agonist of TLR2 signaling.

In some embodiments, the TLR2 agonist is PAM2CSK4, FSL-1, or PAM3CSK4.

### (2) TLR7 8 Agonists

In some embodiments, the immune-stimulatory moiety is a TLR7 and/or TLR8 agonist (i.e., an agonist of at least one of TLR7 and TLR8). In some embodiments, the immune-stimulatory moiety stimulates TLR7 and/or TLR8 signaling. In some embodiments, the immune-stimulatory moiety is a synthetic small molecule ligand of TLR7 and/or TLR8. In some embodiments, the immune-stimulatory moiety is a synthetic small molecule agonist of TLR7 and/or TLR8 signaling.

In some embodiments, the TLR7 and/or TLR8 agonist is single-stranded (ssRNA). In some embodiments, the TLR7 and/or TLR8 agonist is an imidazoquinoline. In some embodiments, the TLR7 and/or TLR8 agonist is a nucleoside analog.

In some embodiments, the TLR7 and/or TLR8 agonist is an imidazoquinolinamine Toll-like receptor (TLR) agonist, such as 3M-012 (3M Pharmaceuticals). The structure of free 3M-012 is:

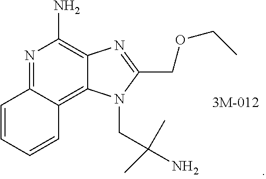 [SMILES: CCOCc1nc2c(N)nc3ccccc3c2n1CC(C)(C)N]

It is understood that an immune-stimulatory moiety such as 3M-012 or any moiety discussed herein can be conjugated to a ferritin by substituting an appropriate peripheral atom of the moiety (e.g., a hydrogen) with a bond to a ferritin described herein, e.g., at the sulfur of a surface-exposed cysteine or a linker attached to such a sulfur. Thus, when conjugated to a ferritin, the structure of the immune-stimulatory moiety will differ slightly from the structure of the free molecule.

In some embodiments the TLR7 and/or TLR8 agonist is SM 7/8a. The structure of free SM 7/8a is:

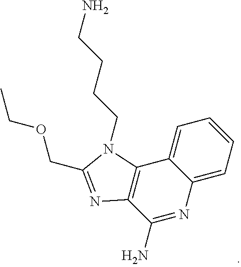 [SMILES: CCOCc1nc2c(N)nc3ccccc3c2n1CCCCN]

See, e.g., Nat Biotechnol. 2015 November; 33(11):1201-10. doi: 10.1038/nbt.3371.

### (3) TLR9 Agonists

In some embodiments, the immune-stimulatory moiety is a TLR9 agonist. In some embodiments, the immune-stimulatory moiety stimulates TLR9 signaling. In some embodiments, the immune-stimulatory moiety is a synthetic small molecule ligand of TLR9. In some embodiments, the immune-stimulatory moiety is a synthetic small molecule agonist of TLR9 signaling.

In some embodiments, the TLR9 agonist is a CpG oligodeoxynucleotide (ODN). In some embodiments, the TLR9 agonist is an unmethylated CpG ODN. In some embodiments, the CpG ODN comprises a partial or complete phosphorothioate (PS) backbone instead of the natural phosphodiester (PO) backbone found in ordinary DNA.

In some embodiments, the CpG ODN is a Class B ODN, which comprises one or more 6mer CpG motif comprising 5′ Purine (Pu)-Pyrimidine (Py)-C-G-Py-Pu 3′; has a fully phosphorothioated (i.e., PS-modified) backbone; and has a length of 18-28 nucleotides. In some embodiments, the CpG ODN comprises the sequence of SEQ ID NO: 210, optionally comprising phosphorothioate linkages in the backbone.

In some embodiments, the TLR9 agonist comprises an immune-stimulatory sequence (ISS). In some embodiments the TLR9 agonist is ISS-1018 (Dynavax) (SEQ ID NO: 210).

### (4) STING Agonists

In some embodiments, the immune-stimulatory moiety is a STING (Stimulator of Interferon Genes Protein, also known as Endoplasmic Reticulum IFN Stimulator) agonist. In some embodiments, the immune-stimulatory moiety stimulates STING signaling. In some embodiments, the immune-stimulatory moiety is a synthetic small molecule ligand of STING. In some embodiments, the immune-stimulatory moiety is a synthetic small molecule agonist of STING signaling.

In some embodiments the STING agonist is a cyclic dinucleotide (CDN). See, e.g., Danilchanka et al., Cell 154:962-970 (2013). Exemplary CDNs include cdA, cdG, cAMP-cGMP, and 2′-5′,3′-5′ cGAMP (see Danilchanka et al. for structures). STING agonists also include synthetic agonists such as DMXAA

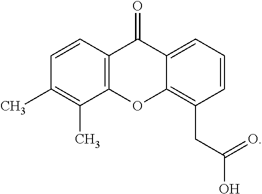 [SMILES: Cc1ccc2c(=O)c3cccc(CC(=O)O)c3oc2c1C]

### b) Conjugated RSV Polypeptides

In some embodiments, an RSV polypeptide is conjugated to a surface-exposed amino acid of ferritin. In some embodiments, the RSV polypeptide renders the ferritin protein antigenic. In some embodiments, the RSV polypeptide is antigenic alone, whereas in some embodiments, the RSV polypeptide is antigenic because of its association with ferritin. In some embodiments, the RSV polypeptide is any one of the RSV F or G polypeptides described herein.

### c) Conjugation

In some embodiments, a surface-exposed cysteine (e.g., resulting from a mutation described herein) or a cysteine in a peptide linker attached to ferritin (e.g., N-terminally to ferritin) is used to conjugate an immune-stimulatory moiety, such as an adjuvant, or an RSV polypeptide to a ferritin. In some embodiments, a linker is conjugated to such a cysteine, which linker can be subsequently conjugated to an immune-stimulatory moiety, such as an adjuvant, or an RSV polypeptide. In some embodiments, such a cysteine creates a chemical handle for conjugation reactions to attach an adjuvant, linker, or an RSV polypeptide. In some embodiments, bioconjugates are produced, wherein an immune-stimulatory moiety, such as an adjuvant, or an RSV polypeptide is linked to a ferritin after reduction of such a cysteine. In some embodiments, the cysteine is an unpaired surface-exposed cysteine, i.e., that lacks a partner cysteine in an appropriate position to form a disulfide bond. In some embodiments, the cysteine is an unpaired cysteine that comprises a free thiol side chain.

### (1) Types of Conjugation Chemistries

Any type chemistry can be used to conjugate the immune-stimulatory moiety, such as an adjuvant, or an RSV polypeptide to the ferritin, e.g., via reaction a surface-exposed amino acid such as cysteine or another amino acid such as Lys, Glu, or Asp.

In some embodiments, the conjugation is performed using click chemistry. As used herein, “click chemistry” refers to a reaction between a pair of functional groups that rapidly and selective react (i.e., “click”) with each other. In some embodiments, the click chemistry can be performed under mild, aqueous conditions. In some embodiments, a click chemistry reaction takes advantage of a cysteine on the surface of the ferritin, such as a cysteine resulting from mutation of a surface-exposed amino acid, to perform click chemistry using a functional group that can react with the cysteine.

A variety of reactions that fulfill the criteria for click chemistry are known in the field, and one skilled in the art could use any one of a number of published methodologies (see, e.g., Hein et al., Pharm Res 25(10):2216-2230 (2008)). A wide range of commercially available reagents for click chemistry could be used, such as those from Sigma Aldrich, Jena Bioscience, or Lumiprobe. In some embodiments, conjugation is performed using click chemistry as described in the Examples below.

In some embodiments, the click chemistry reaction occurs after reduction of the ferritin.

In some embodiments, the click chemistry may be a 1-step click reaction. In some embodiments, the click chemistry may be a 2-step click reaction.

In some embodiments, the reaction(s) comprises metal-free click chemistry. In some embodiments, the reaction(s) comprise thiol-maleimide and/or disulfide exchange.

### Metal-Free Click Chemistry

Metal-free click chemistry can be used for conjugation reactions to avoid potential oxidation of proteins. Metal-free click chemistry has been used to form antibody conjugates (see van Geel et al., Bioconjugate Chem. 2015, 26, 2233-2242).

In some embodiments, metal-free click chemistry is used in reactions to attach adjuvant to ferritin. In some embodiments, copper-free conjugation is used in reactions to attach adjuvant to ferritin. In some embodiments, the metal-free click chemistry uses bicyclo[6.1.0]nonyne (BCN). In some embodiments, the metal-free click chemistry uses dibenzoazacyclooctyne (DBCO). In some embodiments BCN or DBCO reacts with an azide group.

DBCO has high specificity for azide groups via a strain-promoted click reaction in the absence of a catalyst, resulting in high yield of a stable triazole. In some embodiments, DBCO reacts with azide in the absence of copper catalyst.

In some embodiments, metal-free click chemistry is used in a 1-step click reaction. In some embodiments, metal-free click chemistry is used in a 2-step click reaction.

### Thiol-Maleimide and Disulfide Exchange

Ferritins described herein can comprise a cysteine comprising a thiol, also known as a sulfhydryl, which is available for reaction with sulfhydryl-reactive chemical groups (or which can be made available through reduction). Thus, the cysteine allows chemoselective modification to add an immune-stimulatory moiety, such as an adjuvant, to the ferritin. Under basic conditions, the cysteine will be deprotonated to generate a thiolate nucleophile, which can react with soft electrophiles, such as maleimides and iodoacetamides. The reaction of the cysteine with a maleimide or iodoacetamide results in a carbon-sulfur bond.

In some embodiments, a sulfhydryl-reactive chemical group reacts with the surface-exposed cysteine or cysteine in the linker of the ferritin. In some embodiments, the sulfhydryl-reactive chemical group is a haloacetyl, maleimide, aziridine, acryloyl, arylating agent, vinylsulfone, pyridyl disulfide, or TNB-thiol.

In some embodiments, the sulfhydryl-reactive chemical group conjugates to the sulfhydryl of the cysteine by alkylation (i.e., formation of a thioether bond)). In some embodiments, the sulfhydryl-reactive chemical group conjugates to the sulfhydryl of the cysteine by disulfide exchange (i.e., formation of a disulfide bond).

In some embodiments, the reaction to conjugate an immune-stimulatory moiety, such as an adjuvant, to the ferritin is a thiol-maleimide reaction.

In some embodiments, the sulfhydryl-reactive chemical group is a maleimide. In some embodiments, reaction of a maleimide with the cysteine results in formation of a stable thioester linkage, e.g., that is not reversible. In some embodiments, the maleimide does not react with tyrosines, histidines, or methionines in the ferritin. In some embodiments, unreacted maleimides are quenched at the end of the reaction by adding a free thiol, e.g., in excess.

In some embodiments, the reaction to conjugate an immune-stimulatory moiety, such as an adjuvant, to the ferritin is a thiol-disulfide exchange, also known as a disulfide interchange. In some embodiments, the reaction involves formation of a mixed disulfide comprising a portion of the original disulfide. In some embodiments, the original disulfide is the cysteine introduced in the ferritin by mutation of a surface-exposed amino acid or addition of an N-terminal linker.

In some embodiments, the sulfhydryl-reactive chemical group is a pyridyl dithiol. In some embodiments, the sulfhydryl-reactive chemical group is a TNB-thiol group.

### (2) Conjugated Linkers

In some embodiments, an immune-stimulatory moiety, such as an adjuvant, or an RSV polypeptide is attached to the ferritin via a linker that is covalently bound to a surface-exposed amino acid such as a cysteine. In some embodiments, the linker comprises a polyethylene glycol, e.g., a PEG linker. In some embodiments, the polyethylene glycol (e.g., PEG) linker increases water solubility and ligation efficiency of the ferritin linked to the immune-stimulatory moiety, such as an adjuvant. The PEG linker is between 2 and 18 PEGs long, e.g., PEG4, PEG5, PEG6, PEG7, PEG8, PEG9, PEG10, PEG11, PEG12, PEG13, PEG14, PEG15, PEG16, PEG17, and PEG18.

In some embodiments, the linker comprises a maleimide. In some embodiments, the linker comprises the components of immune-stimulatory moiety (ISM)-linker-maleimide. In some embodiments, the ISM-linker-maleimide is conjugated to ferritin in a 1-step click chemistry reaction by reaction of the maleimide with a cysteine of the ferritin. In some embodiments, the ISM of the adjuvant-linker-maleimide is SM7/8a. In some embodiments, the linker of the ISM-linker-maleimide is PEG4. In some embodiments, the ISM-linker-maleimide is SM7/8a-PEG4-maleimide.

In some embodiments, a 2-step click chemistry protocol is used with a linker comprising a sulfhydryl-reactive chemical group at one end and an amine-reactive group at the other end. In such a 2-step click chemistry protocol, a sulfhydryl-reactive chemical group reacts with a cysteine of the ferritin, while the amine-reactive group reacts with a reagent attached to the ISM. In this way, the ISM is conjugated to the ferritin via a set of 2 click chemistry reagents.

In some embodiments of the 2-step click chemistry protocol, the sulfhydryl-reactive chemical group is maleimide. In some embodiments of the 2-step click chemistry protocol, the maleimide reacts with the cysteine introduced in the ferritin by mutation of a surface-exposed amino acid or addition of an N-terminal linker.

In some embodiments of the 2-step click chemistry protocol, the amine-reactive group is DBCO. In some embodiments of the 2-step click chemistry protocol, the DBCO reacts with an azide group attached to an ISM.

In some embodiments, a maleimide-linker-DBCO is used. In some embodiments, the maleimide-linker-DBCO is conjugated to ferritin after the ferritin is reduced. In some embodiments, the maleimide-linker-reagent is conjugated to ferritin by reaction of the maleimide with the cysteine of the ferritin in a first step. In some embodiments, the DBCO is used to link to an ISM attached to azide. In some embodiments, the ISM coupled to azide is ISS-1018. In some embodiments, the adjuvant coupled to azide is 3M-012 or CpG.

In some embodiments, a linker with a reactive group is added to the ISM. In some embodiments, the linker is a PEG4-azide linker or a PEG4-maleimide linker.

In some embodiments, a PEG4-azide linker is conjugated to 3M-012. An exemplary structure of 3M-012 conjugated to a PEG4-azide linker is:

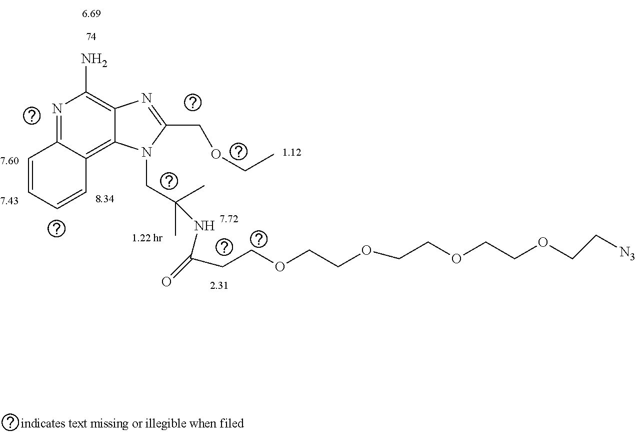 [SMILES: CCOCc1nc2c(N)nc3ccccc3c2n1CC(C)(C)NC(=O)CCOCCOCCOCCOCCN=[N+]=[N-]]

In some embodiments, a PEG4-azide linker is conjugated to SM7/8a. An exemplary structure of SM7/8a conjugated to a PEG4-azide linker is:

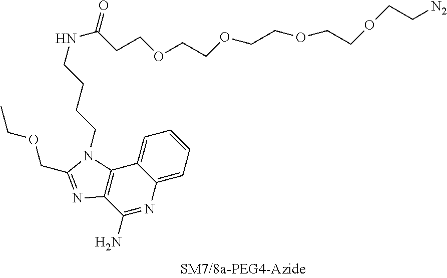

In some embodiments, a PEG4-maleimide linker is conjugated to SM7/8a. An exemplary structure of SM7/8a conjugated to a PEG4-maleimide linker is:

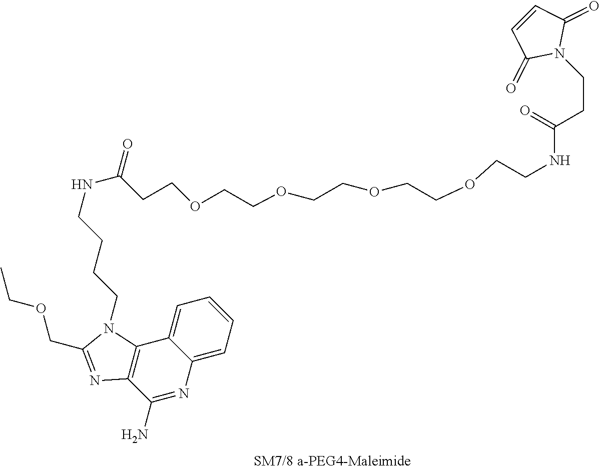 [SMILES: CCOCc1nc2c(N)nc3ccccc3c2n1CCCCNC(=O)CCOCCOCCOCCOCCNC(=O)CCN1C(=O)C=CC1=O]

In some embodiments, an azide group is conjugated to ISS-1018. An exemplary structure of ISS-1018 conjugated to an NHS ester-azide linker is:

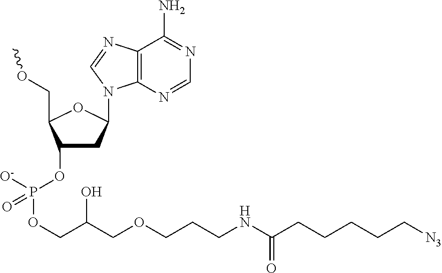

### E. Linkers

In some embodiments, a linker separates the amino acid sequence of the RSV polypeptide from the amino acid sequence of ferritin. Any linker may be used. In some embodiments, the linker is a peptide linker, which can facilitate expression of the antigenic ferritin polypeptide as a fusion protein (e.g., from a single open reading frame). In some embodiments, the linker is a glycine-serine linker. In some embodiments, the glycine-serine linker is GS, GGGS (SEQ ID NO: 226), 2XGGGS (SEQ ID NO: 227) (i.e., GGGSGGGS (SEQ ID NO: 227)), or 5XGGGS (SEQ ID NO: 228). The linker may be N- or C-terminal to ferritin.

In some embodiments, the linker is 2, 3, 4, 5, 6, 7, 8, 9, or 10 amino acids in length. In some embodiments, the linker is about 2-4, 2-6, 2-8, 2-10, 2-12, or 2-14 amino acids in length. In some embodiments, the linker is at least 15 amino acids in length. In some embodiments, the linker is at least 25 amino acids in length. In some embodiments, the linker is at least 30 amino acids in length. In some embodiments, the linker is at least 35 amino acids in length. In some embodiments, the linker is at least 40 amino acids in length. In some embodiments, the linker is less than or equal to 60 amino acids in length. In some embodiments, the linker is less than or equal to 50 amino acids in length. In some embodiments, the linker is about 16, 28, 40, 46, or 47 amino acids in length. In some embodiments, the linker is flexible. In some embodiments, the linker comprises a cysteine, e.g., for use as a site for conjugation of an immune-stimulatory moiety (e.g., adjuvant); an exemplary linker comprising a cysteine is provided as SEQ ID NO: 225. In some embodiments, the linker comprises a sequence with at least 75%, 80%, 85%, 90%, or 95% identity to SEQ ID NO: 225, and further comprises a cysteine corresponding to the cysteine in SEQ ID NO: 225. In some embodiments, the linker comprises at least 25 amino acids (e.g., 25 to 60 amino acids), wherein a cysteine is located at a position ranging from the 8^{th} amino acid from the N-terminus to the 8^{th} amino acid from the C-terminus, or within 10 amino acids of the central residue or bond of the linker.

In some embodiments, the linker comprises glycine (G) and/or serine (S) amino acids. In some embodiments, the linker comprises or consists of glycine (G), serine (S), asparagine (N), and/or alanine (A) amino acids, and optionally a cysteine as discussed above. In some embodiments, the linker comprises an amino acid sequence with at least 80%, 85%, 90%, 95%, 97%, 98%, 99%, or 100% identity to SEQ ID NO: 222. In some embodiments, the linker comprises GGGGSGGGGSGGGGSG (SEQ ID NO: 220), GGSGSGSNSSASSGASSGGASGGSGGSG (SEQ ID NO: 221), GGSGSASSGASASGSSNGSGSGSGSNSSASSGASSGGASGGSGGSG (SEQ ID NO: 222), or GS. In some embodiments, the linker comprises FR1 (SEQ ID NO: 223) or FR2 (SEQ ID NO: 224).

In some embodiments, the ferritin comprises H. pylori ferritin with the amino terminal extension of bullfrog ferritin (which will be referred to as hybrid ferritin). In some embodiments, this hybrid ferritin forms multimers with RSV polypeptide-attachment sites distributed evenly on the surface (see Kanekiyo 2015). In some embodiments, N-terminal fusion proteins with hybrid ferritin allow presentation of an RSV polypeptide on the ferritin nanoparticle surface. In some embodiments, a ferritin comprises a glutamate at a position corresponding to position 13 of SEQ ID NO: 208 (hybrid ferritin, which comprises this glutamate) or position 6 in SEQ ID NO: 209 (wild-type H. pylori ferritin, in which position 6 is isoleucine). In combination with a bullfrog linker, this glutamate is thought to preserve the conserved salt bridge found in human and bullfrog ferritins (6R and 14E in both human light chain and bullfrog lower-subunit ferritins). See Kanekiyo et al., Cell 162, 1090-1100 (2015)).

In some embodiments, an RSV polypeptide is linked to ferritin via a cysteine-thrombin-histidine linker. In some embodiments, this linker is used to directly conjugate a moiety (e.g., immune-stimulatory moiety or RSV polypeptide) to ferritin via click chemistry. An exemplary sequence comprising a cysteine-thrombin-histidine linker is SEQ ID NO: 218. Click chemistry suitable for conjugation reactions involving the cysteine-thrombin-histidine linker is discussed above.

In some embodiments, a linker comprising a cysteine as a conjugation site for an immune-stimulatory moiety such as an adjuvant is used in a construct comprising a ferritin molecule lacking an unpaired, surface-exposed cysteine, or in a construct comprising a ferritin molecule comprising an unpaired, surface-exposed cysteine.

In some embodiments, a construct does not comprise a linker. In some embodiments, a construct comprises one linker. In some embodiments, a construct comprises two or more than two linkers.

### F. Compositions; Uses and Methods for Vaccination

In some embodiments, the present invention provides methods of immunizing a subject against infection with RSV. The present invention further provides methods of eliciting an immune response against RSV in a subject. In some embodiments, the present methods comprise administering to the subject an effective amount of a pharmaceutical composition described herein to a subject. In some embodiments, the present methods comprises administering to the subject an effective amount of an antigenic RSV polypeptide, antigenic ferritin polypeptide, or nanoparticle described herein to a subject.

In some embodiments, a composition comprising any one or more of the polypeptides, nanoparticles, or fusion proteins described herein and a pharmaceutically acceptable vehicle, adjuvant, or excipient is provided.

In some embodiments, a polypeptide, nanoparticle, or composition described herein is administered to a subject, such as a human, to immunize against infection caused by RSV. In some embodiments, a polypeptide or fusion protein described herein is administered to a subject, such as a human, to produce a protective immune response to future infection with RSV. In some embodiments, any one or more of the polypeptides, nanoparticles, or compositions described herein are provided for use in immunizing against infection caused by RSV. In some embodiments, any one or more of the polypeptides, nanoparticles, or compositions described herein are provided for use in producing a protective immune response to future infection with RSV. In some embodiments, the protective immune response decreases the incidence of infection with RSV, pneumonia, bronchiolitis, or asthma

In some embodiments, a composition comprises an RSV F polypeptide described herein. In some embodiments, a composition comprises an RSV G polypeptide described herein. In some embodiments, a composition comprises an RSV F polypeptide described herein and an RSV G polypeptide. In some embodiments, a composition comprises an RSV G polypeptide described herein and an RSV F polypeptide. In some embodiments, a composition comprises an RSV F polypeptide described herein and an RSV G polypeptide described herein.

In some embodiments, a composition comprising an RSV F polypeptide described herein elicits a superior neutralizing response to RSV compared to immunization with a post-fusion RSV F polypeptide. In some embodiments, immunization with an RSV F polypeptide described herein (e.g., a polypeptide or nanoparticle comprising an RSV F polypeptide described herein) elicits a higher titer of antibodies directed against pre-fusion RSV F compared to immunization with a post-fusion RSV F. In some embodiments, immunization with an RSV F polypeptide described herein elicits a lower titer of antibodies directed against post-fusion RSV F compared to immunization with a post-fusion RSV F. In some embodiments, immunization with an RSV F polypeptide described herein elicits a higher ratio of total antibody being directed against pre-fusion RSV F compared to immunization with a post-fusion RSV F. Immunization with an RSV antigen described herein may provide better protection against RSV compared to immunization with a post-fusion RSV F. Epitopes present in post-fusion RSV F, and shared with pre-fusion F, may be non-neutralizing and in some instances have been suggested to elicit antibodies which increase RSV infection. In some embodiments, a composition comprising an RSV F polypeptide described herein elicits a higher neutralizing response to RSV while lessening the antibodies directed against post-fusion RSV F. Thus, in some embodiments, a composition comprising an RSV F polypeptide described herein elicits a higher RSV neutralizing titer to post-fusion F binding response.

In some embodiments, immunization with an RSV antigen described herein yields an improved safety profile compared to immunization with a post-fusion RSV F. This improved safety profile may be related to blocking non-neutralizing epitopes or poorly neutralizing epitopes present on the post-fusion conformation. It has been reported that antibodies which bind the post-fusion conformation may increase RSV infection through antibody mediated viral infection. Thus, post-fusion antibodies that do not significantly neutralize the RSV virus may increase RSV infection, such as those that recognize both the pre-fusion and post-fusion conformation.

In some embodiments, a composition comprising an RSV G polypeptide described herein elicits a neutralizing response to RSV.

In some embodiments, a composition comprising an RSV F and RSV G polypeptide described herein elicits a neutralizing response to RSV. In some embodiments, a composition comprising an RSV F and RSV G polypeptide described herein provides improved protection against RSV, e.g., a higher neutralizing titer than a composition that does not comprise both antigens.

### 1. Subjects

In some embodiments, the subject is a mammal. In some embodiments, the subject is a human.

In some embodiments, the subject is an adult (greater than or equal to 18 years of age). In some embodiments, the subject is a child or adolescent (less than 18 years of age). In some embodiments, the subject is elderly (greater than 60 years of age). In some embodiments, the subject is a non-elderly adult (greater than or equal to 18 years of age and less than or equal to 60 years of age).

In some embodiments, more than one administration of the composition is administered to the subject. In some embodiments, a booster administration improves the immune response.

In some embodiments, any one or more of the antigenic polypeptides, or compositions described herein are for use in a mammal, such as a primate (e.g., non-human primate, such as a monkey (e.g., a macaque, such as rhesus or cynomolgus) or ape), rodent (e.g., mouse or rat), or domesticated mammal (e.g., dog, rabbit, cat, horse, sheep, cow, goat, camel, or donkey). In some embodiments, any one or more of the antigenic polypeptides, or compositions described herein are for use in a bird, such as a fowl (e.g., chicken, turkey, duck, goose, guineafowl, or swan).

### 2. Adjuvants

As described herein, adjuvants may be conjugated to ferritin via a surface exposed amino acid, e.g., a cysteine. Non-conjugated adjuvant may also be administered together with the antigenic ferritin polypeptides described herein to a subject. In some embodiments, administration of adjuvant together with the antigenic ferritin polypeptide produces a higher titer of antibodies against the RSV polypeptide in the subject as compared to administration of the RSV polypeptide alone, or antigenic ferritin polypeptide alone, without the adjuvant. An adjuvant may promote earlier, more potent, or more persistent immune response to the antigenic polypeptide.

In some embodiments, a composition comprises one adjuvant. In some embodiments, a composition comprises more than one adjuvant. In some embodiments, a composition does not comprise an adjuvant.

In some embodiments, an adjuvant comprises aluminum. In some embodiments, an adjuvant is aluminum phosphate. In some embodiments, an adjuvant is Alum (Alyhydrogel '85 2%; Brenntag—Cat #21645-51-2).

In some embodiments, an adjuvant is an organic adjuvant. In some embodiments, an adjuvant is an oil-based adjuvant. In some embodiments, an adjuvant comprises an oil-in-water nanoemulsion.

In some embodiments, an adjuvant comprises squalene. In some embodiments, the adjuvant comprising squalene is Ribi (Sigma adjuvant system Cat #S6322-1v1), Addavax™ MF59, ASO3, or AF03 (see U.S. Pat. No. 9,703,095). In some embodiments, the adjuvant comprising squalene is a nanoemulsion.

In some embodiments, an adjuvant comprises a polyacrylic acid polymer (PAA). In some embodiments, the adjuvant comprising PAA is SPA09 (see WO 2017218819).

In some embodiments, an adjuvant comprises non-metabolizable oils. In some embodiments, the adjuvant is Incomplete Freund's Adjuvant (IFA).

In some embodiments, an adjuvant comprises non-metabolizable oils and killed Mycobacterium tuberculosis. In some embodiments, the adjuvant is Complete Freund's Adjuvant (CFA).

In some embodiments, an adjuvant is a lipopolysaccharide. In some embodiments, an adjuvant is monophosphoryl A (MPL or MPLA).

### 3. Pharmaceutical Compositions

In various embodiments, a pharmaceutical composition comprising an antigenic ferritin polypeptide described herein and/or related entities is provided. In some embodiments, the pharmaceutical composition is an immunogenic composition (e.g., a vaccine) capable of eliciting an immune response such as a protective immune response against a pathogen.

For example, in some embodiments, the pharmaceutical compositions may comprise one or more of the following: (1) an antigenic ferritin protein comprising (i) a mutation replacing a surface-exposed amino acid with a cysteine and (ii) an RSV polypeptide; (2) an antigenic ferritin protein comprising (i) a mutation replacing a surface exposed amino acid with a cysteine and an immune-stimulatory moiety linked to the cysteine; and (ii) an RSV polypeptide; (3) antigenic ferritin protein comprising (i) a surface-exposed cysteine, (ii) a peptide linker N-terminal to the ferritin protein, and (iii) an RSV polypeptide N-terminal to the peptide linker; (4) an antigenic ferritin protein comprising: (i) a mutation replacing a surface exposed amino acid with a cysteine and an immune-stimulatory moiety linked to the cysteine, (ii) a mutation replacing the internal cysteine at position 31 of H. pylori ferritin, or a mutation of an internal cysteine at a position that is analogous to position 31 of a non-H. pylori ferritin as determined by pair-wise or structural alignment, with a non-cysteine amino acid, (iii) a mutation replacing a surface-exposed asparagine with a non-asparagine amino acid, and (iv) an RSV polypeptide; or (5) a ferritin particle comprising any of the foregoing ferritin proteins. In some embodiments, the pharmaceutical composition comprises an antigenic RSV polypeptide comprising an RSV F polypeptide, wherein an epitope of the RSV polypeptide that is shared between pre-fusion RSV F and post-fusion RSV F is blocked, and/or an antigenic RSV polypeptide comprising an RSV F polypeptide, wherein the RSV F polypeptide comprises amino acid residues 62-69 and 196-209 of SEQ ID NO: 26 and an asparagine corresponding to position 328, 348, or 507 of SEQ ID NO: 26, optionally wherein the antigenic RSV polypeptide further comprises ferritin.

In some embodiments, the present invention provides pharmaceutical compositions comprising antibodies or other agents related to the antigenic polypeptides described herein. In an embodiment, the pharmaceutical composition comprises antibodies that bind to and/or compete with an antigenic polypeptide described herein. Alternatively, the antibodies may recognize viral particles comprising the RSV polypeptide component of an antigenic polypeptide described herein.

In some embodiments, the pharmaceutical compositions as described herein are administered alone or in combination with one or more agents to enhance an immune response, e.g., an adjuvant described above. In some embodiments, a pharmaceutical composition further comprises an adjuvant described above.

In some embodiments, the pharmaceutical composition further comprises a pharmaceutically acceptable carrier or excipient. As used herein, the term “carrier” refers to a diluent, adjuvant, excipient, or vehicle with which a pharmaceutical composition is administered. In exemplary embodiments, carriers can include sterile liquids, such as, for example, water and oils, including oils of petroleum, animal, vegetable, or synthetic origin, such as, for example, peanut oil, soybean oil, mineral oil, sesame oil and the like. In some embodiments, carriers are or include one or more solid components. Pharmaceutically acceptable carriers can also include, but are not limited to, saline, buffered saline, dextrose, glycerol, ethanol, and combinations thereof. As used herein, an excipient is any non-therapeutic agent that may be included in a pharmaceutical composition, for example to provide or contribute to a desired consistency or stabilizing effect. Suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In various embodiments, the pharmaceutical composition is sterile.

In some embodiments, the pharmaceutical composition contains minor amounts of wetting or emulsifying agents, or pH buffering agents. In some embodiments, the pharmaceutical compositions of may include any of a variety of additives, such as stabilizers, buffers, or preservatives. In addition, auxiliary, stabilizing, thickening, lubricating, and coloring agents can be included.

In various embodiments, the pharmaceutical composition may be formulated to suit any desired mode of administration. For example, the pharmaceutical composition can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. General considerations in the formulation and manufacture of pharmaceutical agents may be found, for example, in Remington's Pharmaceutical Sciences, 19th ed., Mack Publishing Co., Easton, PA, 1995; incorporated herein by reference.

The pharmaceutical composition can be administered via any route of administration. Routes of administration include, for example, oral, intradermal, intramuscular, intraperitoneal, intravenous, subcutaneous, intranasal, mucosal, epidural, sublingual, intranasal, intracerebral, intravaginal, transdermal, rectally, by intratracheal instillation, bronchial instillation, inhalation, or topically. Administration can be local or systemic. In some embodiments, administration is carried out orally. In another embodiment, the administration is by parenteral injection. In some instances, administration results in the release of the antigenic ferritin polypeptide described herein into the bloodstream. The mode of administration can be left to the discretion of the practitioner.

In some embodiments, the pharmaceutical composition is suitable for parenteral administration (e.g. intravenous, intramuscular, intraperitoneal, and subcutaneous). Such compositions can be formulated as, for example, solutions, suspensions, dispersions, emulsions, and the like. They may also be manufactured in the form of sterile solid compositions (e.g. lyophilized composition), which can be dissolved or suspended in sterile injectable medium immediately before use. For example, parenteral administration can be achieved by injection. In such embodiments, injectables are prepared in conventional forms, i.e., either as liquid solutions or suspensions, solid forms suitable for solution or suspension in liquid prior to injection, or as emulsions. In some embodiments, injection solutions and suspensions are prepared from sterile powders, lyophilized powders, or granules.

In a further embodiment, the pharmaceutical composition is formulated for delivery by inhalation (e.g., for direct delivery to the lungs and the respiratory system). For example, the composition may take the form of a nasal spray or any other known aerosol formulation. In some embodiments, preparations for inhaled or aerosol delivery comprise a plurality of particles. In some embodiments, such preparations can have a mean particle size of about 1, about 2, about 3, about 4, about 5, about 6, about 7, about 8, about 9, about 10, about 11, about 12, or about 13 microns. In some embodiments, preparations for inhaled or aerosol delivery are formulated as a dry powder. In some embodiments, preparations for inhaled or aerosol delivery are formulated as a wet powder, for example through inclusion of a wetting agent. In some embodiments, the wetting agent is selected from the group consisting of water, saline, or other liquid of physiological pH.

In some embodiments, the pharmaceutical composition in accordance with the invention are administered as drops to the nasal or buccal cavity. In some embodiments, a dose may comprise a plurality of drops (e.g., 1-100, 1-50, 1-20, 1-10, 1-5, etc.).

The present pharmaceutical composition may be administered in any dose appropriate to achieve a desired outcome. In some embodiments, the desired outcome is the induction of a long-lasting adaptive immune response against the source of an RSV polypeptide present in an antigenic ferritin polypeptide present in the composition. In some embodiments, the desired outcome is a reduction in the intensity, severity, frequency, and/or delay of onset of one or more symptoms of infection. In some embodiments, the desired outcome is the inhibition or prevention of infection. The dose required will vary from subject to subject depending on the species, age, weight, and general condition of the subject, the severity of the infection being prevented or treated, the particular composition being used, and its mode of administration.

In some embodiments, pharmaceutical compositions in accordance with the invention are administered in single or multiple doses. In some embodiments, the pharmaceutical compositions are administered in multiple doses administered on different days (e.g., prime-boost vaccination strategies). In some embodiments, the pharmaceutical composition is administered as part of a booster regimen.

In various embodiments, the pharmaceutical composition is co-administered with one or more additional therapeutic agents. Co-administration does not require the therapeutic agents to be administered simultaneously, if the timing of their administration is such that the pharmacological activities of the additional therapeutic agent and the active ingredient(s) in the pharmaceutical composition overlap in time, thereby exerting a combined therapeutic effect. In general, each agent will be administered at a dose and on a time schedule determined for that agent.

4. Nucleic acid/mRNA

Also provided is a nucleic acid encoding an antigenic polypeptide described herein. In some embodiments, the nucleic acid is an mRNA. Any nucleic acid capable of undergoing translation resulting in a polypeptide is considered an mRNA for purposes of this disclosure.

### 5. Kits

Also provided herein are kits comprising one or more antigenic polypeptides, nucleic acids, antigenic ferritin particles, antigenic lumazine synthase particles, compositions, or pharmaceutical compositions described herein. In some embodiments, a kit further comprises one or more of a solvent, solution, buffer, instructions, or desiccant.

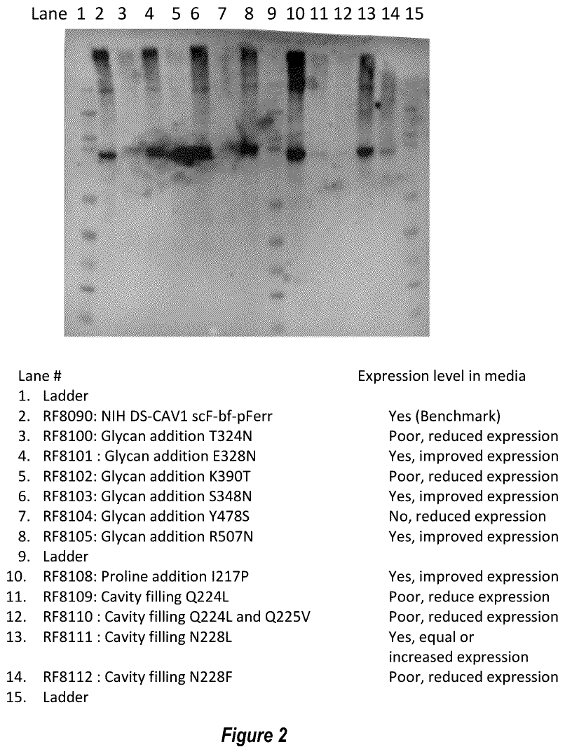

TABLE 1
(Sequence Table): Description of the Sequences

| | | SEQ |
|---|---|---|
| | | ID |
| Description | Sequence | NO |
| RF8085 NIH DS- | MELLILKANAITTILTAVTFCFASGQNITEEFYQSTCSAVSKGYLSALRTGWYTSVITIELSNIKEN | 1 |
| CAV1 with single | KCNGTDAKVKLIKQELDKYKNAVTELQLLMGSGNVGLGGAIASGVAVCKVLHLEGEVNKIKSALLST | |
| chain linker | NKAVVSLSNGVSVLTFKVLDLKNYIDKQLLPILNKQSCSISNIETVIEFQQKNNRLLEITREFSVNA | |
| SGSGS (SEQ ID | GVTTPVSTYMLTNSELLSLINDMPITNDQKKLMSNNVQIVRQQSYSIMCIIKEEVLAYVVQLPLYGV | |
| NO: 229) on | IDTPCWKLHTSPLCTTNTKEGSNICLTRTDRGWYCDNAGSVSFFPQAETCKVQSNRVFCDTMNSRTL | |
| bullfrog (bf) hp | PSEVNLCNVDIFNPKYDCKIMTSKTDVSSSVITSLGAIVSCYGKTKCTASNKNRGIIKTFSNGCDYV | |
| ferritin | SNKGVDTVSVGNTLYYVNKQEGKSLYVKGEPIINFYDPLVFPSDEFDASISQVNEKINQSLAFIRKS | |
| N19Q_C31S_S111C | DELLSGSGSESQVRQQFSKDIEKLLNEQVNKEMQSSNLYMSMSSWSYTHSLDGAGLFLFDHAAEEYE | |
| (control)(same | HAKKLIIFLNENNVPVQLTSISAPEHKFEGLTQIFQKAYEHEQHISESINNIVDHAIKCKDHATFNF | |
| protein sequence | LQWYVAEQHEEEVLFKDILDKIELIGNENHGLYLADQYVKGIAKSRKS | |
| as 2, expressed | | |
| with transient | | |
| transfection | | |
| cloning vector) | | |
| RF8090: NIH DS- | MELLILKANAITTILTAVTFCFASGQNITEEFYQSTCSAVSKGYLSALRTGWYTSVITIELSNIKEN | 2 |
| CAV1 with single | KCNGTDAKVKLIKQELDKYKNAVTELQLLMGSGNVGLGGAIASGVAVCKVLHLEGEVNKIKSALLST | |
| chain linker | NKAVVSLSNGVSVLTFKVLDLKNYIDKQLLPILNKQSCSISNIETVIEFQQKNNRLLEITREFSVNA | |
| SGSGS (SEQ ID | GVTTPVSTYMLTNSELLSLINDMPITNDQKKLMSNNVQIVRQQSYSIMCIIKEEVLAYVVQLPLYGV | |
| NO: 229) on | IDTPCWKLHTSPLCTTNTKEGSNICLTRTDRGWYCDNAGSVSFFPQAETCKVQSNRVFCDTMNSRTL | |
| bullfrog (bf) hp | PSEVNLCNVDIFNPKYDCKIMTSKTDVSSSVITSLGAIVSCYGKTKCTASNKNRGIIKTFSNGCDYV | |
| ferritin | SNKGVDTVSVGNTLYYVNKQEGKSLYVKGEPIINFYDPLVFPSDEFDASISQVNEKINQSLAFIRKS | |
| N19Q_C31S_S111C | DELLSGSGSESQVRQQFSKDIEKLLNEQVNKEMQSSNLYMSMSSWSYTHSLDGAGLFLFDHAAEEYE | |
| (control)(same | HAKKLIIFLNENNVPVQLTSISAPEHKFEGLTQIFQKAYEHEQHISESINNIVDHAIKCKDHATFNF | |
| protein sequence | LQWYVAEQHEEEVLFKDILDKIELIGNENHGLYLADQYVKGIAKSRKS | |
| as 1, expressed | | |
| with cloning | | |
| vector used for | | |
| CHO cell line | | |
| generation | | |
| RF8100: Add a | MELLILKANAITTILTAVTFCFASGQNITEEFYQSTCSAVSKGYLSALRTGWYTSVITIELSNIKEN | 3 |
| single T324N | KCNGTDAKVKLIKQELDKYKNAVTELQLLMGSGNVGLGGAIASGVAVCKVLHLEGEVNKIKSALLST | |
| glycan site to | NKAVVSLSNGVSVLTFKVLDLKNYIDKQLLPILNKQSCSISNIETVIEFQQKNNRLLEITREFSVNA | |
| RSV scF_SGSGS- | GVTTPVSTYMLTNSELLSLINDMPITNDQKKLMSNNVQIVRQQSYSIMCIIKEEVLAYVVQLPLYGV | |
| bf- | IDTPCWKLHTSPLCTNNTKEGSNICLTRTDRGWYCDNAGSVSFFPQAETCKVQSNRVFCDTMNSRTL | |
| pFerr_N19Q_C31S | PSEVNLCNVDIFNPKYDCKIMTSKTDVSSSVITSLGAIVSCYGKTKCTASNKNRGIIKTFSNGCDYV | |
| S111C] | SNKGVDTVSVGNTLYYVNKQEGKSLYVKGEPIINFYDPLVFPSDEFDASISQVNEKINQSLAFIRKS | |
| | DELLSGSGSESQVRQQFSKDIEKLLNEQVNKEMQSSNLYMSMSSWSYTHSLDGAGLFLFDHAAEEYE | |
| | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8101: Add a | MELLILKANAITTILTAVTFCFASGQNITEEFYQSTCSAVSKGYLSALRTGWYTSVITIELSNIKEN | 4 |
| single glycan | KCNGTDAKVKLIKQELDKYKNAVTELQLLMGSGNVGLGGAIASGVAVCKVLHLEGEVNKIKSALLST | |
| site E328N to | NKAVVSLSNGVSVLTFKVLDLKNYIDKQLLPILNKQSCSISNIETVIEFQQKNNRLLEITREFSVNA | |
| RSV scF_SGSGS- | GVTTPVSTYMLTNSELLSLINDMPITNDQKKLMSNNVQIVRQQSYSIMCIIKEEVLAYVVQLPLYGV | |
| bf- | IDTPCWKLHTSPLCTTNTKNGSNICLTRTDRGWYCDNAGSVSFFPQAETCKVQSNRVFCDTMNSRTL | |
| pFerr_N19Q_C31S | PSEVNLCNVDIFNPKYDCKIMTSKTDVSSSVITSLGAIVSCYGKTKCTASNKNRGIIKTFSNGCDYV | |
| | SNKGVDTVSVGNTLYYVNKQEGKSLYVKGEPIINFYDPLVFPSDEFDASISQVNEKINQSLAFIRKS | |
| | DELLSGSGSESQVRQQFSKDIEKLLNEQVNKEMQSSNLYMSMSSWSYTHSLDGAGLFLFDHAAEEYE | |
| | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8102: Add a | MELLILKANAITTILTAVTFCFASGQNITEEFYQSTCSAVSKGYLSALRTGWYTSVITIELSNIKEN | 5 |
| single glycan | KCNGTDAKVKLIKQELDKYKNAVTELQLLMGSGNVGLGGAIASGVAVCKVLHLEGEVNKIKSALLST | |
| site K3901 to | NKAVVSLSNGVSVLTFKVLDLKNYIDKQLLPILNKQSCSISNIETVIEFQQKNNRLLEITREFSVNA | |
| RSV scF_SGSGS- | GVTTPVSTYMLTNSELLSLINDMPITNDQKKLMSNNVQIVRQQSYSIMCIIKEEVLAYVVQLPLYGV | |
| bf- | IDTPCWKLHTSPLCTTNTKEGSNICLTRTDRGWYCDNAGSVSFFPQAETCKVQSNRVFCDTMNSRTL | |
| pFerr_N19Q_C31S | PSEVNLCNVDIFNPTYDCKIMTSKTDVSSSVITSLGAIVSCYGKTKCTASNKNRGIIKTFSNGCDYV | |
| S111C | SNKGVDTVSVGNTLYYVNKQEGKSLYVKGEPIINFYDPLVFPSDEFDASISQVNEKINQSLAFIRKS | |
| | DELLSGSGSESQVRQQFSKDIEKLLNEQVNKEMQSSNLYMSMSSWSYTHSLDGAGLFLFDHAAEEYE | |
| | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8103: Add a | MELLILKANAITTILTAVTFCFASGQNITEEFYQSTCSAVSKGYLSALRTGWYTSVITIELSNIKEN | 6 |
| single glycan | AKCNGTDAKVKLIKQELDKYKNVTELQLLMGSGNVGLGGAIASGVAVCKVLHLEGEVNKIKSALLST | |
| site S348N to | NKAVVSLSNGVSVLTFKVLDLKNYIDKQLLPILNKQSCSISNIETVIEFQQKNNRLLEITREFSVNA | |
| RSVscF_SGSGS-bf- | GVTTPVSTYMLTNSELLSLINDMPITNDQKKLMSNNVQIVRQQSYSIMCIIKEEVLAYVVQLPLYGV | |
| pFerr_N19Q_C31S | CIDTPCWKLHTSPLCTTNTKEGSNICLTRTDRGWYCDNAGNVSFFPQAETCKVQSNRVFCDTMNSRT | |
| S111C | LPSEVNLCNVDIFNPKYDKIMTSKTDVSSSVITSLGAIVSCYGKTKCTASNKNRGIIKTFSNGCDYV | |
| | SNKGVDTVSVGNTLYYVNKQEGKSLYVKGEPIINFYDPLVFPSDEFDASISQVNEKINQSLAFIRKS | |
| | DELLSGSGSESQVRQQFSKDIEKLLNEQVNKEMQSSNLYMSMSSWSYTHSLDGAGLFLFDHAAEEYE | |
| | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8104: Add a | MELLILKANAITTILTAVTFCFASGQNITEEFYQSTCSAVSKGYLSALRTGWYTSVITIELSNIKEN | 7 |
| single glycan | KCNGTDAKVKLIKQELDKYKNAVTELQLLMGSGNVGLGGAIASGVAVCKVLHLEGEVNKIKSALLST | |
| site Y478S to | NKAVVSLSNGVSVLTFKVLDLKNYIDKQLLPILNKQSCSISNIETVIEFQQKNNRLLEITREFSVNA | |
| RSV scF_SGSGS- | GVTTPVSTYMLTNSELLSLINDMPITNDQKKLMSNNVQIVRQQSYSIMCIIKEEVLAYVVQLPLYGV | |
| bf- | IDTPCWKLHTSPLCTTNTKEGSNICLTRTDRGWYCDNAGSVSFFPQAETCKVQSNRVFCDTMNSRTL | |
| pFerr_N19Q_C31S | PSEVNLCNVDIFNPKYDCKIMTSKTDVSSSVITSLGAIVSCYGKTKCTASNKNRGIIKTFSNGCDYV | |
| S1 11C | SNKGVDTVSVGNTLYYVNKQEGKSLYVKGEPIINFSDPLVFPSDEFDASISQVNEKINQSLAFIRKS | |
| | DELLSGSGSESQVRQQFSKDIEKLLNEQVNKEMQSSNLYMSMSSWSYTHSLDGAGLFLFDHAAEEYE | |
| | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8105: Add a | MELLILKANAITTILTAVTFCFASGQNITEEFYQSTCSAVSKGYLSALRTGWYTSVITIELSNIKEN | 8 |
| single glycan | KCNGTDAKVKLIKQELDKYKNAVTELQLLMGSGNVGLGGAIASGVAVCKVLHLEGEVNKIKSALLST | |
| site R507N to | NKAVVSLSNGVSVLTFKVLDLKNYIDKQLLPILNKQSCSISNIETVIEFQQKNNRLLEITREFSVNA | |
| RSV scF_SGSGS- | GVTTPVSTYMLTNSELLSLINDMPITNDQKKLMSNNVQIVRQQSYSIMCIIKEEVLAYVVQLPLYGV | |
| bf- | IDTPCWKLHTSPLCTTNTKEGSNICLTRTDRGWYCDNAGSVSFFPQAETCKVQSNRVFCDTMNSRTL | |
| pFerr_N19Q_C31S | PSEVNLCNVDIFNPKYDCKIMTSKTDVSSSVITSLGAIVSCYGKTKCTASNKNRGIIKTFSNGCDYV | |
| S111C | SNKGVDTVSVGNTLYYVNKQEGKSLYVKGEPIINFYDPLVFPSDEFDASISQVNEKINQSLAFINKS | |
| | DELLSGSGSESQVRQQFSKDIEKLLNEQVNKEMQSSNLYMSMSSWSYTHSLDGAGLFLFDHAAEEYE | |
| | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8106: RF8108 | mellilkanaittiltavtfcfasgqniteefyqstcsayskgylsalrtgwytsvitielsniken | 9 |
| with I217P that | kongtdakvklikqeldkyknavtelqllmgsgnvglggaiasgvayskvlhlegevnkiksallst | |
| increases | nkavvslsngvsvltfkvldlknyidkqllpilnkqscsisnpetviefqqknnrlleitrefsvna | |
| expression, and | gvttpvstymltnsellslindmpitndqkklmsnnvqivrqqsysimsiikeevlayvvqlplygv | |
| removal of | idtpcwklhtsplottntkegsnicltrtdrgwycdnagsysffpqaetckvqsnrvfcdtmnsrtl | |
| disulfide bond | psevnlonvdifnpkydckimtsktdvsssvitslgaivscygktkotasnknrgiiktfsngodyv | |
| (DS) of DS-CAV1 | snkgvdtvsvgntlyyvnkqegkslyvkgepiinfydplvfpsdefdasisqvnekingslafirks | |
| removed, | dellsgsgsesqvrqqfskdiekllneqvnkemqssnlymsmsswsythsldgaglflfdhaaeeye | |
| resulting in | hakkliiflnennvpvqltsisapehkfegltqifqkayeheqhisesinnivdhaikckdhatfnf | |
| even higher | lqwyvaeqheeevlfkdildkielignenhglyladqyvkgiaksrks | |
| expression | | |
| relative to | | |
| RF8085 (or | | |
| RF8090) | | |
| RF8107: Proline | mellilkanaittiltavtfcfasgqniteefyqstcsayskgylsalrtgwytsvitielsniken | 10 |
| substitution | kongtdakvklikqeldkyknavtelqllmgsgnvglggaiasgvayskvlhlegevnkiksallst | |
| (I217P) of | nkavvslsngvsvltfkvldlknyidkqllpilnkqscsisnpetviefqqknlrlleitrefsvna | |
| RF8108 and | gvttpvstymltnsellslindmpitndqkklmsnnvqivrqqsysimsiikeevlayvvqlplygv | |
| hydrophobic | idtpcwklhtsplottntkegsnicltrtdrgwycdnagsysffpqaetckvqsnrvfcdtmnsrtl | |
| cavity filling | psevnlonvdifnpkydckimtsktdvsssvitslgaivscygktkotasnknrgiiktfsngodyv | |
| substitution of | snkgvdtvsvgntlyyvnkqegkslyvkgepiinfydplvfpsdefdasisqvnekingslafirks | |
| RF8111 (N228L) | dellsgsgsesqvrqqfskdiekllneqvnkemqssnlymsmsswsythsldgaglflfdhaaeeye | |
| together with | hakkliiflnennvpvqltsisapehkfegltqifqkayeheqhisesinnivdhaikckdhatfnf | |
| removal of | lqwyvaeqheeevlfkdildkielignenhglyladqyvkgiaksrks | |
| disulfide bond | | |
| of DS-CAV1 | | |
| RF8108: Proline | MELLILKANAITTILTAVTFCFASGQNITEEFYQSTCSAVSKGYLSALRTGWYTSVITIELSNIKEN | 11 |
| substitution | KCNGTDAKVKLIKQELDKYKNAVTELQLLMGSGNVGLGGAIASGVAVCKVLHLEGEVNKIKSALLST | |
| I217P to | NKAVVSLSNGVSVLTFKVLDLKNYIDKQLLPILNKQSCSISNPETVIEFQQKNNRLLEITREFSVNA | |
| stabilize pre- | GVTTPVSTYMLTNSELLSLINDMPITNDQKKLMSNNVQIVRQQSYSIMCIIKEEVLAYVVQLPLYGV | |
| fusion central | IDTPCWKLHTSPLCTTNTKEGSNICLTRTDRGWYCDNAGSVSFFPQAETCKVQSNRVFCDTMNSRTL | |
| helix (while DS | PSEVNLCNVDIFNPKYDCKIMTSKTDVSSSVITSLGAIVSCYGKTKCTASNKNRGIIKTFSNGCDYV | |
| of DS-CAV1 | SNKGVDTVSVGNTLYYVNKQEGKSLYVKGEPIINFYDPLVFPSDEFDASISQVNEKINQSLAFIRKS | |
| present) and | DELLSGSGSESQVRQQFSKDIEKLLNEQVNKEMQSSNLYMSMSSWSYTHSLDGAGLFLFDHAAEEYE | |
| increase | HAKKLIIFLNENNVPVQLTSISAPEHKFEGLTQIFQKAYEHEQHISESINNIVDHAIKCKDHATFNF | |
| expression | LQWYVAEQHEEEVLFKDILDKIELIGNENHGLYLADQYVKGIAKSRKS | |
| RF8109: | MELLILKANAITTILTAVTFCFASGQNITEEFYQSTCSAVSKGYLSALRTGWYTSVITIELSNIKEN | 12 |
| Hydrophobic | KCNGTDAKVKLIKQELDKYKNAVTELQLLMGSGNVGLGGAIASGVAVCKVLHLEGEVNKIKSALLST | |
| cavity filling | NKAVVSLSNGVSVLTFKVLDLKNYIDKQLLPILNKQSCSISNIETVIEFLQKNNRLLEITREFSVNA | |
| Q224L on RSV | GVTTPVSTYMLTNSELLSLINDMPITNDQKKLMSNNVQIVRQQSYSIMCIIKEEVLAYVVQLPLYGV | |
| scF_SGSGS-bf- | IDTPCWKLHTSPLCTTNTKEGSNICLTRTDRGWYCDNAGSVSFFPQAETCKVQSNRVFCDTMNSRTL | |
| pFerr_N19Q_C31S | PSEVNLCNVDIFNPKYDCKIMTSKTDVSSSVITSLGAIVSCYGKTKCTASNKNRGIIKTFSNGCDYV | |
| S111C | SNKGVDTVSVGNTLYYVNKQEGKSLYVKGEPIINFYDPLVFPSDEFDASISQVNEKINQSLAFIRKS | |
| | DELLSGSGSESQVRQQFSKDIEKLLNEQVNKEMQSSNLYMSMSSWSYTHSLDGAGLFLFDHAAEEYE | |
| | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8110: | MELLILKANAITTILTAVTFCFASGQNITEEFYQSTCSAVSKGYLSALRTGWYTSVITIELSNIKEN | 13 |
| Hydrophobic | KCNGTDAKVKLIKQELDKYKNAVTELQLLMGSGNVGLGGAIASGVAVCKVLHLEGEVNKIKSALLST | |
| cavity filling | NKAVVSLSNGVSVLTFKVLDLKNYIDKQLLPILNKQSCSISNIETVIEFLVKNNRLLEITREFSVNA | |
| substitutions | GVTTPVSTYMLTNSELLSLINDMPITNDQKKLMSNNVQIVRQQSYSIMCIIKEEVLAYVVQLPLYGV | |
| Q224L and Q225V | IDTPCWKLHTSPLCTTNTKEGSNICLTRTDRGWYCDNAGSVSFFPQAETCKVQSNRVFCDTMNSRTL | |
| on RSV | PSEVNLCNVDIFNPKYDCKIMTSKTDVSSSVITSLGAIVSCYGKTKCTASNKNRGIIKTFSNGCDYV | |
| scF_SGSGS-bf- | SNKGVDTVSVGNTLYYVNKQEGKSLYVKGEPIINFYDPLVFPSDEFDASISQVNEKINQSLAFIRKS | |
| pFerr_N19Q_C31S | DELLSGSGSESQVRQQFSKDIEKLLNEQVNKEMQSSNLYMSMSSWSYTHSLDGAGLFLFDHAAEEYE | |
| S111C | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8111: | MELLILKANAITTILTAVTFCFASGQNITEEFYQSTCSAVSKGYLSALRTGWYTSVITIELSNIKEN | 14 |
| Hydrophobic | KCNGTDAKVKLIKQELDKYKNAVTELQLLMGSGNVGLGGAIASGVAVCKVLHLEGEVNKIKSALLST | |
| cavity filling | NKAVVSLSNGVSVLTFKVLDLKNYIDKQLLPILNKQSCSISNIETVIEFQQKNLRLLEITREFSVNA | |
| substitution | GVTTPVSTYMLTNSELLSLINDMPITNDQKKLMSNNVQIVRQQSYSIMCIIKEEVLAYVVQLPLYGV | |
| N228L on RSV | IDTPCWKLHTSPLCTTNTKEGSNICLTRTDRGWYCDNAGSVSFFPQAETCKVQSNRVFCDTMNSRTL | |
| scF_SGSGS-bf- | PSEVNLCNVDIFNPKYDCKIMTSKTDVSSSVITSLGAIVSCYGKTKCTASNKNRGIIKTFSNGCDYV | |
| pFerr_N19Q_C31S | SNKGVDTVSVGNTLYYVNKQEGKSLYVKGEPIINFYDPLVFPSDEFDASISQVNEKINQSLAFIRKS | |
| S111C | DELLSGSGSESQVRQQFSKDIEKLLNEQVNKEMQSSNLYMSMSSWSYTHSLDGAGLFLFDHAAEEYE | |
| | HAKKLIIFLNENNVPVQLTSISAPEHKFEGLTQIFQKAYEHEQHISESINNIVDHAIKCKDHATFNF | |
| | LQWYVAEQHEEEVLFKDILDKIELIGNENHGLYLADQYVKGIAKSRKS | |
| RF8112 | MELLILKANAITTILTAVTFCFASGQNITEEFYQSTCSAVSKGYLSALRTGWYTSVITIELSNIKENK | 15 |
| hydrophobic | CNGTDAKVKLIKQELDKYKNAVTELQLLMGSGNVGLGGAIASGVAVCKVLHLEGEVNKIKSALLSTNK | |
| filling | AVVSLSNGVSVLTFKVLDLKNYIDEQLLPILNKQSCSISNIETVIEFQQKNFRLLEITREFSVNAGVT | |
| substitution | TPVSTYMLTNSELLSLINDMPITNDQKKLMSNNVQIVRQQSYSIMCIIKEEVLAYVVQLPLYGVIDTP | |
| N228F on RSV | CWELHTSPLCTTNTKEGSNICLTRTDRGWYCDNAGSVSFFPQAETCKVQSNRVFCDTMNSRTLPSEVN | |
| scF_SGSGS-bf- | LCNVDIFNPKYDCKIMTSKTDVSSSVITSLGAIVSCYGETECTASNENRGIIKTFSNGCDYVSNEGVD | |
| pFerr_N19Q_C31S | TVSVGNTLYYVNKQEGKSLYVKGEPIINFYDPLVFPSDEFDASISQVNEKINQSLAFIRKSDELLSGS | |
| S111C | GSESQVRQQFSEDIEKLLNEQVNKEMQSSNLYMSMSSWSYTHSLDGAGLFLFDHAAEEYEHAKKLIIF | |
| | LNENNVPVQLTSISAPEHEFEGLTQIFQKAYEHEQHISESINNIVDHAIKCEDHATFNFLQWYVAEQH | |
| | EEEVLFEDILDKIELIGNENHGLYLADQYVEGIAKSRKS | |
| RF8113 (1217P | mellilkanaittiltavtfcfasgqniteefyqstcsayskgylsalrtgwytsvitielsnikenk | 16 |
| mutation of | ongtdakvklikqeldkyknavtelqllmgsgnvglggaiasgvayskvlhlegevnkiksallstnk | |
| RF8106 while | pavvslsngvsvltfkvldlknyidkqllpilnkqscsisnetviefqqknnrlleitrefsvnagvt | |
| removing DS of | tpvstymltnsellslindmpitndqkklmsnnvqivrqqsysimsiikeevlayvvqlplygvidtp | |
| DS-CAV1 and with | cwklhtsplottntkegsnicltrtdrgwycdnagsysffpqaetckvqsnrvfcdtmnsrtlpsevn | |
| ferritin Wt | lonvdifnpkydckimtsktdvsssvitslgaivscygktkotasnknrgiiktfsngodyvsnkgvd | |
| ser111 and | tvsvgntlyyvnkqegkslyvkgepiinfydplvfpsdefdasisqvnekingslafirksdellsgs | |
| engineered CYS | gsesqvrqqfskdiekllneqvnkemqssnlymsmsswsythsldgaglflfdhaaeeyehakkliif | |
| of K79C for | lnennvpvqltsisapehcfegltqifqkayeheqhisesinnivdhaikskdhatfnflqwyvaeqh | |
| conjugation) | eeevlfkdildkielignenhglyladqyvkgiaksrks | |
| RF8117 | mellilkanaittiltavtfcfasgqniteefyqstcsayskgylsalrtgwytsvitielsnikenk | 17 |
| (Combinations of | ongtdakvklikqeldkyknavtelqllmgsgnvglggaiasgvayskvlhlegevnkiksallstnk | |
| above successful | avvslsngvsvltfkvldlknyidkqllpilnkqscsisnpetviefqqknnrlleitrefsvnagvt | |
| improved | tpvstymltnsellslindmpitndqkklmsnnvqivrqqsysimsiikeevlayvvqlplygvidtp | |
| expression/ | cwklhtsplottntkngsnicltrtdrgwycdnagnvsffpqaetckvqsnrvfcdtmnsrtlpsevn | |
| secretion | lonvdifnpkydckimtsktdvsssvitslgaivscygktkotasnknrgiiktfsngodyvsnkgvd | |
| mutations | tvsvgntlyyvnkqegkslyvkgepiinfydplvfpsdefdasisqvnekingslafinksdellsgs | |
| above (FIG. 2): | lgsesqvrqqfskdiekllneqvnkemqssnlymsmsswsythsldgaglflfdhaaeeyehakklii | |
| No DS, I217P, | flnennvpvqltsisapehCfegltqifqkayeheqhisesinnivdhaikskdhatfnflqwyvaeq | |
| E328N, 5348N, | heeevfkdildkielignenhglyladqyvkgiaksrks | |
| R507N, | | |
| ferritinK79C) | | |
| RF8122 (RF8117 | mellilkanaittiltavtfcfasgqniteefyqstcsayskgylsalrtgwytsvitielsnikenk | 18 |
| above with | ongtdakvklikqeldkyknavtelqllmgsgnvglggaiasgvayskvlhlegevnkiksallstnk | |
| additional K498L | avvslsngvsvltfkvldlknyidkqllpilnkqscsisnpetviefqqknnrlleitrefsvnagvt | |
| and K508Q for | tpvstymltnsellslindmpitndqkklmsnnvqivrqqsysimsiikeevlayvvqlplygvidtp | |
| removing | cwklhtsplottntkngsnicltrtdrgwycdnagnvsffpqaetckvqsnrvfcdtmnsrtlpsevn | |
| protease (LYS- | lonvdifnpkydckimtsktdvsssvitslgaivscygktkotasnknrgiiktfsngodyvsnkgvd | |
| based) cleavage | tvsvgntlyyvnkqegkslyvkgepiinfydplvfpsdefdasisqvnelingslafingsdellsgs | |
| and increased | gsesqvrqqfskdiekllneqvnkemqssnlymsmsswsythsldgaglflfdhaaeeyehakkliif | |
| stability/ | lnennvpvqltsisapehcfegltqifqkayeheqhisesinnivdhaikskdhatfnflqwyvaeqh | |
| expression) | eeevlfkdildkielignenhglyladqyvkgiaksrks | |
| RF8123 (RF8117 | mellilkanaittiltavtfcfasgqniteefyqstcsayskgylsalrtgwytsvitielsnikenk | 19 |
| with C's at 69 | vngtdakvklikqeldkyknavtelqllmgsgnvglggaiasgvayskvlhlegevnkiksallstnk | |
| and 212 knocked | avvslsngvsvltfkvldlknyidkqllpilnkqsysisnpetviefqqknnrlleitrefsvnagvt | |
| out for specific | tpvstymltnsellslindmpitndqkklmsnnvqivrqqsysimsiikeevlayvvqlplygvidtp | |
| conjugation to | cwklhtsplottntkngsnicltrtdrgwycdnagnvsffpqaetckvqsnrvfcdtmnsrtlpsevn | |
| ferritin CYS: | lonvdifnpkydckimtsktdvsssvitslgaivscygktkotasnknrgiiktfsngodyvsnkgvd | |
| C69V, C212V) | tvsvgntlyyvnkqegkslyvkgepiinfydplvfpsdefdasisqvnekingslafinksdellsgs | |
| | gsesqvrqqfskdiekllneqvnkemqssnlymsmsswsythsldgaglflfdhaaeeyehakkliif | |
| | lnennvpvqltsisapehcfegltqifqkayeheqhisesinnivdhaikskdhatfnflqwyvaeqh | |
| | eeevlfkdildkielignenhglyladqyvkgiaksrks | |
| RF8134 (RF8122 | mellilkanaittiltavtfcfasgqniteefyqstcsayskgylsalrtgwytsvitielsnikenk | 20 |
| like with K52 8N | ongtdakvklikqeldkyknavtelqllmgsgnvglggaiasgvayskvlhlegevnkiksallstnk | |
| and K532N | avvslsngvsvltfkvldlknyidkqllpilnkqscsisnpetviefqqknnrlleitrefsvnagvt | |
| mutations to | ltpvstymltnsellslindmpitndqkklmsnnvqivrqqsysimsiikeevlayvvqlpygvidtp | |
| limit | cwklhtsplottntkngsnicltrtdrgwycdnagnvsffpqaetckvqsnrvfcdtmnsrtlpsevn | |
| proteolysis | lonvdifnpkydckimtsktdvsssvitslgaivscygktkotasnknrgiiktfsngodyvsnkgvd | |
| instability | tvsvgntlyyvnkqegkslyvkgepiinfydplvfpsdefdasisqvnelingslafingsdellsgs | |
| | gsesqvrqqfsndienllneqvnkemqssnlymsmsswsythsldgaglflfdhaaeeyehakkliif | |
| | lnennvpvqltsisapehcfegltqifqkayeheqhisesinnivdhaikskdhatfnflqwyvaeqh | |
| | eeevlfkdildkielignenhglyladqyvkgiaksrks | |
| RF8135: RF8122 | mellilkanaittiltavtfcfasgqniteefyqstcsayskgylsalrtgwytsvitielsnikenk | 21 |
| like with K4 65N | ongtdakvklikqeldkyknavtelqllmgsgnvglggaiasgvayskvlhlegevnkiksallstnk | |
| and K47 ON | avvslsngvsvltfkvldlknyidkqllpilnkqscsisnpetviefqqknnrlleitrefsvnagvt | |
| mutations to | tpvstymltnsellslindmpitndqkklmsnnvqivrqqsysimsiikeevlayvvqlplygvidtp | |
| limit | cwklhtsplottntkngsnicltrtdrgwycdnagnvsffpqaetckvqsnrvfcdtmnsrtlpsevn | |
| proteolysis | lonvdifnpkydckimtsktdvsssvitslgaivscygktkotasnknrgiiktfsngodyvsnkgvd | |
| instability | tvsvgntlyyvnkqegnslyvngepiinfydplvfpsdefdasisqvnelingslafingsdellsgs | |
| | lgsesqvrqqfskdiekllneqvnkemqssnlymsmsswsythsldgaglflfdhaaeeyehakklii | |
| | flnennvpvqltsisapehcfegltqifqkayeheqhisesinnivdhaikskdhatfnflqwyvaeq | |
| | heeevfkdildkielignenhglyladqyvkgiaksrks | |
| RF8136: RF8122 | mellilkanaittiltavtfcfasgqniteefyqstcsayskgylsalrtgwytsvitielsnikenk | 22 |
| like with K4 65N, | ongtdakvklikqeldkyknavtelqllmgsgnvglggaiasgvayskvlhlegevnkiksallstnk | |
| K47 ON, K52 8N | avvslsngvsvltfkvldlknyidkqllpilnkqscsisnpetviefqqknnrlleitrefsvnagvt | |
| and K532N | tpvstymltnsellslindmpitndqkklmsnnvqivrqqsysimsiikeevlayvvqlplygvidtp | |
| mutations to | cwklhtsplottntkngsnicltrtdrgwycdnagnvsffpqaetckvqsnrvfcdtmnsrtlpsevn | |
| limit | lonvdifnpkydckimtsktdvsssvitslgaivscygktkotasnknrgiiktfsngodyvsnkgvd | |
| proteolysis | tvsvgntlyyvnkqegnslyvngepiinfydplvfpsdefdasisqvnelingslafingsdellsgs | |
| instability | gsesqvrqqfsndienllneqvnkemqssnlymsmsswsythsldgaglflfdhaaeeyehakkliif | |
| | lnennvpvqltsisapehcfegltqifqkayeheqhisesinnivdhaikskdhatfnflqwyvaeqh | |
| | eeevlfkdildkielignenhglyladqyvkgiaksrks | |
| RF8140: RF8122 | MELLILKANAITTILTAVTFCFASGQNITEEFYQSTCSAVSKGYLSALRTGWYTSVITIELSNIKENK | 23 |
| with R523Q in | CNGTDAKVKLIKQELDKYKNAVTELQLLMGSGNVGLGGAIASGVAVSKVLHLEGEVNKIKSALLSTNK | |
| the bull frog | AVVSLSNGVSVLTFKVLDLKNYIDKQLLPILNKQSCSISNPETVIEFQQKNNRLLEITREFSVNAGVT | |
| linker mutated | TPVSTYMLTNSELLSLINDMPITNDQKKLMSNNVQIVRQQSYSIMSIIKEEVLAYVVQLPLYGVIDTP | |
| to prevent | CWKLHTSPLCTTNTKNGSNICLTRTDRGWYCDNAGNVSFFPQAETCKVQSNRVFCDTMNSRTLPSEVN | |
| potential | LCNVDIFNPKYDCKIMTSKTDVSSSVITSLGAIVSCYGKTKCTASNKNRGIIKTFSNGCDYVSNKGVD | |
| proteolysis in | TVSVGNTLYYVNKQEGKSLYVKGEPIINFYDPLVFPSDEFDASISQVNELINQSLAFINQSDELLSGS | |
| CHO cells | GSESQVQQQFSKDIEKLLNEQVNKEMQSSNLYMSMSSWSYTHSLDGAGLFLFDHAAEEYEHAKKLIIF | |
| | LNENNVPVQLTSISAPEHCFEGLTQIFQKAYEHEQHISESINNIVDHAIKSKDHATFNFLQWYVAEQH | |
| | EEEVLFKDILDKIELIGNENHGLYLADQYVKGIAKSRKS | |
| Post-F, | MELLILKANAITTILTAVTFCFASGQNITEEFYQSTCSAVSKGYLSALRTGWYTSVITIELSNIKENK | 24 |
| benchmark | CNGTDAKVKLIKQELDKYKNAVTELQLLMQSTPATNNRARRELPRFMNYTLNNAKKTNVTLSKKRKRR | |
| control molecule | AIASGVAVSKVLHLEGEVNKIKSALLSTNKAVVSLSNGVSVLTSKVLDLKNYIDKQLLPIVNKQSCSI | |
| | SNIETVIEFQQKNNRLLEITREFSVNAGVTTPVSTYMLTNSELLSLINDMPITNDQKKLMSNNVQIVR | |
| | QQSYSIMSIIKEEVLAYVVQLPLYGVIDTPCWKLHTSPLCTTNTKEGSNICLTRTDRGWYCDNAGSVS | |
| | FFPQAETCKVQSNRVFCDTMNSLTLPSEVNLCNVDIFNPKYDCKIMTSKTDVSSSVITSLGAIVSCYG | |
| | KTKCTASNKNRGIIKTFSNGCDYVSNKGVDTVSVGNTLYYVNKQEGKSLYVKGEPIINFYDPLVFPSD | |
| | EFDASISQVNEKINQSLAFIRKSDELLGLEVLFQGPHHHHHHHHSAWSHPQFEK | |
| DS-CAV1, | mellilkanaittiltavtfcfasgoiniteefyqstcsayskgylsalrtgwytsvitielsniken | 25 |
| positive control | kongtdakvklikqeldkyknavtelqllmqstpatnnrarrelprfmnytlnnakktnvtlskkrkr | |
| molecule | rflgfllgvgsaiasgvavckvlhlegevnkiksallstnkavvslsngvsvltfkvldlknyidkql | |
| | lpilnkqscsisnietviefqqknnrlleitrefsvnagvttpvstymltnsellslindmpitndqk | |
| | klmsnnvqivrqqsysimciikeevlayvvqlplygvidtpcwklhtsplottntkegsnicltrtdr | |
| | gwycdnagsysffpqaetckvqsnrvfcdtmnsltlpsevnlonvdifnpkydckimtsktdvsssvi | |
| | tslgaivscygktkotasnknrgiiktfsngcdyvsnkgvdtvsvgntlyyvnkqegkslyvkgepii | |
| | nfydplvfpsdefdasisqvnekingslafirksdellsggssgssggsdiikllneqvnkemqssnl | |
| | ymsmsswcythsldgaglflfdhaaeeyehakkliiflnennvpvqltsisapehkfegltqifqkay | |
| | eheqhisesinnivdhaikskdhatfnflqwyvaegheeevlfkdildkielignenhglyladqyvk | |
| | giaksrksgs | |
| Wild-type, | MELLILKANAITTILTAVTFCFASGQNITEEFYQSTCSAVSKGYLSALRTGWYTSVITIELSNIKENK | 26 |
| Native RSV F (A2 | CNGTDAKVKLIKQELDKYKNAVTELQLLMQSTPPTNNRARRELPRFMNYTLNNAKKTNVTLSKKRKRR | |
| strain) | FLGFLLGVGSAIASGVAVSKVLHLEGEVNKIKSALLSTNKAVVSLSNGVSVLTSKVLDLKNYIDKQLL | |
| | PIVNKQSCSISNIETVIEFQQKNNRLLEITREFSVNAGVTTPVSTYMLTNSELLSLINDMPITNDQKK | |
| | LMSNNVQIVRQQSYSIMSIIKEEVLAYVVQLPLYGVIDTPCWKLHTSPLCTTNTKEGSNICLTRTDRG | |
| | WYCDNAGSVSFFPQAETCKVQSNRVFCDTMNSLTLPSEINLCNVDIFNPKYDCKIMTSKTDVSSSVIT | |
| | SLGAIVSCYGKTKCTASNKNRGIIKTFSNGCDYVSNKGMDTVSVGNTLYYVNKQEGKSLYVKGEPIIN | |
| | FYDPLVFPSDEFDASISQVNEKINQSLAFIRKSDELLHNVNAGKSTTNIMITTIIIVIIVILLSLIAV | |
| | GLLLYCKARSTPVTLSKDQLSGINNIAFSN | |
| RSV G A strain | MSKNKDQRTAKTLERTWDTLNHLLFISSCLYKLNLKSVAQITLSILAMIISTSLIIVAIIFIASANHK | 27 |
| Native | ITSTTTIIQDATNQIKNTTPTYLTQNPQLGISPSNPSDITSLITTILDSTTPGVKSTLQSTTVGTKNT | |
| UniProtKB/Swiss- | TTTQAQPNKPTTKQRQNKPPSKPNNDFHFEVFNFVPCSICSNNPTCWAICKRIPNKKPGKRTTTKPTK | |
| Prot: P27022.1 | KPTPKTTKKGPKPQTTKSKEAPTTKPTEEPTINTTKTNIITTLLTSNTTRNPELTSQMETFHSTSSEG | |
| | NPSPSQVSITSEYPSQPSSPPNTPR | |
| RSV G | NHKVTLTTAIIQDATSQIKNTTPTYLTQDPQLGISFSNLSEITSQTTTILASTTPGVKSNLQPTTVKT | 28 |
| ectodomain, | KNTTTTQTQPSKPTTKQRQNKPPNKPNNDFHFEVFNFVPCSICSNNPTCWAICKRIPNKKPGKKTTTK | |
| residues 66-297 | PTKKPTFKTTKKDHKPQTTKPKEVPTTKPTEEPTINTTKTNIITTLLTNNTTGNPKLTSQMETFHSTS | |
| | SEGNLSPSQVSTTSEHPSQPSSPPNTTRQ | |
| RSV G peptide A2 | Azido-PEG4-SGGSSGSSEEEGGSRQNKPNNDFHFEVFNFVPCSICSNNPTCWAICKRIPNKKEEE | 29 |
| for conjugation | | |
| with N-terminal | | |
| Azido linker and | | |
| flanking | | |
| glutamates (aa | | |
| 151-193) | | |
| CpG oligodeoxy- | T*G*A*C*T*G*T*G*A*A*C*G*T*T*C*G*A*G*A*T*G*A | 30 |
| nucleotide | | |
| (asterisks | | |
| indicate | | |
| phosphorothioate | | |
| linkages) | | |
| Replacement | GSGNVGL | 31 |
| sequence in | | |
| RF8117 | | |
| substituted for | | |
| positions 98-144 | | |
| of SEQ ID NO: 26 | | |
| (wild-type RSV | | |
| F) | | |
| RSV Gcc (central | RQNKPPNKPNNDFHFEVFNFVPCSICSNNPTCWAICKRIPNKK | 32 |
| conserved | | |
| region) A2 | | |
| strain res | | |
| 151-193 | | |
| RSV Gcc (central | RKNPPKKPKDDYHFEVFNFVPCSICGNNQLCKSICKTIPNKK | 33 |
| conserved | | |
| region) B1 | | |
| strain | | |
| Not Used | | 34-200 |
| bfpFerritin- | ESQVRQQFSKDIEKLLNEQVNKEMQSSNLYMCMSSWSYTHSLDGAGLFLFDHAAEEYEHAKKLIIFLN | 201 |
| N19Q/C31S/526C | ENNVPVQLTSISAPEHKFEGLTQIFQKAYEHEQHISESINNIVDHAIKSKDHATFNFLQWYVAEQHEE | |
| | EVLFKDILDKIELIGNENHGLYLADQYVKGIAKSRKS | |
| bfpFerritin- | ESQVRQQFSKDIEKLLNEQVNKEMQSSNLYMSMSSWSYTHSLDGAGLFLFDHAAEEYEHAKKLIIFLN | 202 |
| N19Q/C31S/572C | ENNVPVQLTCISAPEHKFEGLTQIFQKAYEHEQHISESINNIVDHAIKSKDHATFNFLQWYVAEQHEE | |
| | EVLFKDILDKIELIGNENHGLYLADQYVKGIAKSRKS | |
| bfpFerritin- | ESQVRQQFSKDIEKLLNEQVNKEMQSSNLYMSMSSWSYTHSLDGAGLFLFDHAAEEYEHAKKLIIFLN | 203 |
| N19Q/C31S/A75C | ENNVPVQLTSISCPEHKFEGLTQIFQKAYEHEQHISESINNIVDHAIKSKDHATFNFLQWYVAEQHEE | |
| | EVLFKDILDKIELIGNENHGLYLADQYVKGIAKSRKS | |
| bfpFerritin- | ESQVRQQFSKDIEKLLNEQVNKEMQSSNLYMSMSSWSYTHSLDGAGLFLFDHAAEEYEHAKKLIIFLN | 204 |
| N19Q/C31S/K79C | ENNVPVQLTSISAPEHCFEGLTQIFQKAYEHEQHISESINNIVDHAIKSKDHATFNFLQWYVAEQHEE | |
| | EVLFKDILDKIELIGNENHGLYLADQYVKGIAKSRKS | |
| bfpFerritin- | ESQVRQQFSKDIEKLLNEQVNKEMQSSNLYMSMSSWSYTHSLDGAGLFLFDHAAEEYEHAKKLIIFLN | 205 |
| N19Q/C31S/S100C | ENNVPVQLTSISAPEHKFEGLTQIFQKAYEHEQHISECINNIVDHAIKSKDHATFNFLQWYVAEQHEE | |
| | EVLFKDILDKIELIGNENHGLYLADQYVKGIAKSRKS | |
| bfpFerritin- | ESQVRQQFSKDIEKLLNEQVNKEMQSSNLYMSMSSWSYTHSLDGAGLFLFDHAAEEYEHAKKLIIFLN | 206 |
| N19Q/C31S/S111C | ENNVPVQLTSISAPEHKFEGLTQIFQKAYEHEQHISESINNIVDHAIKCKDHATFNFLQWYVAEQHEE | |
| | EVLFKDILDKIELIGNENHGLYLADQYVKGIAKSRKS | |
| bfpFerritin- | ESQVRQQFSKDIEKLLNCQVNKEMQSSNLYMSMSSWSYTHSLDGAGLFLFDHAAEEYEHAKKLIIFLN | 207 |
| N19Q/C31S/E12C | ENNVPVQLTSISAPEHKFEGLTQIFQKAYEHEQHISESINNIVDHAIKSKDHATFNFLQWYVAEQHEE | |
| | EVLFKDILDKIELIGNENHGLYLADQYVKGIAKSRKS | |
| Exemplary H. | ESQVRQQFSKDIEKLLNEQVNKEMNSSNLYMSMSSWCYTHSLDGAGLFLFDHAAEEYEHAKKLIIFLN | 208 |
| pylori Ferritin | ENNVPVQLTSISAPEHKFEGLTQIFQKAYEHEQHISESINNIVDHAIKSKDHATFNFLQWYVAEQHEE | |
| with bullfrog | EVLFKDILDKIELIGNENHGLYLADQYVKGIAKSRKS | |
| linker | | |
| Exemplary wild- | LSKDIIKLLNEQVNKEMNSSNLYMSMSSWCYTHSLDGAGLFLFDHAAEEYEHAKKLIIFLNENNVPVQ | 209 |
| type H. pylori | LTSISAPEHKFEGLTQIFQKAYEHEQHISESINNIVDHAIKSKDHATFNFLQWYVAEQHEEEVLFKDI | |
| ferritin | LDKIELIGNENHGLYLADQYVKGIAKSRKS | |
| (GenBank | | |
| Accession | | |
| AAD06160.1) | | |
| (without | | |
| bullfrog linker | | |
| or N-terminal | | |
| Met) | | |
| CpG (ISS-1018) | TGACTGTGAACGTTCGAGATGA | 210 |
| Trichoplusia ni | TQCNVNPVQIPKDWITMHRSCRNSMRQQIQMEVGASLQYLAMGAHFSKDVVNRPGFAQLFFDAASEE | 211 |
| heavy chain | REHAMKLIEYLLMRGELTNDVSSLLQVRPPTRSSWKGGVEALEHALSMESDVTKSIRNVIKACEDDS | |
| ferritin | EFNDYHLVDYLTGDFLEEQYKGQRDLAGKASTLKKLMDRHEALGEF | |
| | IFDKKLLGIDV | |
| Trichoplusia ni | ADTCYNDVALDCGITSNSLALPRCNAVYGEYGSHGNVATELQAYAKLHLERSYDYLLSAAYFNNYQT | 212 |
| light chain | NRAGFSKLFKKLSDEAWSKTIDIIKHVTKRGDFMNFDQHSTMKTERKNYTAENHELEALAKALDTQK | |
| ferritin | ELAERAFYIHREATRNSQHLHDPEIAQYLEEEFIEDHAEKIRTLAGHTSDLKKFITANNGHDLSLAL | |
| | YVFDEYLQKTV | |
| Pyrococcus | MLSERMLKALNDQLNRELYSAYLYFAMAAYFEDLGLEGFANWMKAQABEEIGHALRFYNY | 213 |
| furiosus | IYDRNGRVELDEIPKPPKEWESPLKAFEAAYEHEKFISKSIYELAALABEEKDYSTRAFL | |
| ferritin | EWFINEQVEEEASVKKILDKLKFAKDSPQILFMLDKELSARAPKLPGLLMQGGE | |
| human heavy | MTTASTSQVRQNYHQDSEAAINRQINLELYASYVYLSMSYYFDRDDVALKNFAKYFLHQSHEEREHA | 214 |
| chain ferritin | EKLMKLQNQRGGRIFLQDIKKPDCDDWESGLNAMECALHLEKNVQQSLLELHKLATDKNDPHLCDFI | |
| | ETHYLNEQVKAIKELGDHVTNLRKMGAPESGLAEYLFDKHTLGDSDQES | |
| human light | MDSKGSSQKGSRLLLLLVVSNLLLPQGVLASSQIRQNYSTDVEAAVNSLVNLYLQASYTYLSLGFYF | 215 |
| chain ferritin | DRDDVALEGVSHFFRELAEEKREGYERLLKMQNQRGGRALFQDIKKPAEDEWGKTPDAMKAAMALEK | |
| (signal peptide | KLNQALLDLHALGSARTDPHLCDFLETHFLDEEVKLIKKMGDHLTNLHRLGGPEAGLGEYLFERLTL | |
| is underlined) | KHD | |
| lumazine | MQIYEGKLTAEGLRFGIVASRFNHALVDRLVEGAIDCIVRHGGREEDITLVRVPGSWEIPVAAGELA | 216 |
| synthase from | RKEDIDAVIAIGVLIRGATPHFDYIASEVSKGLANLSLELRKPITFGVITADTLEQAIERAGTKHGN | |
| Aquifex aeolicus | KGWEAALSAIEMANLFKSLR | |
| bullfrog linker | ESQVRQQF | 217 |
| Cysteine- | CLVPRGSLEHHHHHH | 218 |
| Thrombin-His | | |
| Linker (cysteine | | |
| is double | | |
| underlined) | | |
| E. coli 6,7- | MNIIEANVATPDARVAITIARFNNFINDSLLEGAIDALKRIGQVKDENITVVWVPGAYELPLAAGAL | 219 |
| dimethyl-8- | AKTGKYDAVIALGTVIRGGTAHFEYVAGGASNGLAHVAQDSEIPVAFGVLTTESIEQAIERAGTKAG | |
| ribityllumazine | NKGAEAALTALEMINVLKAIKA | |
| synthase | | |
| 16 amino acid | GGGGSGGGGSGGGGSG | 220 |
| linker | | |
| 28 amino acid | GGSGSGSNSSASSGASSGGASGGSGGSG | 221 |
| linker | | |
| 46 amino acid | GGSGSASSGASASGSSNGSGSGSGSNSSASSGASSGGASGGSGGSG | 222 |
| linker | | |
| FR1 | GGSGSASAEAAAKEAAAKAGGSGGSG | 223 |
| FR2 | GGSGSASAEAAAKEAAAKEAAAKASGGSGGSG | 224 |
| 47 amino acid | SGGGSGSASSGASASGSSCSGSGSGSSSASSGASSGGASGGGSGGSG | 225 |
| linker | | |
| comprising a C | | |
| for conjugation | | |

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### 1. Design and Characterization of Modifications to RSV F Polypeptides

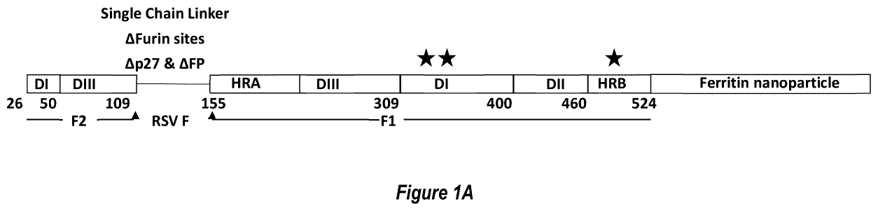

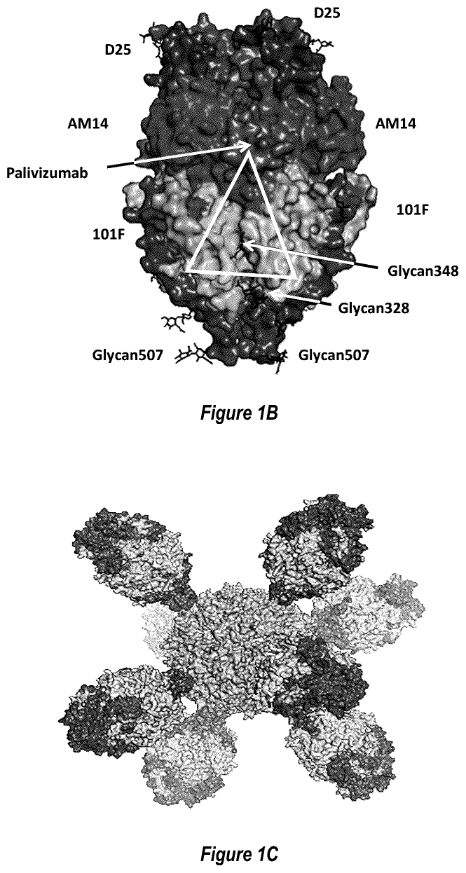

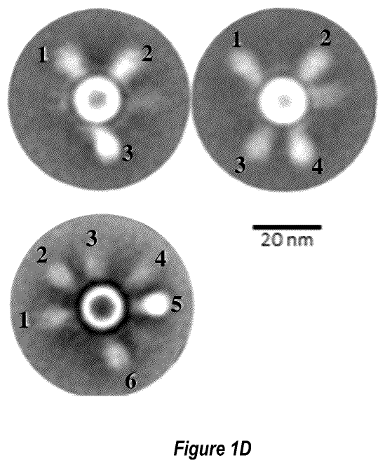

Like other paramyxovirus F proteins, RSV F is expressed as a precursor protein with an N-terminal signal peptide and a C-terminal transmembrane region that anchors the protein to the viral surface. RSV F undergoes intracellular cleavage by the protease furin to release a hydrophobic fusion peptide (“FP” in FIG. 1A), whose role is to attach to the target cell during infection. Adjacent to the fusion peptide is the heptad repeat region A (HRA) while the heptad repeat region B (HRB) is adjacent to transmembrane domain.

Crystal structures of RSV F ectodomain trimers in their pre-fusion and post-fusion conformations demonstrate how the HRA and HRB regions undergo significant rearrangement to drive the cellular fusion event (FIG. 1B) (see Swanson, K. A., et al., Proc Natl Acad Sci USA 108(23): p. 9619-24 (2011); McLellan, J. S., et al., Science 342(6158):592-598 (2013); McLellan, J. S., et al., J Virol 85(15):7788-96 (2011); and McLellan, J. S., et al., Science 342(6158): p. 592-8 (2013)). In the pre-fusion conformation, the heptad repeat A (HRA) region is associated with the globular head, and the tip of the fusion peptide is mostly buried in the center of the protein. The pre-fusion conformation contains a number of helices and involves certain contacts between protomers to form a pre-fusion trimer.

A series of amino acid substitutions were designed to be inter-protomer stabilizing. Exemplary substitutions include V207L; N228F; 1217V and E218F; 1221L and E222M; or Q224A and Q225L. All RSV F amino acid sequence numbering in the examples uses the numbering of SEQ ID NO: 26.

Amino acid substitutions were designed to be helix stabilizing. As such, these substitutions are predicted to stabilize the helical domain of RSV F. Exemplary substitutions include N216P or I217P.

Amino acid substitutions were designed to be intra-protomer stabilizing. Exemplary substitutions include V220I; or A74L and Q81L.

Amino acid substitutions were designed to be helix capping. Exemplary substitutions include N216P or I217P.

Amino acid substitutions were designed to decrease aggregation. Exemplary substitutions include V192E and L61Q.

Other amino acid substitutions were designed to be cavity-filling by introducing hydrophobic amino acids such as N228F

Amino acid substitutions E328N, S348N, and R507N were designed to add glycosylation sites by replacing non-asparagine residues with asparagine. It was hypothesized that addition of non-native glycans could be used to block epitopes that are exposed in the post-fusion RSV F (FIG. 1B) on the pre-fusion F protein surface.

RSV F constructs of interest were generated as single chain (scF) fusion proteins with a hybrid ferritin comprising an N-terminal bullfrog ferritin linker and H. pylori ferritin (pFerr) (FIG. 1A). The ferritin comprised a surface-exposed cysteine resulting from a K79C or S111C mutation (ferritin sequence numbering corresponds to SEQ ID NO: 208).

Generation of the various RSV Pre-F-NP and ferritin coding sequences was performed using standard cloning practices known in the field. Generally speaking, DNA for RSV F constructs with the described substitutions was synthesized and cloned into a mammalian expression vector by Genscript. RSV F DS-CAV1 and post-fusion F trimers were generated similarly to the protocols previously published (see McLellan, J. S., et al., Science 342(6158):592-598 (2013)). The DS-CAV1 construct retained the C terminal trimerization domain of RSV F and combined it with cavity-filling hydrophobic substitutions. The RSV F DS-CAV1 comprises a S155C-S290C disulfide multination (DS) and a S190F-V207L (CAV1).

Vectors encoding RSV F-ferritin nanoparticles, naked ferritin (i.e., not coupled to RSV F), and RSV F trimers were transfected into 293EXPI cells, and expression products were harvested from the conditioned media after 4 days. RSV F nanoparticles were purified by a series of anionic Q column purifications (GE Healthcare, Cat #17-1154-01) at pH 7.0 and 8.5 followed by Superose 6 SEC purification in PBS (GE Healthcare Cat #90-1000-42) using conventional chromatography methods. DS-CAV1 pre-fusion trimers and post-fusion trimers were stored at −80° C. and RSV F nanoparticles were stored at 4° C.

To determine the conformation of RSV F nanoparticles, electron microscopy was performed. RSV F nanoparticle preparations (30 g/mL in 25 mM Tris, 50 mM NaCl) were absorbed onto a 400-mesh carbon-coated grid (Electron Microscopy Sciences) and stained with 0.75% uranyl formate. A JEOL 1200EX microscope, operated at 80 kV, was used to analyze the samples. Micrographs were taken at 65,000× magnification and 2D class averages were prepared using conventional methods in the field by the EM company Nanoimaging Services, INC (San Diego, CA) (FIG. 1D).

Expression and secretion of polypeptides comprising these RSV F polypeptides and ferritin (SEQ ID NOs: 1-8 and 11-15) by transiently transfected 293 EXPI cells (Invitrogen) were evaluated by anti-RSV F Western blot. All anti-RSV F Western blots used the site 0-specific D25 antibody described in McLellan et al., Science 340(6136):1113-1117 (2013) and U.S. Pat. No. 8,562,996. As shown in FIG. 2, many constructs were successfully expressed and secreted.

The RF8085 polypeptide (SEQ ID NO: 1) represents a single chain mutant of the published DS-CAV1 RSV F (see McLellan, J. S., et al., Science 342(6158):592-598 (2013)) fused N-terminally to ferritin nanoparticle. This construct comprises a S155C-S290C double mutant (DS) of RSV F that retains antigenic site 0.

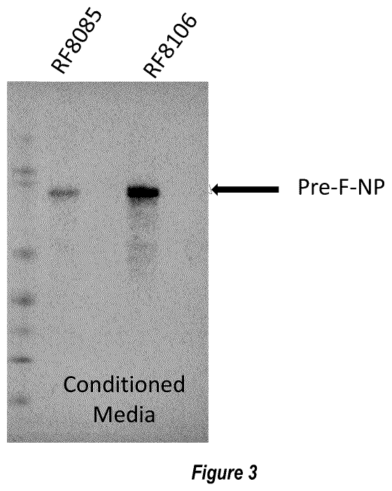

The RF8106 polypeptide (SEQ ID NO: 9) has an I217P substitution instead of the 2 cysteines substituted into DS-CAV1. As shown in FIG. 3, the RF8106 construct had significantly better expression in transiently transfected 293 EXPI cells as assessed from conditioned media after 4 days by anti-RSV F Western blot.

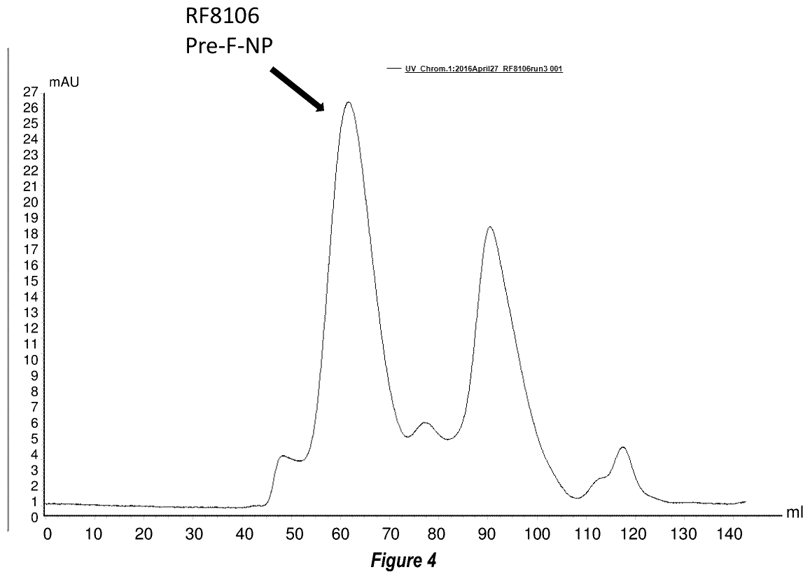

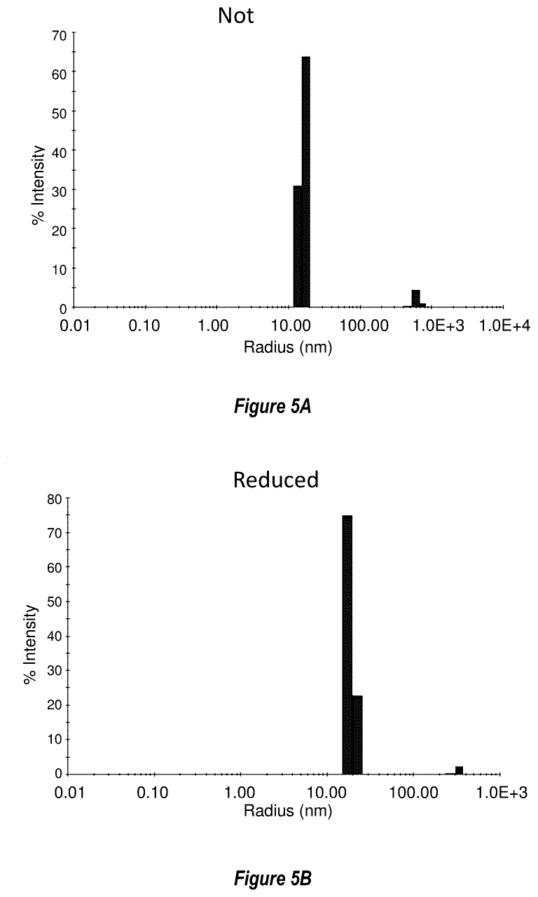

Size exclusion chromatography (SEC) of RF8106 showed elution of a main peak at a retention time consistent with an assembled ferritin particle fused to the RSV antigen consistent with a fusion protein nanoparticle (Pre-F-NP, FIG. 4). Dynamic light scattering (DLS) analysis of RF8106 was done in the reduced (FIG. 5B) and non-reduced states (FIG. 5A). Reduction was by treatment with 2 mM TCEP. RF8106 had a radius of approximately 15 nm, which is consistent with incorporation into a nanoparticle (24-mer) in both the reduced and nonreduced states. Stability of the fusion protein to reducing agents facilitates conjugation of adjuvants to the fusion proteins to form self-adjuvanting nanoparticles, as described below.

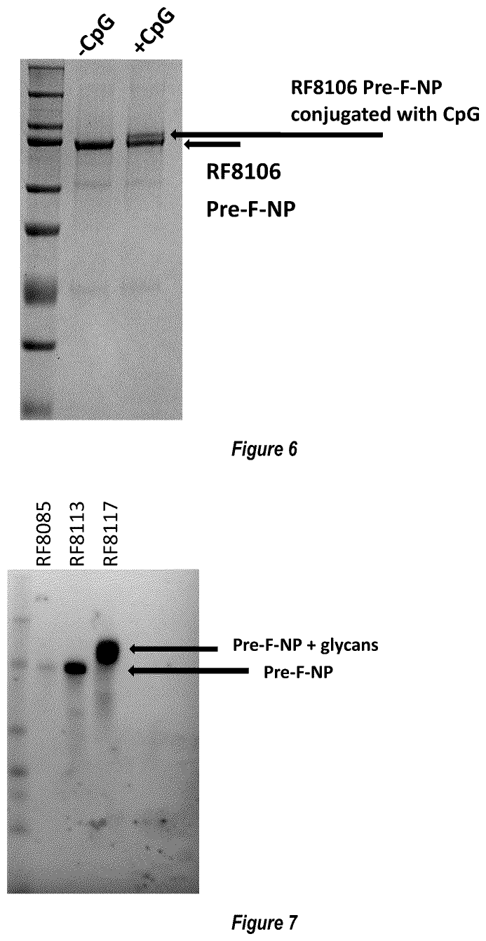

Next, conjugation of an adjuvant to the fusion protein of RSV F polypeptide and ferritin (Pre-F-NP) was assessed. It was found that the free surface cysteine on the ferritin can be used to attach an additional moiety to the scF-pFerr fusion protein. FIG. 6 shows successful conjugation of a CpG oligodeoxynucleotide (ODN) with the sequence T*G*A*C*T*G*T*G*A*A*C*G*T*T*C*G*A*G*A*T*G*A (SEQ ID NO: 30; asterisks indicate phosphorothioate linkages) to RF8106, as evidenced by an increase in the molecular weight as assessed by Coomassie-stained SDS-PAGE gel.

The effect of adding glycosylation sites using E328N, S348N, and R507N substitutions (RF8117, SEQ ID NO: 17) was assessed in 293EXPI cells transiently transfected with this construct as a fusion protein with ferritin (i.e., as Pre-F-NP constructs). RF8117 also contains an I217P substitution, as in RF8113. As shown in FIG. 7, increased expression was seen for RF8117 as compared with both the RF8085 control construct and the RF8113 construct (SEQ ID NO: 16, which comprises a proline substitution of I217P but not the E328N, S348N, and R507N substitutions). RF8113 is similar to RF8106 described previously except the engineered ferritin cysteine is on ferritin residue K79C rather than S111C. The RF8117 construct also showed an increase in the molecular weight of the RF8113 and RF8117, indicating the successful addition of glycans.

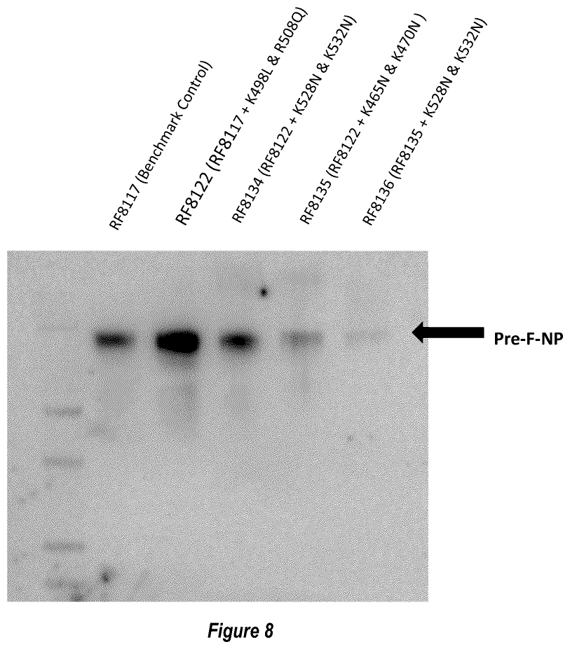

FIG. 8 summarizes modifications to RSV F nanoparticles that increased the proteolytic stability of the Pre-F-NP. The starting construct was RF8117 (above). When the earlier construct RF8085 was cloned into CHO vector as RF8090 and transfected into CHO cells, it was observed that some material was clipped between the F and ferritin moiety. It was suspected that arginine or lysine residues in the HRB region or the linker between the F and ferritin moiety were being cut by trypsin-like proteases. Mutations to lysine and arginine residues within the region were tested with respect to expression in 293 cells. FIG. 8 identifies mutations K498L and R508Q (in RF8122, SEQ ID NO: 18) as not affecting or increasing expression relative to RF8117. These mutations, with R523Q, were combined with the herein mentioned mutations of RF8117 to form construct RF8140 (SEQ ID NO: 23).

Greater improvements in expression (approximately 5-fold) were seen with the combination of single chain and proline (1217P) modifications in 293 cell expression (exemplary constructs with these substitutions include RF8106 (SEQ ID NO: 9) and RF8113 (SEQ ID NO: 16)) with further improvement in expression and solubility resulting from added glycosylation site modifications of RSV F (exemplary constructs RF8117 (SEQ ID NO: 17) and RF8140 (SEQ ID NO: 23). These constructs all have the fusion peptide and p27 peptide regions (amino acids 98-144 of SEQ ID NO: 26) replaced with the sequence GSGNVGL (SEQ ID NO: 31). However, when RF8090 was expressed in CHO manufacturing cell lines, additional RSV F bands in Western blots were observed, suggesting the construct was susceptible to proteolysis, perhaps trypsin-like cleavage at an arginine or lysine residue.

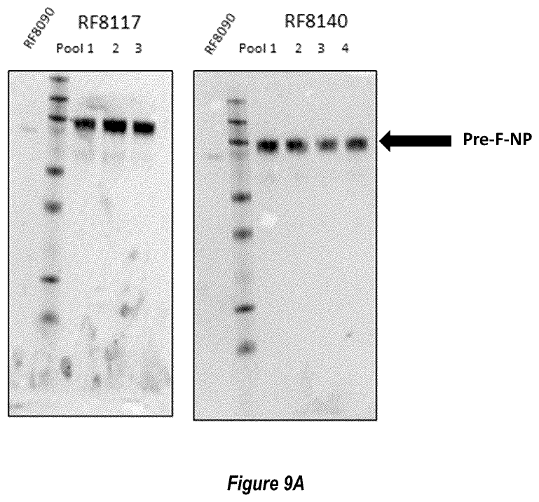

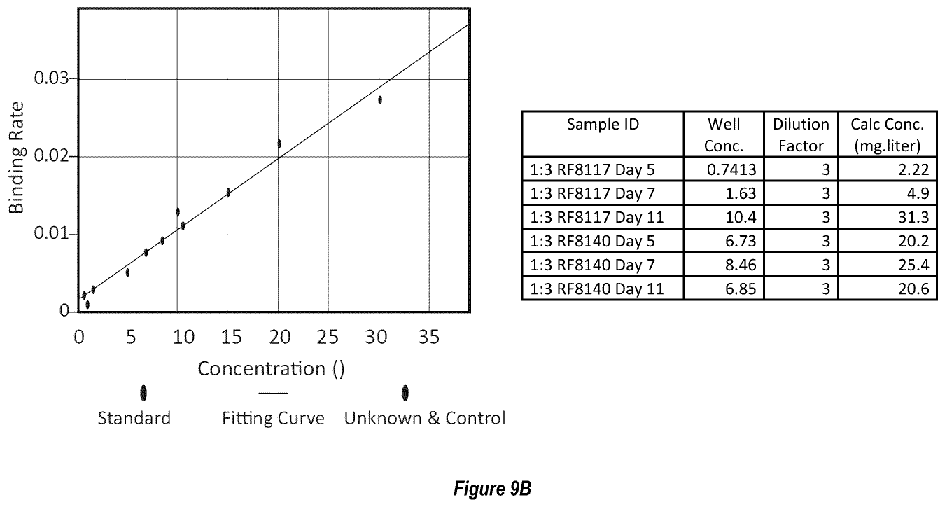

The potential role of protease susceptibility was also investigated. Substitution of K residues (knockout or KO) in the HRB region and in the linker between F moiety and ferritin moiety were made, as they were predicted to be possible sites of K-mediated cleavage initially observed in the CHO manufacturing cell line. As shown in FIGS. 9A and 9B, RF8117 and RF8140 both express to high levels relative to RF8090 in the CHO manufacturing cell line as measured by D25 Western blot or D25 and AM14 Octet analysis.

These data indicate that single chain constructs and amino acid modifications for helix capping, increasing glycosylation, and elimination of lysines or arginines susceptible to protease cleavage can improve expression of RSV F polypeptides, including RSV Pre-F-NP antigens.

### 2. Characterization of Fusion Proteins of RSV F and Ferritin Nanoparticles

Prior to animal studies, the concentration of DS-CAV1 and RSV F nanoparticles were analyzed by binding using Octet. The binding of the pre-fusion antigens to pre-fusion specific antibodies D25 and AM14 was also measured using a ForteBio Octet instrument. All assays were performed in PBS at 30° C. Antibodies were loaded onto Protein A (ProA) sensor tips (fortéBio #18-5013) for 400 seconds to allow capture to reach near saturation. Biosensor tips were then equilibrated for 90 seconds in PBS, followed by antigen association at known concentrations in PBS for 300 seconds, followed by dissociation of the antigen in PBS. Data analysis and curve fitting, assuming a 1:1 interaction, were carried out with Octet Data Analysis HT10.0 software using an external standard curve of binding of a purified Pre-F-NP at known concentration. An exemplary assay result to determine Pre-F-NP concentration in CHO conditioned media is shown in FIG. 9B.

### 3. In Vivo Characterization of Immune Response to RSV F Polypeptides

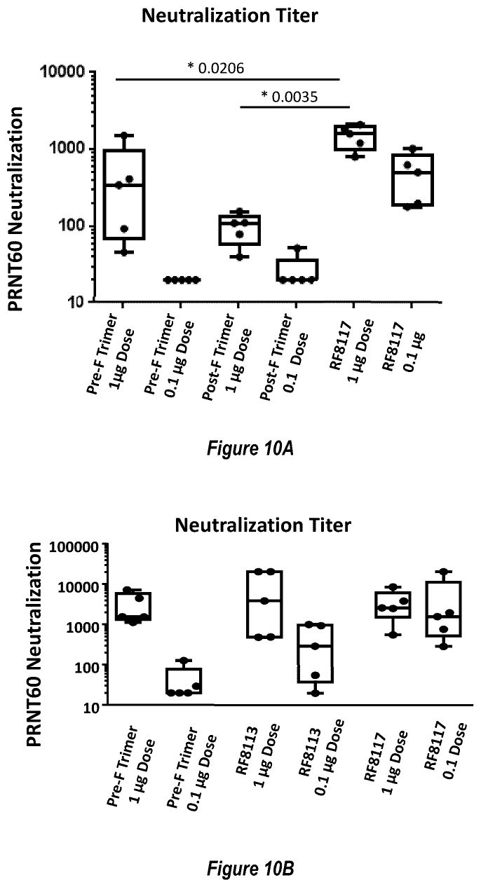

To assess the in vivo response to RSV antigens in mice, female BALBc mice were intramuscularly immunized with RSV antigens at specified doses at week 0, 3 and 6. Unless otherwise noted, RSV antigens (e.g., in the experiments of FIGS. 10A-B and 12A-B, among others) were adjuvanted with AF03 with a bedside mixing strategy. That is, 50 μl of the relevant protein solution were mixed with 50 μl of Sanofi adjuvant AF03 (a squalene-based emulsion; see Klucker et al., J Pharm Sci. 2012 December; 101(12):4490-500) just prior to injection of 50 μl into each hind leg. For unadjuvanted groups, antigens were mixed as above, but the AF03 was replaced with an equivalent volume of PBS. For antigens mixed with SPA09 or Alum, the above procedure was performed replacing the AF03 with an equivalent volume of SPA09 or Alum, respectively. No adverse effects from immunization were observed for any formulation. Blood was collected 1 day prior to first immunization and at least 2 weeks after each injection (i.e. weeks 2, 5 and 8). Unless otherwise specified, data shown was for 2 weeks post third injection (week 8, also denoted as 2wp3). Typically, sera were analyzed from pre-immunized animals (denoted as naïve), two weeks post second injection (post-2 or 2wp2) or two weeks post third injection (post-3^{rd} or 2wp3).

For the Vero cell neutralizing assay, serum was heat-inactivated for 30 minutes at 56° C. A four-fold serial dilution series of the inactivated serum was made in Dulbecco's Modified Eagle Medium (DMEM) supplemented with 2% Fetal Bovine Serum (FBS), 1% GlutaMAX, and 1% antibiotic-antimitotic. RSV viral stocks were combined 1:1 with the serum dilutions and incubated for 1.5 hours at 37° C. The virus-serum mixture was then added to 24 well plates containing confluent Vero cell monolayers at 100 μL per well and incubated for 1.5 hours at 37° C., 5% CO₂. The inoculum was then overlaid with 1 mL per well of 0.75% Methyl cellulose in DMEM supplemented with 2% FBS and 2% GlutaMAX and 2% antibiotic-antimitotic. Following 5 days of incubation at 37° C., 5% CO2, the overlay was removed and the monolayers were fixed with ice-cold methanol for 20 minutes.

The plates were then washed once in water and blocked with 5% non-fat dry milk in Phosphate Buffered Saline (PBS) for 30 minutes at room temperature with gentle agitation. The blocking solution was then replaced with 200 L per well of 2% dry milk in PBS containing a 1:2000 dilution of anti-RSV antibody conjugated to horse radish peroxidase (Abcam AB20686). Following 3 hours of incubation at room temperature, the plates were washed 2 times with water, developed with TrueBlue HRP substrate, washed twice more in water and air-dried.

The stained plaques were counted using a dissecting microscope. The neutralizing antibody titers were determined at the 60% reduction end-point of mock neutralized virus controls using the formula: 60% plaque reduction titer=(C/V×0.4−Low)/(High−Low)×(HSD−LSD)+LSD, where C/V=average of RSV plaques in mock neutralized virus control wells, Low and High are the average number of RSV plaques in the two dilutions which bracket the C/V×0.4 value for a serum sample, and the HSD and LSD are the Higher and Lower Serum Dilutions.

For the HAE neutralizing assay, serum was heat-inactivated for 30 minutes at 56° C. A fourfold serial dilution series of the inactivated serum was made in PneumaCult™-ALI Basal Medium (Stem Cell Technologies; 05002) supplemented with PneumaCult™-ALI 10× Supplement (Stem Cell Technologies; 05003) and 1% Antibiotic/Antimycotic (hence media). RSV viral stocks were combined 1:1 with the serum dilutions and incubated for 1.5 hours at 37° C. The virus-serum mixture was then added to 24 well plates containing fully differentiated HAE cells at 50 μL per well and incubated for 1.5 hours at 37° C., 5% CO₂. Following incubation, the inoculum was removed, the wells were washed twice with media to remove unbound virus and incubated a further 20 hours at 37° C., 5% CO₂. Infection events in cultures infected with RSV expressing the mKate (TagFP635) reporter were counted on a fluorescent microscope.

To detect infection with RSV not expressing the mKate reporter, the pseudostratified epithelia were washed extensively with media to remove mucus then fixed with 4% paraformaldehyde for 30 minutes at room temperature, permeabilized with 0.25% Triton X-100 for 30 minutes, and blocked with DMEM supplemented with 2% FBS for 1 hour at 37° C. The blocking solution was replaced with 100 μL per well of Mouse Anti-RSV monoclonal Ab mixture (Millipore; MAB 858-4) diluted 1:200 in DMEM supplemented with 2% FBS, and the plates were incubated at 37° C. for 2 hours. The plates were then washed 3 times with PBS supplemented with 0.05% Tween 20. 100 μL of Goat anti-mouse IgG (H+L) (Invitrogen; A11001) diluted 1:200 in DMEM supplemented with 2% FBS was added per well, and the plates were incubated overnight at 4° C. Next morning, the plates were washed 3 times with PBS supplemented with 0.05% Tween 20, the florescent signal was stabilized with ProLong Gold AntiFade with DAPI (Thermo Fisher Scientific; P36935) and counted on a fluorescent microscope. The neutralizing antibody titers were determined at the 60% reduction end-point as above.

For anti-F binding, either pre-fusion F (DS-CAV1) or post-fusion F were bound to anti-HIS antibody tips on the Octet. Unless specified, all anti-F binding refers to anti-pre-fusion F trimer (DS-CAV1) binding. His₆-tagged (SEQ ID NO: 230) RSV F trimer (DS-CAV1 or Post-fusion F were pre-loaded onto Anti-Penta-HIS (HIS1K) sensor tips (ForteBio #18-5122) for 400 seconds to allow capture to reach near saturation. Biosensor tips were then equilibrated for 90 seconds in Octet Wash Buffer, followed by diluted sera association for 300 seconds. Association curve final responses were measured using Octet Data Analysis HT10.0 software, and the response was multiplied by the dilution factor (100 or 300) to obtain the final reported response.

For anti-Gcc binding, a trimerized dimer of Gcc peptide with a C-terminal HIS tag was used on an Octet tip similar to above. His₆-tagged (SEQ ID NO: 230) Gcc (A2 strain) hexamer was pre-loaded onto Anti-Penta-HIS (HIS1K) sensor tips (ForteBio #18-5122) for 400 seconds to allow capture to reach near saturation. Biosensor tips were then equilibrated for 90 seconds in Octet Wash Buffer, followed by diluted sera association for 300 seconds. Association curve final responses were measured using Octet Data Analysis HT10.0 software, and the response was multiplied by the dilution factor (100 or 300) to obtain the final reported response.

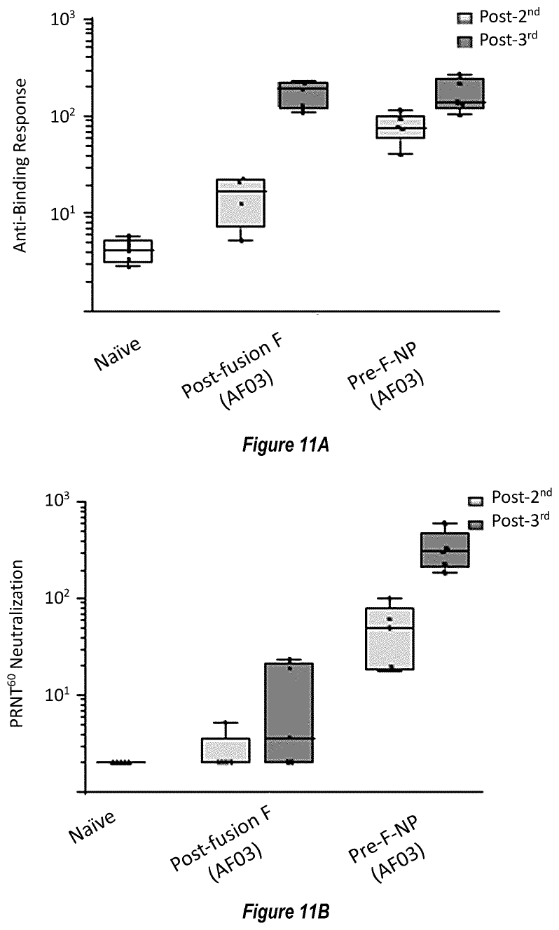

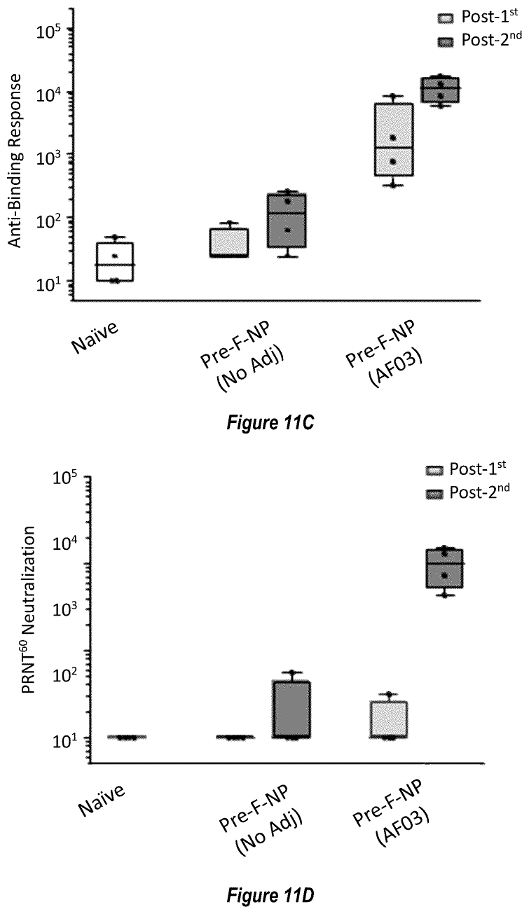

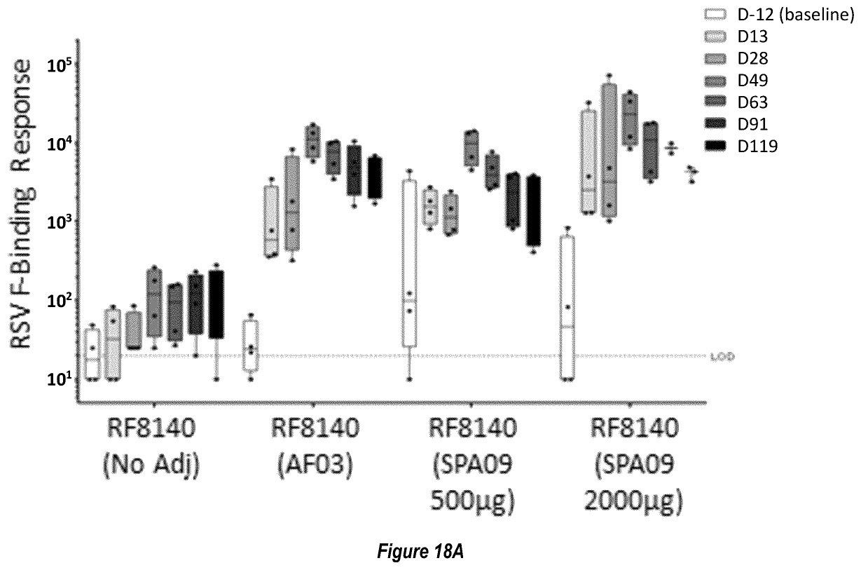

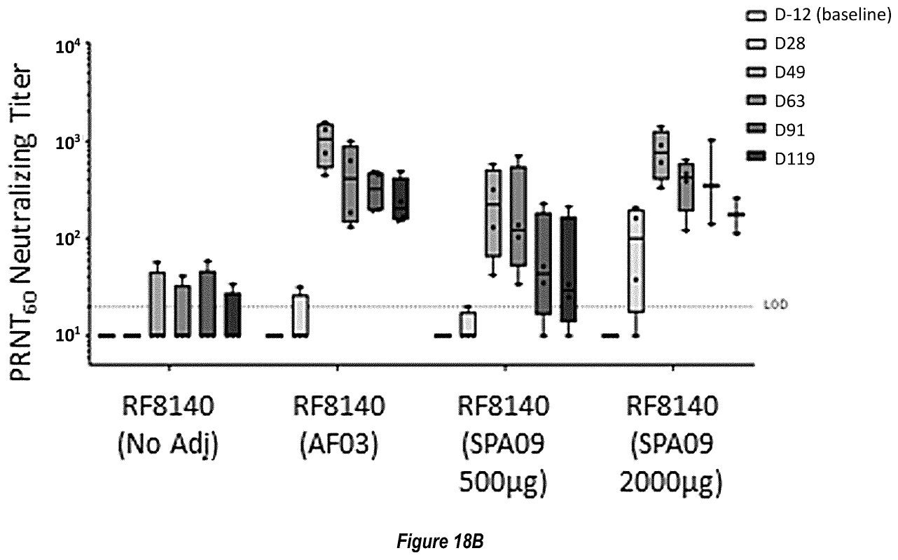

For non-human primate (NHP) studies, NHPs were pre-screened for RSV response (baselines were found to be below detection limits for all assays). NHPs were immunized with 50 μg of RF8140 with denoted adjuvant similar to the mouse protocol above but with larger volume of adjuvant (FIG. 11C-D and FIG. 18).

For non-human primate study, VERO neutralization assays were performed as described above. Pre-F-binding was assessed by ELISA assay below.

The NHP serum samples were serially diluted 2-fold (initial dilution 1:100) and incubated on blocked RSV soluble F (Sinobiological #11049-V08B) coated plates (1 μg/mL, 100 μL/well) for 1 h at 37° C. RSV F-specific IgGs were detected using horseradish peroxidase-conjugated anti-monkey IgG (BioRad AAI42P, 1:10,000 dilution) for 90 minutes at 37° C. Plates were developed using 3, 3′, 5, 5′-tetramethylbenzidine (TMB Tebu-Bio) and stopped with 1 N hydrochloric acid (Prolabo #30024290). The optical density (OD) was measured at 450 nm-650 nm with a microplate reader (SpectraMax). RSV sF-specific IgG titers were calculated using the SoftmaxPro software, for the OD value range of 0.2 to 3.0, from the titration curve (standard mouse hyper-immune serum put on each plate).

The IgG titers of this reference, expressed in arbitrary ELISA units (EU), corresponded to the log 10 of the reciprocal dilution giving an OD of 1.0. The threshold of antibody detection was 20 (1.3 log 10) EU. All final titers were expressed in log 10 for graphing. To each titer<1.3 log 10, an arbitrary titer of 1.0 log 10 was assigned.

To assess the cell mediated immunity in the NHP study, IFNγ/IL-2 FluoroSpot kit (FS-2122-10, Mabtech) was used following manufacturer's instructions. Briefly, membrane of the IPFL plates were pre-wet with 35% ethanol and the capture antibodies (anti-IFNγ and anti-IL-2) were coated overnight at 4° C.

Plates were then blocked for 2 hours at 37° C. with 200 μL/well of cell incubation medium containing 10% fetal calf serum (FCS). The medium was removed and the stimuli added in the wells: full-length F antigen (antigen-specific stimulation), anti-CD3 (positive control) or cell culture medium (unstimulated control). Macaque Peripheral Blood Mononuclear Cells (PBMCs) were thawed and numerated. 400,000 cells were added per well and incubated for 24 h at 37° C. in a humidified incubator with 5% CO2.

For detection the cells were removed and the detection antibodies (conjugated anti-IFNγ and anti-IL-2) were added and incubated 2h at room temperature. The fluorophore-conjugated reagents were then added and incubated 1 h at RT. Plates were empty, dried and stored in the dark at RT until analysis. Anti-CD3 mAb was used as positive control and responses of >500 Spot Forming Counts (SFC)/million PBMCs were found in all samples, verifying acceptable sample quality. Spots detected in the non-stimulated wells (cell culture medium) were subtracted to F-antigen stimulated cells.

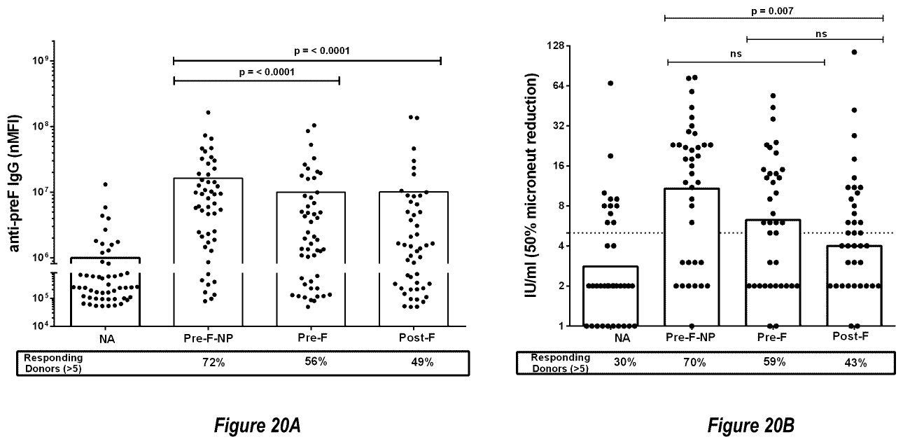

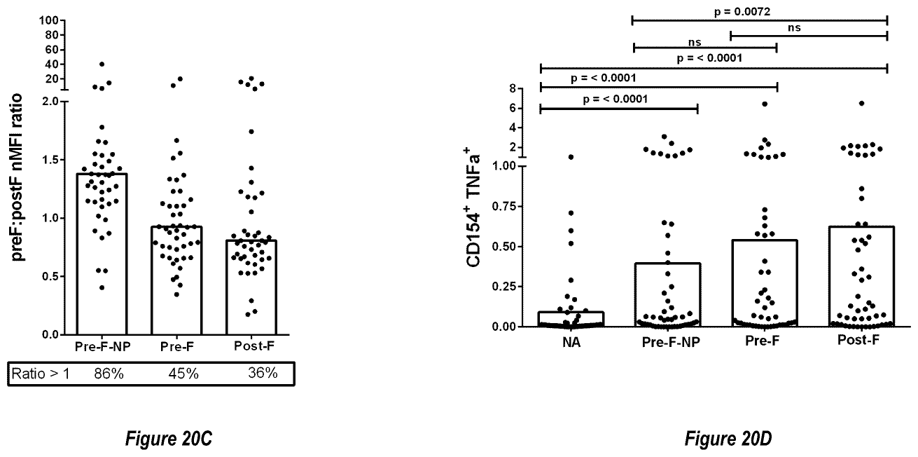

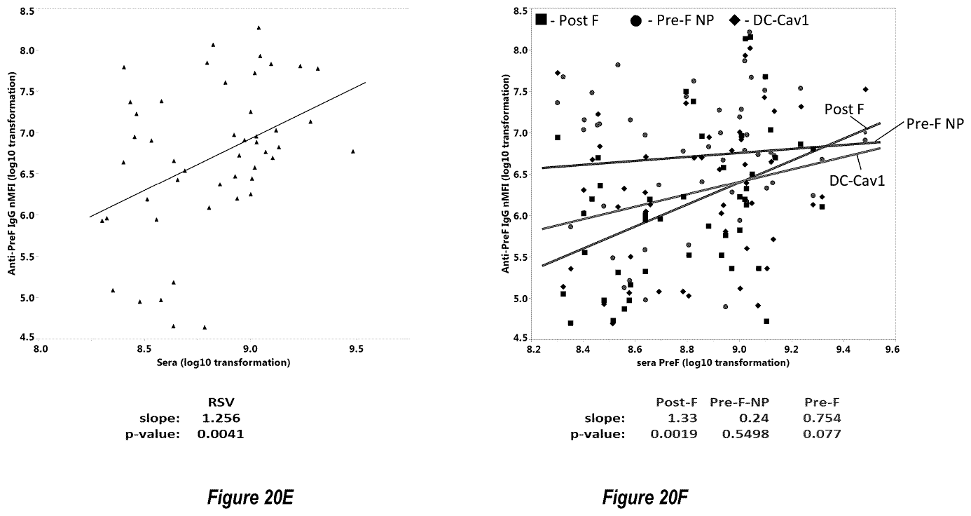

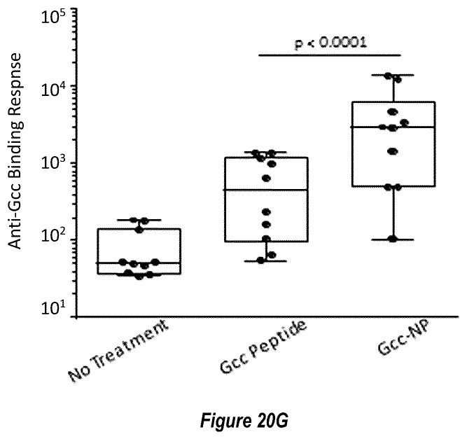

For the human cell (or B-cell) analysis, experiments were performed similar to referenced experiment Dauner, et al. Vaccine 2017 Oct. 4; 35(41):5487-5494 (FIG. 20). Cells were either not treated (treated with PBS) or treated with RSV F or RSV G polypeptides as denoted at 100 ng doses. F-binding and G-binding responses were performed using the luminex assay described in the literature with beads coated with pre-F-trimer (DS-CAV1) or G ectodomain, respectively.

RF8117 (SEQ ID NO: 17) comprises engineered glycosylation sites at E328N, S348N and R507N, which as mentioned above do not prevent D25 or AM14 binding. To demonstrate this pre-fusion nanoparticle elicits a similar immune response to other pre-fusion antigens (DS-CAV1) we immunized mice in groups of 5 with either pre-F trimer (DS-CAV1), post-fusion F or RF8117 at 1 μg or 0.1 μg doses, all adjuvanted with AF03, three times with three weeks between injections. Sera was tested for neutralizing titer two weeks after the third immunization using the VERO cell assay. RF8117 at the higher dose elicited a neutralizing titer similar to the pre-fusion control, and superior to the post-fusion control. At the lower dose, RF8117 elicited a higher neutralizing titer than both pre-fusion control and post-fusion control (FIG. 10A).

The RSV Pre-F-NP harbors glycosylation sites engineered to block epitopes shared between the pre-fusion and post-fusion confirmation. Whether these glycans were inhibiting the neutralizing response was evaluated. RF8117, with engineered glycans (SEQ ID NO: 17), was compared to RF8113 (similar to RF8117 but lacking the engineered glycans; SEQ ID NO: 16) and pre-fusion trimer control (DS-CAV1). Mice in groups of 5 were immunized with 1μ g or 0.1μ g doses, all adjuvanted with AF03, three times with three weeks between injections. Sera was tested for neutralizing titer two weeks after the third immunization using the VERO cell assay. There was no significant difference at either dose between the RF8113 and RF8117 constructs as judged by neutralizing titer (FIG. 10B).

To demonstrate that the herein mentioned lysine and arginine knockouts of RF8140 (SEQ ID NO: 23) do not upset the ability of the antigen to elicit a neutralizing response, we compared the immunogenicity of RF8140(SEQ ID NO: 25) to that of post-fusion F trimer (SEQ ID NO: 24) in mice (FIG. 11A&B). At low dose (0.1 μg) RF8140 (SEQ ID NO: 25) elicits a superior neutralizing titer to post-fusion trimer (SEQ ID NO: 24). To demonstrate RF8140 (SEQ ID NO: 23) elicits an immune response in NHPs, we immunized NHPs with RF8140 (SEQ ID NO: 25) with or without adjuvant (AF03). FIG. 11C shows the RSV F-binding response (ELISA titer) while FIG. 11D compares RSV neutralizing titers elicited by immunization with Pre-F-NP (RF8140, SEQ ID NO: 23). Both unadjuvanted and adjuvanted RF8140 (SEQ ID NO: 25) elicit an immune response in NHPs.

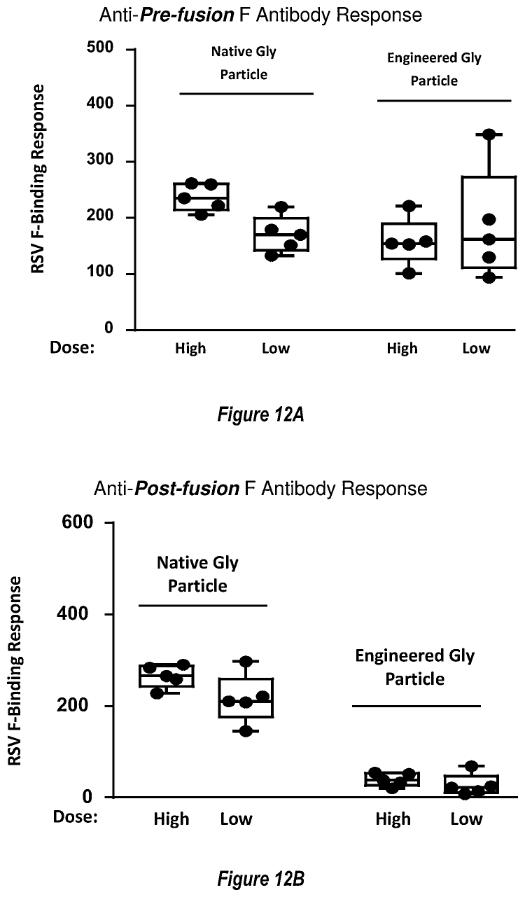

Having shown the engineered glycosylation sites of RF8117 (SEQ ID NO: 17) and RF8140 (SEQ ID NO: 23) do not prevent these antigens from eliciting a neutralizing response, we wanted to demonstrate they do block non- or poorly neutralizing epitopes shared between the pre-fusion and post-fusion conformation (FIG. 12). Antibody response to Pre-fusion F (DS-CAV1, SEQ ID NO: 25) elicited by immunization with Pre-F-NP without engineered glycosylation (RF8113, SEQ ID NO: 16) or Pre-F-NP with engineered glycosylation (Engineered Gly Particle, RF8117 SEQ ID NO: 17) at high (1 μg) and low (0.1 μg) dose as measured by Octet (FIG. 12A). Responses elicited by either Pre-F-NP were similar. Antibody response to post-fusion trimer elicited by immunization with Pre-F-NP without engineered glycosylation (RF8113, SEQ ID NO: 16) or Pre-F-NP with engineered glycosylation (RF8117, SEQ ID NO: 17) at high (1 μg) and low (0.1 μg) dose as measured by Octet (FIG. 12B). The post-fusion F-binding responses elicited by RF8117 (SEQ ID NO:17) were significantly lower than those elicited by RF8113 (SEQ ID NO: 16). Therefore, while both RF8113 and RF8117 elicit robust antibody responses to pre-fusion F, the post-fusion F antibody response elicited by RF8117 is greatly repressed. This is due to the engineered glycans mapping to the shared pre-fusion and post-fusion epitopes (FIG. 2B).

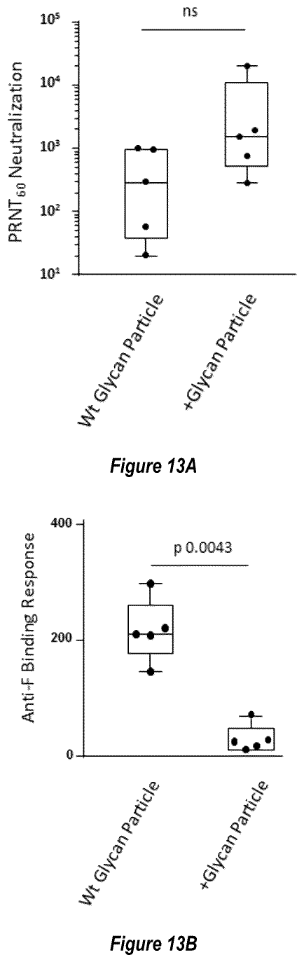

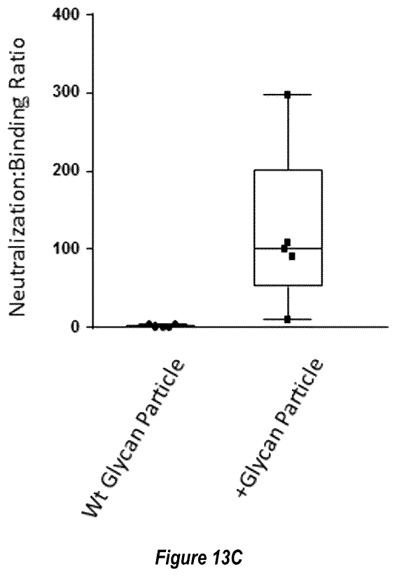

To further demonstrate that the engineered glycosylation sites block non-neutralizing epitopes but bias the neutralizing to non-neutralizing antibody titer, we analyzed the above data in a different way (FIG. 13). Comparison of RSV neutralizing titers as measured by VERO cell assay elicited by immunization with Pre-F NP with wild-type glycosylation sites (Wt Glycan Particle; RF8113, SEQ ID NO: 16) versus Pre-F NP with additional engineered glycosylation sites (+Glycan Particle; RF8117, SEQ ID NO: 17) in mouse studies were measured and showed no significant difference (FIG. 13A). Comparison of RSV Post-fusion F trimer-binding antibody responses elicited by immunization with Wt Glycan Particle (RF8113, SEQ ID NO: 16) versus+Glycan Particle (RF8117, SEQ ID NO: 17) in mouse studies showed a repressed post-fusion F-binding response for the Pre-F-NP with engineered glycans (FIG. 13B). To demonstrate that engineered glycans do not reduce the functional, neutralizing antibody response but decrease the non-neutralizing antibodies elicited to the shared pre-fusion/post-fusion epitopes, thus improving the neutralizing to total antibody ratio elicited by the engineered glycan constructs, the ratio of neutralizing titer to F-binding response was plotted (FIG. 13C). Therefore, the Pre-F-NPs with the engineered glycans elicit a superior neutralizing to binding antibody profile in mouse studies.

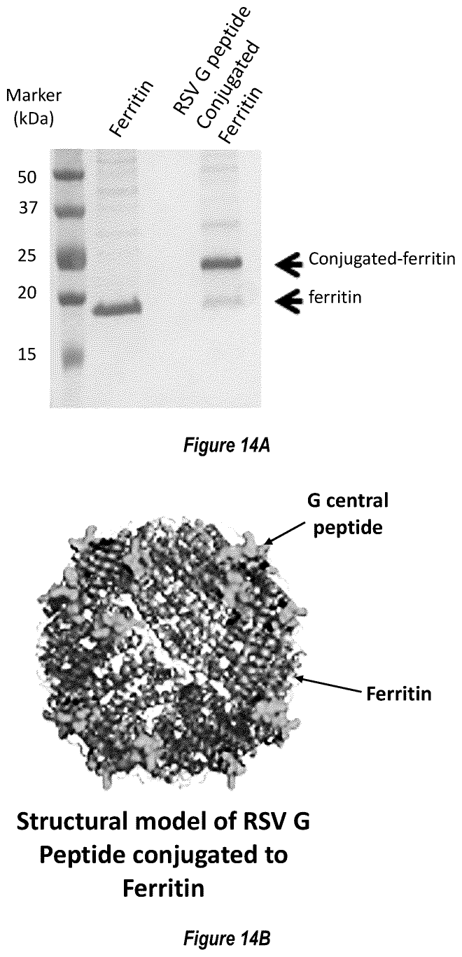

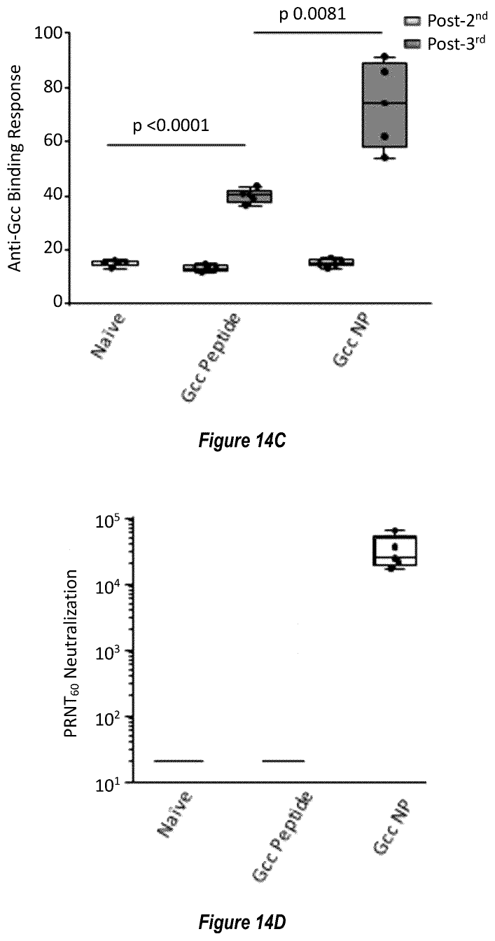

To demonstrate the ferritin nanoparticle can be used to improve the immunogenicity of the RSV G central domain antigen we developed a method of chemically conjugating the Gcc peptide (SEQ ID NO: 29) to the ferritin nanoparticle. Ferritin harboring the S111C mutation described herein can be conjugated with the Gcc peptide (SEQ ID NO: 29) synthesized with a maleimide group on a PEG4 linker attached to the N-terminus via a NHS group. Gcc peptide with an N-terminal maleimide was synthesized and HPLC purified by Peptides International (Louisville, KY, USA). When the maleimide-Gcc antigen is added to the ferritin S111C particle, the maleimide conjugates to the free cysteine and forms a Gcc-NP that can be observed by Coomassie-stained SDS-PAGE gel (FIG. 14A). While the conjugation is typically 50% to 90% efficient, a model of a Gcc peptide ferritin nanoparticle (100% conjugated) is shown in FIG. 14B.

To determine if the Gcc-NP elicits an immune response superior to the Gcc peptide (SEQ ID NO: 29), 5 mice per group were immunized with either Gcc peptide or Gcc-NP (1.3 μg dose mixed 1:1 with RIBI for each immunization). The Gcc-binding response (Octet) at two weeks post-second and two weeks post-third immunizations was compared to a representative group of naïve mice sera (FIG. 14C). The neutralizing response elicited by immunization with Gcc peptide (SEQ ID NO. 29) versus Gcc-NP in mouse studies post-third injection was also compared in HAE neutralizing assays (FIG. 14D). Gcc-NP elicits a superior immune response than Gcc peptide alone as judged by both Gcc-binding response and neutralizing response.

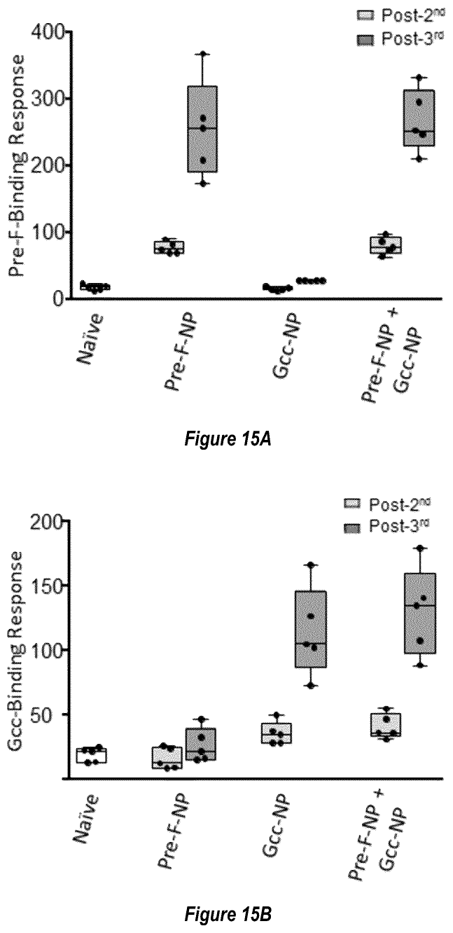

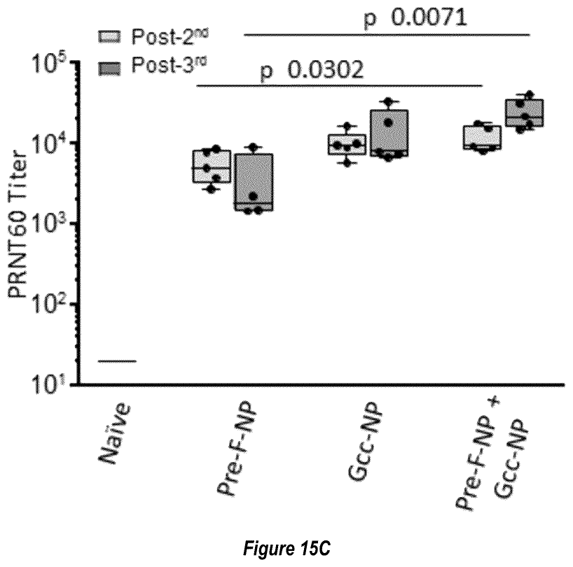

To demonstrate that co-administration of RSV Pre-F-NP (RF8140) and Gcc-NP does not interfere with either antigen's ability to elicit an immune response, mice were immunized with either Pre-F-NP alone (RF8140, SEQ ID NO: 23), Gcc-NP (ferritin conjugated with Gcc peptide SEQ ID NO: 29), or Pre-F-NP (RF8140, SEQ ID NO: 23) combined with Gcc-NP (FIG. 15A-C). All immunizations were adjuvanted with AF03. Mice immunized with RF8140 alone (Pre-F-NP) or RF8140 and Gcc-NP (Pre-F-NP+Gcc-NP) developed antibodies that bind pre-fusion F trimer (DS-CAV1, SEQ ID:25) while mice immunized with Gcc-NP did not. Mice immunized with Gcc-NP alone (Gcc-NP) or RF8140 and Gcc-NP developed antibodies that bind Gcc peptide, while mice immunized with just RF8140 did not. Animals immunized with either Pre-F-NP alone, Gcc-NP alone, or the co-administration of Pre-F-NP and Gcc-NP all developed a neutralizing response post-second and post-third immunization as measured by HAE neutralizing assay.

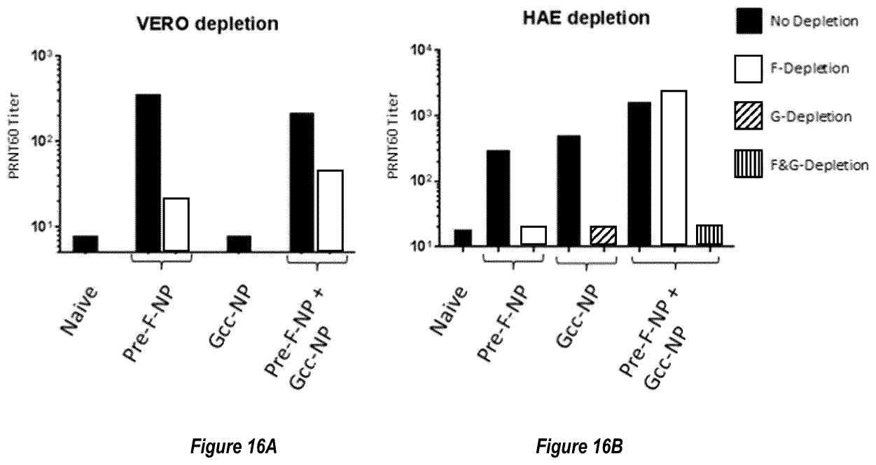

To determine if co-administration of RSV Pre-F-NP and Gcc-NP interfered with either antigen's ability to elicit neutralizing antibodies, neutralizing antibodies to both F and G were studied in a depletion assay (FIG. 16A-B). To demonstrate that the addition of Gcc-NP does not interfere with Pre-F-NP's ability to elicit a neutralizing response, the neutralizing titers were measured by the F-sensitive VERO cell assay for the groups mentioned above (FIG. 16A). Sera from naïve animals were also tested to judge the quality of the antigen depletions. In the VERO assay, sera from mice immunized with either RF8140 (SEQ ID NO: 23) alone or RF8140 mixed with Gcc-NP elicited similar neutralizing responses, while Gcc-NP did not appear to elicit neutralizing response in the F-antibody sensitive VERO assay. When antibodies that bind pre-fusion trimer (DS-CAV1, SEQ ID:25) were depleted from pooled sera from animals immunized with RF8140 (SEQ ID NO: 23) alone or immunized with RF8140 (SEQ ID:23) and Gcc-NP, a reduction in the measurable neutralizing titers was observed in the VERO assay. When the above groups were measured for neutralizing titer in the HAE cell assay, all immunization groups were observed to develop a neutralizing response in the F- and G-sensitive assay (FIG. 16B). Pooled sera from animals immunized with RF8140 (SEQ ID NO: 23) alone elicited a neutralizing response in the HAE assay that could be depleted out with pre-fusion F trimer (DS-CAV1, SEQ ID NO: 25). Pooled sera from animal immunized with Gec-NP alone elicited a neutralizing response in the HAE assay that could be depleted out with G ectodomain (SEQ ID NO: 28). Pooled sera from animals immunized with both Pre-F-NP (RF8140, SEQ ID NO: 23) and Gcc-NP elicited a neutralizing response in the HAE assay that was not fully depleted by DS-CAV1 (SEQ ID NO: 25) but was fully depleted by subsequent depletions with DS-CAV1 then G ectodomain (SEQ ID NO: 28). Together, these data suggest co-administration with the Pre-F-NP and Gcc-NP does not interfere with either antigen's ability to elicit neutralizing antibodies to pre-fusion F or G, respectively.

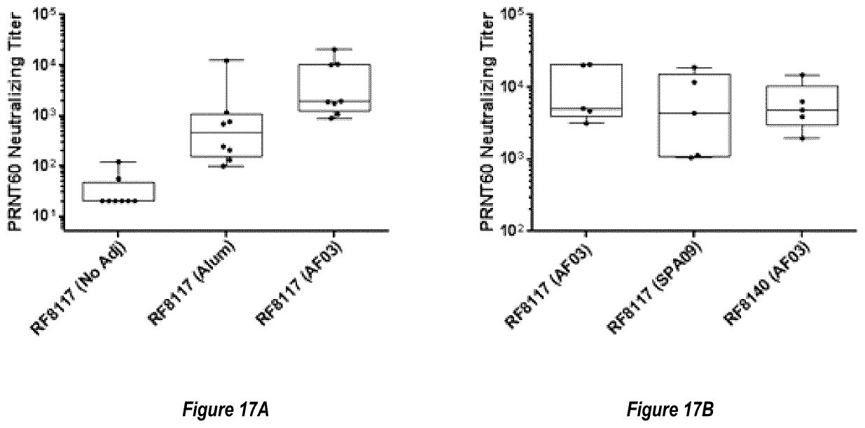

To demonstrate the effect of adjuvanting RF8117 (SEQ ID NO: 17) or RF8140 (SEQ ID NO: 23), mice were dosed with these constructs mixed with AF03, SPA09 or Alum. In FIG. 17A, mice were immunized with 10 μg antigen mixed with adjuvant, while in FIG. 17B, mice were immunized with 1 μg antigen mixed with adjuvant. In FIG. 17A, neutralizing titers measured by VERO cell assay at the two week post-third immunization timepoint. Sera from mice immunized with RF8117 (SEQ ID NO: 17) either unadjuvanted (No Adj), adjuvanted with Alum, or adjuvanted with AF03 are shown. In FIG. 17B, neutralizing titers were measured by VERO cell assay for sera from mice immunized with RF8117 (SEQ ID NO: 17) adjuvanted with AF03, RF8117 (SEQ ID NO: 17) adjuvanted with SPA09, or RF8140 adjuvanted with AF03. In all cases for either RF8117 (SEQ ID NO: 17) or RF8140 (SEQ ID NO: 23), in naïve mice adjuvanted groups elicit a higher neutralizing titer than non-adjuvanted groups. Mice immunized with RF8117 (SEQ ID NO: 17) or RF8140 (SEQ ID NO: 23) mixed with AF03 elicited a similar neutralizing response, suggesting the added lysine and arginine mutations of RF8140 (SEQ ID NO: 23) do not interfere with the Pre-F-NP's ability to elicit a neutralizing response.

To further explore the adjuvanting effect of AF03 and SPA09, non-human primates (NHPs) were immunized with RF8140 unadjuvanted, adjuvanted with AF03, or adjuvanted with two doses of SPA09 (FIG. 18A). NHPs were immunized with 50 μg of antigen mixed with indicated adjuvant at days 0 and 29 and immune response was measured by ELISA or VERO neutralizing response at indicated time points. Pre-fusion F trimer ELISA responses were measured in NHP sera after immunization with RF8140 either unadjuvanted (No Adj), adjuvanted with AF03, or adjuvanted with SPA09 (500 μg and 2000 μg doses of SPA09 were used). At all timepoints, adjuvanting with AF03 or SPA09 elicits a superior neutralizing response. Neutralizing titers of sera for the above NHP groups were also measured by VERO cell assay (FIG. 18B). In all cases immunization with RF8140 with adjuvant elicits a higher neutralizing titer than non-adjuvanted groups at all timepoints.

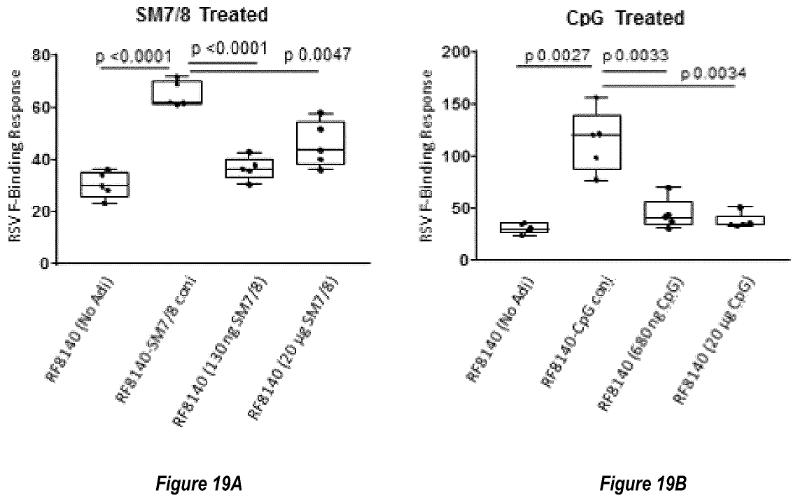

The effect of direct conjugation of RF8140 (SEQ ID NO: 23) to TLR7/8 agonist SM7/8 or TLR9 agonist CpG was tested. The antigen was conjugated with the small molecules SM7/8 or CpG and mice were dosed with 10 μg dose. RF8140 contains a mutation in its ferritin sequence replacing a surface exposed amino acid with a cysteine (K79C), which can be used for conjugation by click chemistry. For comparison, mice were dosed with RF8140 either unadjuvanted (No-adj), or adjuvanted by mixing with the small molecule at a high or low dose (not conjugated) as indicated in FIG. 19. Sera from animals post-second and post-third immunization was tested for Pre-fusion F trimer-binding.

In FIG. 19A, pre-fusion F trimer-binding response was measured in sera from either naïve mice, mice immunized with unadjuvanted RF8140 (SEQ ID NO: 23), mice immunized with RF8140 (SEQ ID NO: 23) conjugated with SM7/8 adjuvant, RF8140 (SEQ ID NO: 23) adjuvanted with 130 ng of SM7/8, or RF8140 (SEQ ID NO: 23) adjuvanted with 20 μg SM7/8. RF8140 (SEQ ID NO: 23) conjugated to SM7/8 elicits a higher pre-fusion F trimer-binding titer than unadjuvanted or SM7/8 adjuvanted groups.

In FIG. 19B, pre-fusion F trimer-binding response was also measured in sera from either naïve mice, mice immunized with unadjuvanted RF8140 (SEQ ID NO: 23), mice immunized with RF8140 (SEQ ID NO: 23) conjugated with CpG adjuvant, RF8140 (SEQ ID NO: 23) adjuvanted with 680 ng of CpG, or RF8140 (SEQ ID NO: 23) adjuvanted with 20 μg CpG. RF8140 (SEQ ID NO: 23) conjugated to SM7/8 elicits a higher pre-fusion F trimer-binding titer than unadjuvanted or SM7/8 adjuvanted groups.

To demonstrate the ability of the Pre-F-NP antigen and the Gcc-NP antigen to elicit a response in human cells, experiments were performed with the MIMIC platform (FIGS. 20A-D). The MIMIC platform is comprised solely of autologous human immune cells capable of quickly and reproducibly generating antigen-specific innate and adaptive responses upon challenge. Previous work has demonstrated the ability of the MIMIC system to recapitulate in vivo immune profiles against such diverse targets as HBV, tetanus toxoid, monoclonal antibodies, YF-VAX, and influenza B-cell responses. RSV Pre-fusion F trimer-binding antibody responses elicited by treatment with Pre-F-NP RF8140 (SEQ ID NO: 23) versus post-fusion F trimer (SEQ ID NO: 24) were compared in human B-cells, and a representative baseline response is shown for comparison (No Treatment) (FIG. 20A). Ratios of measured binding responses to pre-fusion F trimer (DS-CAV1, SEQ ID NO: 25) versus post-fusion F trimer (SEQ ID NO: 24) elicited by treatment with Pre-F-NP (RF8140, SEQ ID NO: 23) versus Post-fusion F (SEQ ID NO: 24) in human B-cells are shown in FIG. 20C. Antibodies from MIMIC elicited by treatment with different F antigens were measured using the VERO cell assay (FIG. 20B). Neutralizing titers elicited by treatment with Pre-F NP (RF8140, SEQ ID NO: 23) versus Post-fusion F trimer (SEQ ID NO: 24) in human B-cells were compared to a no treatment group showing RF8140 (SEQ ID NO: 23) elicited a superior neutralizing response in human cells.

The magnitude of Ab response to RSV infection or to F subunit vaccine candidates was determined based on the sero-status of the human subjects investigated in MIMIC studies, which was assessed by linear regression analysis. Donors with higher pre-existing circulating titers of anti-pre-F IgG produced significantly more anti-pre-F IgG after RSV treatment (FIG. 20E, p=0.0041) and after post-F priming (FIG. 20F, p=0.0019). Although the correlation did not reach statistical significance, pre-F also showed a relationship between the level of Ab induced and the level of pre-existing Ab. It is noteworthy that unlike other treatments, pre-F-NP induced comparably high level of anti-pre-F IgG from donors with low pre-existing anti-pre-F IgG as from donors with high pre-existing Ab (FIG. 20F). This indicates that pre-F-NP is capable of rescuing (or enhancing) Ab response even from donors with low pre-existing IgG level effectively.

To demonstrate Gcc-NP elicits a superior G antibody response than Gcc peptide (SEQ ID NO: 29) alone, human cells were treated with Gcc peptide alone (SEQ ID NO: 29) or Gcc peptide conjugated to nanoparticle (Gcc-NP) in human B-cells. Gcc-NP elicited a superior G-binding antibody response (FIG. 20G). Combined, these data suggest the Pre-F-NP and Gcc-NP will elicit immune responses in human immunization.

### 4. In Vivo Characterization of Immune Response to RSV Gcc-Ferritin Nanoparticles

RSV Gcc-NP was prepared as described above. To assess the in vivo response to RSV Gcc-NP in mice, female BALBc mice were intramuscularly immunized with RSV antigens at specified doses at week 0, 3 and 6 with either a high dose (5 μg) or low dose (0.5 μg) of antigen. Unless otherwise noted, RSV Gcc-NP was adjuvanted with AF03 with a bedside mixing strategy. That is, 50 μl of the protein solution was mixed with 50 μl of Sanofi adjuvant AF03 (a squalene-based emulsion; see Klucker et al., J Pharm Sci. 2012 December; 101(12):4490-500) just prior to injection of 50 μl into each hind leg. No adverse effects from immunization were observed. Blood was collected 1 day prior to first immunization and at least 2 weeks after each injection (i.e. weeks 2, 5 and 8). Unless otherwise specified, data shown was for 2 weeks post third injection (week 8, also denoted as 2wp3). Typically, sera were analyzed from pre-immunized animals (denoted as naïve), two weeks post second injection (post-2 or 2wp2) or two weeks post third injection (post-3^{rd} or 2wp3).

For the HAE neutralizing assay, serum was heat-inactivated for 30 minutes at 56° C. A fourfold serial dilution series of the inactivated serum was made in PneumaCult™-ALI Basal Medium (Stem Cell Technologies; 05002) supplemented with PneumaCult™-ALI lOX Supplement (Stem Cell Technologies; 05003) and 1% Antibiotic/Antimycotic (hence media). RSV viral stocks were combined 1:1 with the serum dilutions and incubated for 1.5 hours at 37° C. The virus-serum mixture was then added to 24 well plates containing fully differentiated HAE cells at 50 μL per well and incubated for 1 hour at 37° C., 5% CO₂. Following incubation, the inoculum was removed, the wells were washed twice with media to remove unbound virus and incubated a further 20 hours at 37° C., 5% CO₂. Infection events in cultures infected with RSV expressing the mKate (TagFP635) reporter were counted on a fluorescent microscope.

To detect infection with RSV not expressing the mKate reporter (RSV B strain neutralization), the pseudostratified epithelia were washed extensively with media to remove mucus then fixed with 4% paraformaldehyde for 30 minutes at room temperature, permeabilized with 0.25% Triton X-100 for 30 minutes, and blocked with DMEM supplemented with 2% FBS for 1 hour at 37° C. The blocking solution was replaced with 100 μL per well of Mouse Anti-RSV monoclonal Ab mixture (Millipore; MAB 858-4) diluted 1:200 in DMEM supplemented with 2% FBS, and the plates were incubated at 37° C. for 2 hours. The plates were then washed 3 times with PBS supplemented with 0.05% Tween 20. 100 μL of Goat anti-mouse IgG (H+L) (Invitrogen; A11001) diluted 1:200 in DMEM supplemented with 2% FBS was added per well, and the plates were incubated overnight at 4° C. Next morning, the plates were washed 3 times with PBS supplemented with 0.05% Tween 20, the florescent signal was stabilized with ProLong Gold AntiFade with DAPI (Thermo Fisher Scientific; P36935) and counted on a fluorescent microscope. The neutralizing antibody titers were determined at the 60% reduction end-point.

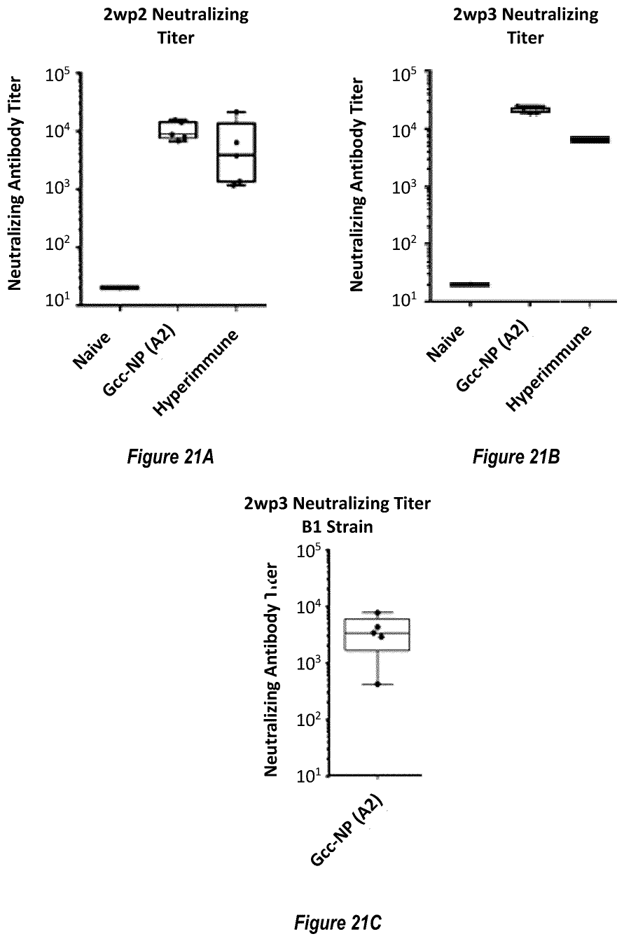

To demonstrate that higher multivalency improves elicitation of neutralizing response by RSV G antigens, mice were immunized with RSV F antigens. All immunizations were adjuvanted with AF03. Mice immunized with RSV Gcc-NP formulated with AF03 and neutralizing titers were measured at 2 weeks post second and 2 weeks post third injections (FIG. 21A-C). RSV Gcc-NP elicited a neutralizing response relative to naïve mouse sera. At both 2 weeks post second (FIG. 21A) and 2 weeks post third (FIG. 21B) immunization mice immunized with Gec-NP, containing Gcc from the A2 strain showed neutralizing responses for RSV A strain. At 2 weeks post third injection, Gcc-NP also elicited a neutralizing response for the RSV B1 strain (FIG. 21C).

For anti-Gcc binding, a trimerized dimer of Gcc peptide with a C-terminal HIS tag was used on an Octet tip. A His6-tagged Gcc (A2 strain) hexamer or His6-tagged Gcc (B1 strain) hexamer was pre-loaded onto Anti-Penta-HIS (HIS1K) sensor tips (ForteBio #18-5122) for 400 seconds to allow capture to reach near saturation. Biosensor tips were then equilibrated for 90 seconds in Octet Wash Buffer, followed by diluted sera association for 300 seconds. Association curve final responses were measured using Octet Data Analysis HT10.0 software, and the response was multiplied by the dilution factor (100 or 300) to obtain the final reported response.

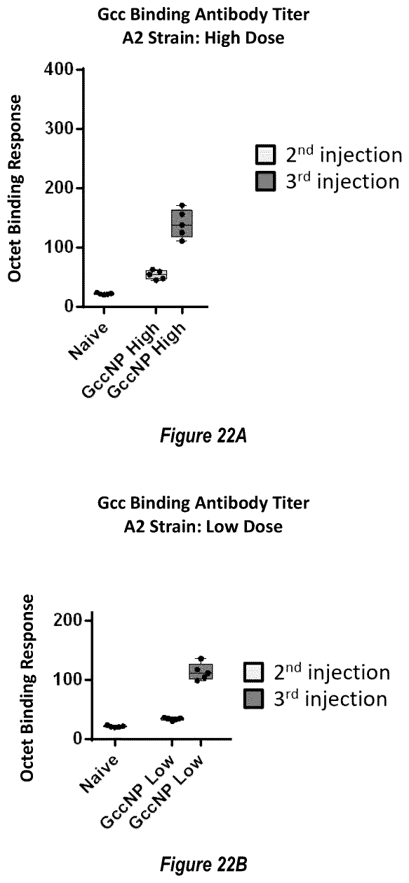

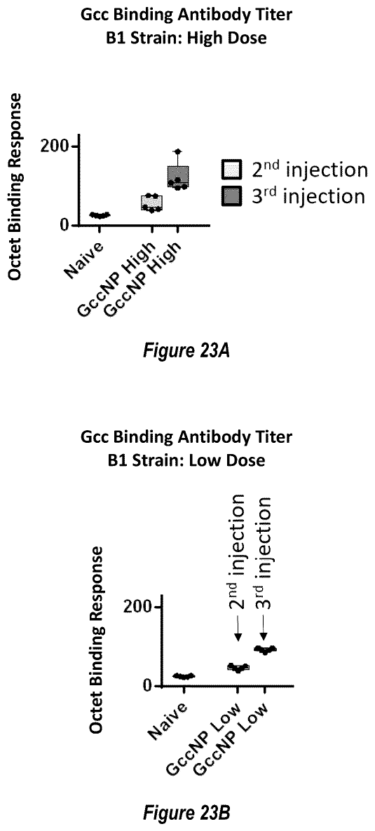

To determine if the RSV Gcc-NP elicits a Gcc-binding immune response, the sera from the immunizations described above were tested for their ability to bind Gcc A2 hexamer or Gcc B1 hexamer. The Gcc-binding response at high dose (FIG. 22A and FIG. 23A) and low dose (FIG. 22B and FIG. 23B) were tested at 2 weeks post-second and 2 weeks post-third immunizations. For both A2 strain (FIG. 22A-B) and B1 strain (FIG. 23A-B), RSV Gcc-NP elicited a binding response relative to naïve mice sera.

### 5. Response to Pre-F-NP and Gcc-NP in Human Cells

To demonstrate the ability of Pre-F-NP and Gcc-NP to elicit a response in human cells, experiments are performed with the MIMIC platform. The MIMIC platform is comprised solely of autologous human immune cells capable of quickly and reproducibly generating antigen-specific innate and adaptive responses upon challenge. Previous work has demonstrated the ability of the MIMIC system to recapitulate in vivo immune profiles against such diverse targets as HBV, tetanus toxoid, monoclonal antibodies, YF-VAX, and influenza B-cell responses. RSV Pre-fusion F trimer-binding antibody responses elicited by treatment with Pre-F-NP RF8140 (SEQ ID NO: 23) versus post-fusion F trimer (SEQ ID NO: 24) are compared in human B-cells, and are compared to a representative baseline response. Ratios of measured binding responses to pre-fusion F trimer (DS-CAV1, SEQ ID NO: 25) versus post-fusion F trimer (SEQ ID NO: 24) elicited by treatment with Pre-F-NP (RF8140, SEQ ID NO: 23) versus Post-fusion F (SEQ ID NO: 24) in human B-cells are determined. Antibodies from MIMIC elicited by treatment with different F antigens are measured using the VERO cell assay. Neutralizing titers elicited by treatment with Pre-F NP (RF8140, SEQ ID NO: 23) versus Post-fusion F trimer (SEQ ID NO: 24) in human B-cells are compared to a no treatment group, showing RF8140 (SEQ ID NO: 23) elicit a superior neutralizing response in human cells. To demonstrate Gcc-NP elicits a superior G antibody response than Gcc peptide (SEQ ID NO: 29) alone, human cells are treated with Gcc peptide alone (SEQ ID NO: 29) or Gcc peptide conjugated to nanoparticle (Gcc-NP) in human B-cells. Gcc-NP elicits a superior G-binding antibody response. Thus, Pre-F-NP and Gcc-NP will elicit immune responses in human immunization.

1.-39. (canceled)

40. An antigenic respiratory syncytial virus (RSV) polypeptide comprising an RSV F polypeptide, wherein an asparagine of the RSV F polypeptide is glycosylated, thereby blocking an epitope of the RSV F polypeptide that is shared between pre-fusion RSV F and post-fusion RSV F, wherein the RSV F polypeptide comprises a sequence having at least 90% identity to amino acids 1-478 of SEQ ID NO: 17, and wherein the asparagine corresponds to position 288, 308, or 467 of SEQ ID NO: 17.

41. The antigenic RSV polypeptide of claim 40, wherein the RSV F polypeptide is a pre-fusion RSV F.

42. The antigenic RSV polypeptide of claim 40, wherein the RSV F polypeptide is recognized by a pre-fusion RSV F-specific antibody selected from D25 or AM14.

43. The antigenic RSV polypeptide of claim 41, wherein the pre-fusion RSV F comprises an epitope not found on post-fusion RSV F.

44. The antigenic RSV polypeptide of claim 40, wherein the RSV F polypeptide is a post-fusion RSV F.

45. The antigenic RSV polypeptide of claim 40, wherein the asparagine corresponds to a non-asparagine residue in a wild-type RSV F sequence (SEQ ID NO: 26), wherein the non-asparagine residue corresponds to position 328, 348, or 507 of SEQ ID NO: 26.

46. The antigenic RSV polypeptide of claim 40, further comprising a ferritin protein.

47. An antigenic RSV polypeptide comprising an RSV F polypeptide and a ferritin protein, wherein the ferritin protein comprises a mutation replacing a surface exposed amino acid with a cysteine, and wherein the RSV F polypeptide comprises a sequence having at least 90% identity to amino acids 1-478 of SEQ ID NO: 17.

48. The antigenic RSV polypeptide of claim 47, wherein the ferritin comprises one or more of E11C, S25C, S71C, A74C, K78C, S99C, and S110C mutations corresponding to the H. pylori ferritin sequence of SEQ ID NO: 209 as determined by pair-wise or structural alignment, or one or more corresponding mutations in a non-H. pylori ferritin as determined by pairwise or structural alignment.

49. The antigenic RSV polypeptide of claim 46, comprising one or more immune-stimulatory moieties linked to the ferritin via a surface-exposed amino acid of the ferritin, wherein the surface-exposed amino acid is a cysteine resulting from a mutation.

50. The antigenic RSV polypeptide of claim 46, wherein the ferritin comprises a mutation replacing a surface-exposed asparagine with a non-asparagine amino acid, wherein the asparagine is at a position corresponding to position 18 of H. pylori ferritin sequence of SEQ ID NO: 209 as determined by pair-wise or structural alignment, or is at an analogous position in a non-H. pylori ferritin as determined by pairwise or structural alignment.

51. The antigenic RSV polypeptide of claim 46, wherein the ferritin comprises a mutation replacing an internal cysteine with a non-cysteine amino acid, wherein the internal cysteine is at position 30 of the H. pylori ferritin sequence of SEQ ID NO: 209, or is at a position that corresponds to position 30 of the H. pylori ferritin sequence of SEQ ID NO: 209 as determined by pair-wise or structural alignment.

52. The antigenic RSV polypeptide of claim 46, wherein the RSV F polypeptide comprises an epitope not found on post-fusion RSV F which is a site 0 epitope, wherein the site 0 epitope comprises amino acid residues 62-69 and 196-209 of SEQ ID NO: 26.

53. The antigenic RSV polypeptide of claim 40, wherein the RSV F polypeptide lacks a furin cleavage site, wherein a linker is present in place of the furin cleavage site.

54. A ferritin particle comprising the antigenic RSV polypeptide of claim 46.

55. A composition comprising the antigenic RSV polypeptide of claim 40 and an RSV G polypeptide.

56. A method of eliciting an immune response to RSV or protecting a subject against RSV infection comprising administering the antigenic RSV polypeptide of claim 40 to a human subject.

57. A nucleic acid encoding the antigenic RSV polypeptide of claim 40, wherein the nucleic acid is an mRNA or DNA.

58. An antigenic respiratory syncytial virus (RSV) polypeptide comprising a sequence having at least 90% identity to amino acids 1-478 of SEQ ID NO: 17.
